SEQUENCE LISTING (83 sequences)

SEQ ID NO: 1 - protein, 330 aa, Arthrospira platensis
MSDSPTPETSSEAIPNSGSKTRQLLGMKGAAPGETSIWKIRLQLMKPITWIPLIWGVVCGAASSGGYSWTLEDILKAAACMLLSGPLMAGYTQTLNDFYDRDLDAINEPYRPIPSGAISIPQVVSQILILLGAGIGLAYILDIWAGHEFPMVTVLCIGGAFVSYIYSAPPLKLKKNGWLGNYALGASYIALPWWAGHALFGELNPTIVVLTLFYSLAGLGIAIVNDFKSVEGDRQLGLQSLPVMFGVTTAAWICVLMIDIFQAGVALYLISIQQNLYATILLLLVIPQITFQDMYFLRNPLENDVKYQASAQPFLVLGMLVVGLALGHAV

SEQ ID NO: 2 - DNA, 993 bp, Arthrospira platensis
atgtctgattccccaacccccgaaacctcttcagaagccatccccaactccgggtcaaaaacccgccagttactagggatgaaaggagccgcccccggagaaacctccatctggaaaattcgcctgcaactgatgaaaccaatcacctggattcccctaatttggggtgtagtctgtggtgcggcttcctctggaggctatagctggacactagaagatatcctcaaagcagccgcctgtatgctgctgtcgggtcctttaatggcagggtacacccaaaccctcaacgacttctacgatcgcgacctagatgccattaacgagccctatcgccccattccctccggtgcaatttccatcccccaagtcgtatcccaaattctgattctcctaggtgcgggtattggtcttgcctatatcctggatatttgggcaggtcatgaattccccatggtcacagttctctgtattggcggcgctttcgtttcctatatctactctgcgcctcccctaaaactcaagaaaaacggctggttaggcaactatgccctcggagctagttatattgcccttccctggtgggcgggtcatgccctgtttggagaactcaaccccactattgtcgtactcaccctattctacagcttggcggggctaggtattgccattgtcaatgactttaagagtgtcgaaggcgatcgccaacttggcttacaatccctccccgttatgtttggcgtaaccaccgccgcctggatctgtgtattaatgatcgacatttttcaagccggagttgccctatacttgatcagcattcagcaaaacctgtatgccaccatccttttattactcgtgattccacaaattaccttccaagatatgtattttcttcgcaatcccctcgaaaatgatgttaaatatcaggcaagcgctcaacctttcctggtgttaggaatgttagtcgtgggtttagccctaggtcatgcagtttag

SEQ ID NO: 3 - protein, 162 aa, Arthrospira platensis
MKTPLTEAVSIADSQGRFLSSTEIQVAFGRFRQAKAGLEAAKALTSKADSLISGAAQAVYNKFPYTTQMQGPNYAADQRGKDKCARDIGYYLRMVTYCLIAGGTGPMDEYLIAGIDEINRTFELSPSWYIEALKYIKANHGLSGDAAVEANSYLDYAINALS

SEQ ID NO: 4 - DNA, 489 bp, Arthrospira platensis
ctagctcagggcgttgatcgcgtagtcgaggtaggagttagcttcaacagcagcgtcaccagacaaaccgtggttagctttgatgtatttcagggcttcaatgtaccagcttggagaaagctcgaaagtccggttgatttcatcaataccggcaatcaggtactcatccatggggccagttccaccagcaatcaggcaataagttaccatccgcaggtagtagcctatgtcacgagcacatttgtccttaccgcgttggtctgccgcgtagttaggtccctgcatttgggtggtgtaggggaacttgttgtacactgcttgggcagcaccactgatcagactatcagctttagaggtcaaagctttagcagcttccagaccagctttggcttgacgaaaacggccaaaagctacttggatttcggtgctgcttaggaaacgaccttgggaatcagcgatagaaactgcttcggttaggggggttttcat

SEQ ID NO: 5 - protein, 291 aa, Arthrospira platensis
MQDSESKTPGASPLVGADNAPLTGGEAEGGDAWTVEQAIAALNHQEPNRRYYAAWWLGRFRVDEPIAVDALIRALDDESDRTADGGYPLRRNAARALGKLGARRAVTPLVKCLDCPDFYVREAAAQALEGLGDSSCASALRGLLVGGVEDTEPIAGKPHLKQPYDAVIEAIGTLGATEALADIEPFINHPIARIQYAALRAMYQLTGDRVYGDRLIVALGGEDLQLRRSALMDLGAIGYLEAAEPIANTLAENSLKLIALQGILEHHLSQIGDKCLSEEAVKIMKLMDSLL

SEQ ID NO: 6 - DNA, 876 bp, Arthrospira platensis
atgcaggattctgaatcaaaaaccccaggtgcatcccccctagtgggcgcagataatgcgcctttaactgggggagaagctgaagggggtgatgcttggacagtagaacaggcgatcgccgccctaaaccatcaagaaccaaatcgtcgctattatgcggcttggtggttggggcgttttcgtgttgatgaaccgatcgcagttgatgcgttaattagggctttggatgatgagtccgatcgcacggccgacgggggctaccctttacggagaaatgcggctcgggctttgggtaaattgggcgcgcggcgcgcggtgactcccctagtcaaatgcttagattgcccagatttttatgtacgagaggcggcggctcaggctttggaaggtctgggagactctagttgtgcttcggctttgcgtgggttattggtgggtggagtagaagatactgaaccgatagcgggtaaaccccatttaaaacagccttatgatgcggtaattgaagccataggaaccctgggtgcaactgaggcgttagctgatattgaaccttttataaaccatccgatcgctagaatacaatatgcggctttgagggctatgtatcagcttacgggtgatcgggtttatggcgatcgcttaattgtggctttggggggggaagatttacaattgcgacgcagtgctttaatggatttaggggcgatcggataccttgaggcggcggaacctatcgccaataccctagctgaaaatagcctgaaactaattgctttacagggaattctggaacatcatctcagtcaaattggcgacaaatgcctatctgaagaggcagttaaaatcatgaagctgatggattctctattatga

SEQ ID NO: 7 - protein, 210 aa, Arthrospira platensis
MTRVEELILAVEQADSAGKLIGAVSQLAATGSVDAVPMLIRVLGYNNPGAAVAAVEGLIAIGKPAVVGLLERLDGYNYGARAWAVRALSGIGDPRGLDVLLEAAGSDFALSVRRAAARGLGAIHWEDMSSEVVSEAKERTLKTLLHTSEDTEWIVRYATVVGLEGLAASEVDKPNWFTQVAERLEGMVNNDETPAVSARASLARVNLGIS

SEQ ID NO: 8 - DNA, 633 bp, Arthrospira platensis
atgacaagggttgaggaactaattttagcggttgaacaggcggactctgcgggaaaactgataggggcggtgtcacaattggcggcgacgggttctgtggatgcagtaccaatgttaattcgtgttttgggttacaacaaccctggggcggcggtggcggcggttgagggattaatagccattggtaaacctgctgtggtggggctactagagaggttagatggttataactatggtgctagggcttgggcggtgcgggctttgtctggtattggtgatcctaggggtttggatgttttgctggaggcggcgggtagtgattttgctctgagtgtgcgtcgtgcggcggcgcggggtttgggagcaattcactgggaggatatgtcatcagaagtggtgtctgaggctaaggaaagaaccctaaaaactctgctgcatacttctgaagatacggaatggatcgttcgttatgcgacggttgtgggtttggaaggtttggcggcttctgaggtggataagcccaattggtttacacaggttgctgaaaggttggaggggatggttaataatgatgaaactccggcggtttctgctagggctagtctggcgagggtaaacttagggattagttaa

SEQ ID NO: 9 - protein, 770 aa, Prevotella sp.
MNLKTFTKTVLCFALFAVSAITAKAADHLAIVGEAVWGGWDLVKATAMVKSPNNPDVFMATVHLNAGKGFKFLTEREWGKLEYRSGASDVVLKSGIRYKLYASIGASEDGKFKVSESANYEIICDLARKTVEVKKVAYQAKEIRYAALWMIGDATAGDWDYNNGVLLSQDSGNPTCYTATVELKEGEFKFTTNKQWGYDHSVYIFRDVNDQNKIVFGGEDNKWRITEDGMYNVTVDVPTKTISIKQIDDPAGHKPQFGNDVILVGDATIAGWNLDNAIYLEHTGQAGRVFKTTTYLEAGKGFKFLSMLSYDDIDYRPANNTVLNPGVPGTFVPSLPSSTDTKFSVERSGNYDIVCNMNNRTVVVTLSENQVLVNYPALWLIGSATSAGWNPGKAVELKRSEADPAVYTARVQLKKGEFKILTSKNVGFDQPTYYRDSTNEHRIVFGVDGDEVAKKDCKWTLSENAEGTYDVTVDIEAMTIFCDKVNMDEPSVESTDKELILIGDATYSAWDLPKSIVMTPVGPTTFKAVTHLEAGKEFKFLTELAWKRYEYRAESLRKELQEGSMSMLVPYRYTNDKDDKDHDFKFVVKESGNYEIVCDLYIPALIIRKVRYQDTPVTYSSLWIVGSATPGGWTIERGIKMTQDENYPTKFTAKANLVPGELKFATNKFADFTQDFFFRGKDDYTAVLGGNDNKWNITEAGTYSVTIDVASKRVTITKPARNAPTGISTVDSSDEAPAEYFTLNGIKVTTPSSGIYIKRQGGRTTKVVMK

SEQ ID NO: 10 - protein, 193 aa, Prevotella sp.
MDTYQILDIIGCIVGLIYIYQEYKASIWLWMTGIIMPVIYMFVYYEAGLYADFGMQIYYTLAAIYGYLYWKLGKKKGTEDKEIPITHFPRRYIIPAIIVFFVLWIALYYILICFTNSTVPVLDSFGNALSFIGLWALAKKYLEQWWIWIVVDAELSALYIYKGIPFTAMLYALYTVIAVAGYFKWRRYIKQQK

SEQ ID NO: 11 - protein, 544 aa, Prevotella sp.
MRVRLYKNILLFLFLWVNTLACVSADTSRTVESQPIENGLIITESKGWLETIYAKWKPVAEADGYYVYVKGGQYADYSKVDSELIRVYNGYVRVDIPGLKAGTYSLKIVAVKGGKETQSSEVTGLKVLNYVREGFAHKNYSGVGAYNDDGTLKSGAVVIYVNKDNAKTVSAHLGKTTFIGLQAILNAYQKGNITTPLSVRILGLLRNGDTDTFGSSTEGIQIKGKQADSEMNITIEGIGEDASIYGFGFLVRNAKSVEFRNLGIMRAMDDGVSLDTNNSNIWIHHMDLFYGKASGGDHIKGDGSIDVKTDSKYVTIDNCHFWDTGKTSMCGMKKETGPNYITYHHNWFDHSDSRHARVRTMSVHLWNNYYDGCAKYGIGATMGCSVFSENNYFRATKNPILISKQGSDAKGTGKFSGEPGGMVKEYGSLFTEKGAESTYTPISYADNNSSFDFYHAISRNEKVPASVKTLNGGNIYNNFDTDAALMYSYTPDATALVPSQVTGFYGAGRLNHGSLQFKFNNAVEDTNSTPIPALEALIDAYSGK

SEQ ID NO: 12 - protein, 366 aa, Prevotella sp.
MKYNIAYCIEGFYNHGGMERILSVCANLLSDIYSITIIVANQRGREHAYNLAQNVNVVDLGVSCKNYKEEYKKSLTRYLQDHQFSVVISLAGLELFFLPQIKDGSKKVMWFHFAFDVSKMFLSERFHGWKLNLLYYIHTIRRIYFAKKFDTIVVLSKSDCDSWSRFCNNVKYIYNPITIDRKVISNLSEESVIAVGRLGWQKGFDFLIDSWVLVDDKHPDWHLDIFGEGPDRLELQHQIDRKGLHDKVRLCGVTKQIEEEYGKHSIYVMSSRAEGFPLALLEASSCGLPMISFNCHQGPNEIIQEGENGFLVDKVGDIYTLSDRICKLIEDNNLRNMMGKKALDSSFRFEGEVIKKDWISLLKQLI

SEQ ID NO: 13 - protein, 158 aa, Prevotella sp.
MKRLFFMFLFLGTITMNSLAQEEKPIKYETKNFSLPDKMPLYPGGDGALRAFLSLNLHYPEKAQAFGVEGRSLMKFCVSSDGSIKDISAVDCKITNYNRTEFNKLPLSKQESLKKECAKAFAKEAARVIRLMPKWEPAELNGKKMNVYYSLPFTFKLR

SEQ ID NO: 14 - protein, 415 aa, Prevotella sp.
MNYPLFIARKIYNGGDRTRKVSKPAIRIATIGVAIGLAVMIISVGVVLGFKHTIRNKVVGFGSDITVANFLTLQSSEQYPIQITDSLVKSLQITPGIKHVQRYDYTQGILKTDNDFLGVLLKGVGPDFDSTFIHENMVEGSLPHFHDNESQQKIVISKTIADKLNLKVGQRIFAYFINKQGVRTRKFTITGIYATNMKQFDSQICFTDIYTTNKLNGWEPDQYSGAELQVDNFSQLTPISMRVLNKVKNTVDHYGGTYSSENIIEQNPQIFSWLDLMDMNVWIILALMISVAGVTMISGLLIIILERTQMIGILKALGSRNRQIRHIFLWFATFIIGKGLLWGNIIGLGCILFQSWTGLVKLDPQTYYVNTVPVEINIPLIIALNMVTMLVCLVILIAPSYLISHIHPAKSMHYE

SEQ ID NO: 15 - protein, 736 aa, Prevotella sp.
MEDKFIYTDKERKLSYQILDELKDTLDKSFLENDLPMLQVQLKDSVAKNTIHRNVFGLNPILCSLQTAAIAVKDIGLKRDSVIAILLHQSVQDGYITLEDIDNRFGKSVAKIIHGLIRIQTLYQKNPIIESENFRNLLLSFAEDMRVILIMIADRVNLMRQIRDAEDKEAQHKVAEEASYLYAPLAHKLGLYQLKRELEDLSLKYLEHDAYYLIKDKLNATKASRDAYINQFIAPVRERLTAGGLRFHIKGRTKSIHSIWQKMKKQKCGFEGIYDLFAIRIILDAPLEKEKIQCWQAYSIVTDMYQPNPKRLRDWLSVPKSNGYECLHITVLGPEKKWVEVQIRTERMDEIAEHGLAAHWRYKGIKEEGGLDDWLASIRAALEAGDNLEVMDQFKSDLYEKEIYVFTPKGDLLKFPKGATILDFAYHIHSKVGNQCVGGKINAKNVSLRTELHSGDTVEILTSATQKPKAEWLKIVKSSRAKAKIRLALKETQIKDGLYAKELLERRFKNKKIEIEESTMGHLLRKLGFKEVSEFYKQVADEKLDPNYIIEEYQKVYNHDHNLNQPKETESAENFEFENPTNEFLKKNDDVLVIDKNLKGLDFSLAKCCHPIYGDPVFGFVTVNGGIKIHRTDCPNAPEMRKRFGYRIVKARWSGKGSSQYAITLRVIGNDDIGIVSNITNVISKDEKIVMRSINIDSHDGLFSGNLVVLLDDNSKLNMLIKKLRTVKGVKQVTRI

SEQ ID NO: 16 - protein, 336 aa, Prevotella sp.
MKRRIFLFVALSVSIVILFGLNLIIGSVHIPLSDILTILSGSFTGKESWRFIIWDSRLPQALTAMLCGSSLAVCGLMLQTAFRNPLAGPDVFGISSGASLGVALVMLLLGGTVETSMFTASGFLAILIVAFAGAILVTAFILFLSSVVRNSVLLLIVGIMVGYVASSAVTLLNFFSSEDGVKGYIVWGMGNFGGVSMSHIPLFAFLCLAGIIASFLLVKPLNILLLGPQYAESLGISIRRIRNILLVVVGILTAVTTAFCGPISFIGLAAPHVARLLFRTENHQKLLPGTLLVGTVVALLCNLICFLPRESGMIPLNAVTPLIGAPIIIYVIMKRH

SEQ ID NO: 17 - protein, 524 aa, Prevotella sp.
MKLENKEFGFDSFATEMARLKNEKHFDYLVTVVGEDFGTEEGLGCIYILENTSTHERCSVKQLAKKVGEEFVIPSVIKLWADADLLEREVYDFYGIKFLGHPDMRRLFLRNDFKGYPLRKDYDMDPAKNMYTTEDDVELDTTTEWNLDKNGELVGTQHALFTDDNFVVNIGPQHPSTHGVLRLQTVLDGETVTNIYPHLGYIHRGIEKLCEQFTYPQTLALTDRMNYLSAMMNRHALVGVIEEGMGIELSERILYIRTIMDELQRIDNHLLYTACCAQDLGALTAFLYGMRDREHVLNVMEETTGGRLIQNYYRIGGLQADIDPNFVSNVKELCKYLRPMIQEYVDVFGDNVITHQRFEGVGVMDEKDCISYGVTGPAGRASGWKNDVRKYHPYAMYDKVNFEEITLTNGDSMDRYFCHIKEIYQSLNIIEQLIDNIPEGEFYIKQKPIIKVPEGQWYFSVEGASGEFGAYLDSRGDKTAYRLKFRPMGLTLVGAMDKMLRGQKIADLVTTGAALDFVIPDIDR

SEQ ID NO: 18 - protein, 142 aa, Prevotella sp.
MRTSTQSKDMGKKQEYKLRNEEFLHNISKKDSIKTLPHGIFYEIIKEGSGEGTVQPRSIVICNYRGSLISGQVFDDSWQKPTPEAFRLNELITGLQIALCAMHKGDSWRIYIPYQEGYGSKRNADIPAFSTLIFDIELINIA

SEQ ID NO: 19 - protein, 196 aa, Prevotella sp.
MADNKIAKESVKREVIAGERLYTLLVYSENVAGVLNQIAAVFTRRQVNIESLNVSASSIEGIHKYTITAWSDAATIEKITKQVEKKIDVIKADYYEDSDLFIHEVGLYKIATPILLENAEVSRAIRKRNARMMEVNPTYSTVLLAGMTDEVTALYHDLKNFDCLLQYSRSGRVAVTRGFSEPVSDFLKSEEESSVL

SEQ ID NO: 20 - protein, 390 aa, Prevotella sp.
MKKKVKIGLLPRVIIAILLGIFFGYFMPTPLARVFLTFNGIFSQFLGFMIPLIIIGLVTPAIADIGKGAGKLLLVTVIIAYVDTVVAGGLAYGTGLCLFPSMIASTGGAMPHIDKATELAPYFSINIPAMADVMSGLVFSFMLGLGIAYGGLTATKNIFNEFKYVIEKVIAKAIIPLLPLYIFGVFLNMAHNGQAQQILLVFSQIIIVILVLHVFILVYQFCIAGAIIRRNPFRLLWNMMPAYLTALGTSSSAATIPVTLEQTMKNGVGKEIAGFVVPLCATIHLSGSAMKITACALTICLLVGLPHDPALFIYFILMLSIIMVAAPGVPGGAIMAALAPLASILGFNSEAQALMIALYIAMDSFGTACNVTGDGAIALVVNKMFGKKER

SEQ ID NO: 21 - protein, 162 aa, Prevotella sp.
MKKLLLLVCAAVMSLSASAQAGDKALGAQLVFGSETNSLGFGVKGQYYFTDHIRGEGSFDYFLKNKGISMWDINANVHYLFDVADKFKVYPLAGLGYTNWSYKYEYAGAPVVEGSDGRLAVNLGGGVEYELTKNLNVNAEAKYQIISNYNQLVLGVGVAYKF

SEQ ID NO: 22 - protein, 84 aa, Prevotella sp.
MHFYCTKSSLDTMSERYVKRMIAKLASQGKTVISIAHRFSTIMDAKHIILLAKGKVVAEGTHQELLKTSEDYRKLWSDQNDEID

SEQ ID NO: 23 - protein, 254 aa, Prevotella sp.
MKNVYFLSDAHLGSLAIAHRRTQERRLVRFLDSIKHKASAVYLLGDMFDFWDEYKYVVPKGFTRFLGKVSELTDMGVEVHFFTGNHDLWTYGYLEEECGVILHRKPVTMEIYGKVFYLAHGDGLGDPDPMFQFLRKVFHNRVCQRLLNFFHPWWGMQLGLNWAKKSRLKRADGKEMPYLGEDKEYLVRYTKDYMRSHKDIDYYIYGHRHIELDLTLSGKVRMLILGDWIWQFTYAVFDGEHMFLEEYIEGESKP

SEQ ID NO: 24 - protein, 532 aa, Prevotella sp.
MNSKQNDNYDVIIIGGGITGAGTARDCALRGLKVLLVEKFDFTNGATGRNHGLLHSGARYAVTDPESATECIKENMVLRRIAKHCIEETDGLFITLPEDDINYQKTFVEACARAGISANIISPEEALRLDPSVNPDLLGAVRVPDASVDPFHLTTANVLDARQHGADVLTYHEVVAILTSNGRVEGVRLRNNHTGEEIEKHAVLVINAAGIWGHDIAKMADIKINMFPAKGTLLVFGHRVNKMVINRCRKPANADILVPDDAVCVIGTTSDRVPYDTVDNLKITSEEVDTLIREGEKLAPSLATTRILRAYAGVRPLVAADNDPTGRSISRGIVCLDHEKRDGLTGMITITGGKMMTYRLMAEQATDLACKKLGINKTCETATTPLPGTAGKDSDNPHHTYSTAHKAAKGRQGNRVKEIDERTEDDRALICECEEVSVGEAKYAIEELHVHDLLNLRRRTRVGMGTCQGELCACRAAGVMCENGVKVDKAMTDLTKFINERWKGMRPVAWGSTLDEAQLTTIIYQGLCGLGI

SEQ ID NO: 25 - protein, 416 aa, Prevotella sp.
MRYDTIIIGGGLSGLTAGITLAKAGQKVCIVSAGQSSLHFHSGSFDLLGYDADGEVVTHPLQAIADLKAEHPYSKIGISNIEHLASQAKTLLCEAGISVMGNYEQNHYRVTPLGTLKPAWLTTEGYAMIDDPEILPWKKVELLNIQGFMDFPTQFIAENLRMMGVECQIKTFTTDELSTARQSPTEMRATNIAKVLANKDALSKVSERINAISGDPDALLLPAVLGFSNAESLDEMKQWIKKPVQYIATLPPSVSGVRTTILLKRLFAQAGGTLLIGDSATTGQFSGNHLVSITTDHLPDEKLYADHFILASGSFMSHGIRSNYAGVYEPVFKLDVDAAEKRDDWSVTNAFEAQPYMEFGVHTDKDFHATKDGKNIENLYAIGSVLSGHNSIKHADGTGVSLLTALYVAKKITGKG

SEQ ID NO: 26 - protein, 416 aa, Prevotella sp.
MAEGIQLKNISGNNLEQCLKCSICTAYCPVSAVEPKYPGPKQSGPDQERYRLKDSKFFDEALKMCLNCKRCEVACPSGVRIADIIQASRITYSTHRPIPRDIMLANTDFVGTMANMVAPIVNATLGLKPVKAVLHGVMGIDKHRTFPAYSSQKFETWYKRMAAKKQDSYSKHVSYFHGCYVNYNFPQLGKDLVKIMNAVGYGVHLLEKEKCCGVALIANGLSGQARRQGKVNIRSIRKAAEQNRIVLTTSSTCTFTMRDEYEHLLDIKTDDVRENITLATRFLYRLIEKGDIKLAFRKDFKMRTAYHSACHMEKMGWIIYSTELLKMIPGLELIMLDSQCCGIAGTYGFKKENYQRSQEIGEGLFKQIKELNPDCVSTDCETCKWQIEMSTGYEVKNPISILADALDVEETIKLNQ

SEQ ID NO: 27 - protein, 270 aa, Prevotella sp.
MMIKNIVLSIPISLIIYLNHLIMEYSMTTQFLMELIGTLILVLFGDGVCACVTLNKSKGQKAGWVVITIAWGLAVCMGVLVAGPYTGAHLNPAVSIGLAVAGMFPWSSVPYYIVAQMIGGFLGGLLVWFFYKDHYDATDDEAAKLGTFCTSPAIRNYKMNFLSEVIATLVLVFIIISFSVDGNTGDAEHFKFGLAALGPIPVTLLIIALGMSLGGTTGYAMNPARDLSPRLAHAVCMKGDNDWSYSWIPVLGPIIGAIIAGFCGAALLLV

SEQ ID NO: 28 - protein, 398 aa, Prevotella sp.
MSEKIIPSNEPAQAASEPIKASYTEYTVIPSQGYCQFVKCKKGDQPVVLKGLKEAYRERVLLRNALKREFKQCQRLNHPGIVRYQGLVDVEGYGLCIEEEYVDGRTLQAYLKESHTDDEKITIVNQIADALRYAHQQGVAHRNLKPSNILITKQGDHVKLIDFNVLSLDDVKPTADTTRFMAPELKDETMTADGTADIYSLGTIMKVMGLTLAYSEVIKRCCAFKRSDRYSDIDEFLADFNHDGSSFSMPKIGKGTVVIGFIAVVVIALAALAYNYGGALVDQVGKIDVTSIFKSDAETAPEDSAMVKSVEQNNNDSVADEAPATGKLAFMNTMKPALYKDLDRLFAKHSDDRAKLNRAIKVYYRGLIQANDTLDNEQRAELDRVFGNYVKQKKAALK

SEQ ID NO: 29 - protein, 62 aa, Prevotella sp.
MLVAQLFVGVLQAQKPVQNRRQAVGQSMERQGLVNVKAVVPSIKVALMYARTDNFCHRMALS

SEQ ID NO: 30 - protein, 440 aa, Prevotella sp.
MITGLVIIQLLIVLALIFIGARVGGIGLGIYGMIGVFILVYGFGLAPGSAPIDVMMIIVAVITAASALQASGGLEYLVGVAAKFLQKHPDHITYFGPITCWLFCVVAGTAHTSYSLMPIIAEIAQTNKIRPERPLSLSVIAASLGITCSPVSAATAALISQDLLGAKGIELGTVLMICIPTAFISILVAAFVENHIGKELEDDPEYKRRVAAGLINPEAACEEVQKAENEHDPSAKHAVWAFLFGVALVILFGFLPQLRPEGVSMSQTIEMIMMSDAALILLVGKGKVGDAVNGNIFKAGMNAVVAIFGIAWMGNTFYVGNEKILDAALSSMISSTPILFAVALFLLSIMLFSQAATVTTLYPVGIALGINPLLLIAMFPACNGYFFLPNYPTEVAAIDFDRTGTTRVGKYVINHSFQIPGFITTIVSILLGVLMVQFFR

SEQ ID NO: 31 - protein, 351 aa, Prevotella sp.
MRILKITFVTVLALVMSTVVFAQKPKIRIIATGGTIAGVSASATSSAYGAGQVGVQTLIDAVPQIKDIADVSGEQLVNIGSQDMNDEVWLKLAKRINDLLNKEGYDGVLITHGTDTMEETAYFLSLTVHTDKPVVMVGSMRPSTAISADGPANLYNGICTLVDPSSKGHGVMVCMNNELFEAKSVIKTHTTDVSTFKGGLYGEMGYVYNGKPYFLHKPVAKQGLTSEFNVDNLTSLPKVGIVYGYANCSPLPIQAFVNAKFDGIVLAGVGDGNFYKDVFDVALKAQNSGIQIVRSSRVPFGPTNLNGEVDDAKYHFVASLNLNPQKARVLLMLALTKTKDWQKIQQYFNEY

SEQ ID NO: 32 - protein, 574 aa, Prevotella sp.
MALACAMTMSASAQMGTNPKWLGDAIFYQIYPSSYMDTDGNGIGDLPGITQKLDYIKSLGVNAIWLNPVFESGWFDGGYDVIDFYKIDPRFGTNTDMVNLVKEAHKRGIKVCLDLVAGHTSTKCPWFKESANGDRNSRYSDYFIWTDSISEADKKEIAERHKEANPASSTHGRYVEMNAKRGKYYEKNFFECQPALNYGFAKPDPNQPWEQPVTAPGPQAVRREMRNIMAFWFDKGVDGFRVDMASSLVKNDWGKKEVSKLWNEMREWKDKNYPECVLISEWSDPAVAIPAGFNIDFMIHFGIKGYPSLFFDRNTPWGKPWPGQDISKDYKFCYFDKAGKGEVKEFVDNFSEAYNATKNLGYIAIPSANHDYQRPNIGTRNTPEQLKVAMTFFLTMPGVPFIYYGDEIGMKYQMDLPSKEGSNERAGTRTPMQWTSGPTAGFSTCNPSQLYFPVDTEKGKLTVEAQQNDPRSLLNYTRELTRLRHSQPALRGNGEWILVSKESQPYPMVYKRTSGGETVVVAINPSDKKVSANIAHLGKAKSLIMTGKASYKTGKTEDAVELNGVSAAVFKIAE

SEQ ID NO: 33 - protein, 244 aa, Prevotella sp.
MNIAVIFAGGSGLRMHTKSRPKQFLDLNGKPIIIYTLELFDNHPGIDAIVVACIESWIPFLEKQLRKFEINKVVKIVPGGESGQASIYNGLCAAEAYIKSKNVASEDTTVLIHDGVRPLITEETITDNINKVAEVGSCITCIPATETLVVKQHDGSLEIPSRADSLIARAPQSFLLSDILTAHRRAIDEKKNDFIDSCTMMSHYGYRLGTIIGPMENIKITTPTDFFVLRAMVKVHEDQQIFGL

SEQ ID NO: 34 - protein, 327 aa, Prevotella sp.
MTEKKSVSIVLCTYNGTKYLQEQLDSILAQTYPLHEIIIQDDGSTDNTWQILEKYEEKYPLIHIYHNEGTHGVNANFLSAMHRTTGDFIAIADQDDIWETDKIANQMTTIGNKLLCSGLTRPFSSDGSFAYFDNRPRNVSIFRMMFLGLPGHTMLFRRELLRMMPPVTHSFFNVSLYDAALSILAASHDSIAFCNKVLVNFRRHADATTYNDYSRSLPSWQNGLYELLWGLRHYHQARSIALPIYRGKLALMEGITTNYHDFIEAKAIMRLETQKGLWAFLRLQYLLTKNHQRLFQTSGGSFIKMIRAWLYPVMQLYMYHHALRRCK

SEQ ID NO: 35 - protein, 437 aa, Prevotella sp.
MESFIIEGGHRLSGTIAPQGAKNEALEVICATLLTTEEVIIRNIPNILDVNNLIKLLQDIGVKVKKLGANDFSFQADEVKLDYLESIDFVKKCSSLRGSVLMIGPLLGRFGKATIAKPGGDKIGRRRLDTHFLGFKNLGARFVRIEDRDVYEIQADKLVGDYMLLDEASVTGTANIIMSAVMAEGTTTIYNAACEPYIQQLCHLLNAMGAKITGIASNLITIEGVTSLHGAEHRILPDMIEVGSFIGMAAMVGDGVRIKDVSIPNLGLILDTFRRLGVQIIEDEDDLIIPRQDHYVIDSFIDGTIMTISDAPWPGLTPDLISVLLVVATQAQGSVLFHQKMFESRLFFVDKLIDMGAQIILCDPHRAVVVGHDHAKKLRAGRMSSPDIRAGIALLIAALTAEGTSRIDNIAQIDRGYENIEGRLNALGAKVQRVEIC

SEQ ID NO: 36 - protein, 772 aa, Prevotella sp.
MERSGNFYKAIRLGYILISILIGCMAYNSLYEWQEIEALELGNKKIDELRKEINNINIQMIKFSLLGETILEWNDKDIEHYHARRMAMDSMLCRFKATYPAERIDSVRHLLEDKERQMCQIVQILEQQQAINDKITSQVPVIVQKSVQEQPKKSKRKGFLGIFGKKEEAKPTVTTTMHRSFNRNMRTEQQAQSRRLSVHADSLAARNAELNRQLQGLVVQIDGKVQTDLQKREAEITAMRERSFIQIGGLTGFVILLLVISYIIIHRNANRIKRYKQETADLIERLQQMAKRNEALITSRKKAVHTITHELRTPLTAITGYAGLIQKNFNADKTGMYIRNIQQSSDRMREMLNTLLSFFRLDDGKEQPNFSTCRISSIAHTLESEFMPIAINKGLALTVTNHTDAVVLTDKERILQIGNNLLSNAIKFTENGAVSLTMGYDNGMLKLIVKDTGSGMTEEEQQRVFGAFERLSNAAAKDGFGLGLSIVQRIVTMLGGTIQLKSEKGKGSRFTVEIPMQSAEELPERINKTQIHHNRTLHDIVAIDNDKVLLLMLKEMYAQEGIHCDTCTNAAELMEMIRRKEYSLLLTDLNMPDINGFELLELLRTSNVGNSRIIPIIVTTASGSCNREELLERGFSDCLLKPFSISELMEVSDKCAMKGKQNEKPDFSSLLSYGNESVMLDKLIAETEKEMQSVRDGEQRKDFQELDALTHHLRSSWEILRADQPLRELYKQLHGSAVPDYEALNNAVTAVLDKGSEIIRLAKEERRKYENG

SEQ ID NO: 37 - protein, 321 aa, Prevotella sp.
MKRSRFYITVGLILSLTLLMSACGQKKAKDGRTDTPTSGTIKFASDESFSPIVEELLQNYQFRYPQAHLLPIYTDDNTGMKLLLDQKVNLFITSHAMTKGEDAILRGKGPIPEVFPIGYDGIAFIVNRSNPDSCITVDDVKKILQGKIAKWNQLNPKNNRGSIEVVFDNKASATLHYVVDSILGGKNIKSENIVAAKNSKSVIDYVNKTPNAIGVIGSNWLNDHRDTTNTTFKKDVTVASISKATVASPSNSWQPYQAYLLDGRYPFVRTIYALLADPHKALPYAFANYIANPIGQMIIFKAGLLPYRGNINIREVEVKNQ

SEQ ID NO: 38 - protein, 198 aa, Prevotella sp.
MAGTKRIKTALISVFHKDGLDDLLKKLDEEGVQFLSTGGTQQFIESLGYECQKVEDVTSYPSILGGRVKTLHPKIFGGILARRDNEEDQKQMVEYTIPAIDLVIVDLYPFEQTVASGASAQDIIEKIDIGGISLIRAGAKNFKDVVIVPSKAEYPVLLQLLNTKGAETEIEDRKMFAERAFGVSSHYDTAIHSWFAAE

SEQ ID NO: 39 - protein, 472 aa, Prevotella sp.
MEEEKGGRIGQRPYILKIITERNYIIIIDMKKAKILLFVTALVAVLTSCGGGQKGLPTSDEYPVITIGASNAQLKTTYPATIKGVQDVEVRPKVSGFITKLNIHEGEYVHAGQVLFVIDNSTYQAAVRQAQAQVNSAQSAVAQAKANVVQANASLNSANAQAATSRLTYNNSQNLYNNKVIGDYELQSAKNTYETAQASVRQAQSGIASAQAAVKQAEAGVRQAQAMLSTAKDNLGFCYVKSPASGYVGSLPFKEDALVSASSAQPVTTISNTSTIEVYFSMTEADVLKLSRTDDGLSNAIKKFPAVSLLLADGSTYNHEGAIVKTSGMIDATTGTINVIARFPNPEHLLKSGGSGKIVIAKNNNRALLIPQEAVTQVQNKMFVYKVDAKDKVHYSEITVDPQNDGINYIVTSGLKMGERIVSKGVSSLEDGAKIKALTPAEYEEAIKKAEKLGENQSSASGFLKTMKGDSK

SEQ ID NO: 40 - protein, 292 aa, Prevotella sp.
MAKRRNKARSHHSLQVVTLCISTAMVLILIGMVVLTVFTSRNLSSYVKENLTVTMILQPDMSTEESAALCQRIRSLHYINSLNFISKEQALKEGTRELGANPAEFAGQNPFTGEIELQLKANYANNDSIKNIERELRTYRGVSDITYPQNLVESVNHTLGKISLVLLVIAILLTIVSFSLMNNTIRLSIYARRFSIHTMKLVGASWGFIRAPFLRRAVMEGLVSALLAIAVLGVGLCLLYDYEPDITKVLSWDVLVITAGVMLAFGVLIATFCSWLSVNKFLRMKAGDLYKI

SEQ ID NO: 41 - protein, 828 aa, Prevotella sp.
MKLSDLKTGETGVIVKVLGHGGFRKRIIEMGFIQGKQVEVLLNAPLRDPVKYKIMGYEVSLRHSEADQIEVISAEEARQLEQAKADNEPQQGALSNNIPDESDHALTPFELTDAANRKSKVINVALVGNPNCGKTSLFNFASGAHERVGNYSGVTVDAKVGRANYEGYEFHLVDLPGTYSLSAYSPEELYVRKQLVEKTPDVVINVIDASNLERNLYLTTQLIDMHVRMVCALNMFDETEQRGDNIDYQKISELFGIPMVPTVFTNGRGVKELFHQVIAVYEGKEDETSQFRHIHINHGHELEGGIKNIQEHLRAYPDICQRYSTRYLAIKLLEHDKDVEELIKPLKDSDEIFKHRDIAAQRVKEETGNESETAIMDAKYGFIHGALEEADYSTGQKKDTYQTTHFIDQILTNKYFGFPIFFLILFIMFTATFVIGQYPMDWIDGGVSWLGDFISSNMPDGPVKDMLVDGIIGGVGAVIVFLPQILILYFFISYMEDSGYMARAAFIMDKLMHKMGLHGKSFIPLIMGFGCNVPAVMATRTIESRRSRLVTMLILPLMSCSARLPIYVMITGSFFALKYRSLAMLSLYVIGILMSVIMSRVFSRFLVKGEDTPFVMELPPYRFPTWKAIGRHTWEKGKQYLKKMGGIILVASIIVWALGYFPLPDKPDMGQQERQEHSFIGQIGHAVEPVFRPQGFNWKLDVGLLAGVGAKEIVASTMGVLYSNDDSFKDDNSFSSEGGKYVKLHKQITQDVANLHGVSYNEAEPIATLTAFCFLLFVLLYFPCIATIAAIKGETGSWGWALFAAGYTTLLAWVVSAIVFQVGMLFIG

SEQ ID NO: 42 - protein, 566 aa, Prevotella sp.
MKKNLLKAVLPASLALFAVTFGSCSQDGQLTGTKEDTGERVLDNTREIQNYLRTLPLAPMMSRASDPVPSDDGTTVPVDEGTSKTEEKGVLNGIPGSWVKTTRRYKMTQAFDESFLFDPTSDIVYPGCVLKGGTIANGTYAIITSHETGDVTFSINLSPANPQEARETSATVHNIRKSEYQEVWNKWANMQWKESPITTIESVEKINSQEELATKLGVAVNSPVANGSLNFGFNFNKKKNHILARLIQKYFSVSTDAPKKGNIFESIDKEALDGYQPVYISNINYGRIIYLSVESDEDEKVVDEAINFAMNQIKGVDVSVSADQSLHYRKVLANCDIRITVLGGGQTIQKEVLKGDIDSFQRFLNADIPMEQMSPISFSLRYAVDNSQARVVTSNEFTVTQRDFVPEFKKVRMQLQVLGFSGTNTGPFPNLDREAGLWGSISLSLNGQDNELVKISQSNPFFFNYREKKETMHPIGFGGIVTVEFDKDPNESLEDFVDHQKMTFVSDLHSTRSIYNYNFGRTTFTHTLGTLYTKYKGDDPIFVLESNNKNVKIHTYVKVLDMKFFN

SEQ ID NO: 43 - protein, 922 aa, Prevotella sp.
MTKFIYAMSLFLLAAISIKAQPIQKTSGCLLHGSVVSSTDATAIAGATVRLYQLKKLVGGTVSDASGNFDVKCPSSGSLQLRITAVGFKEVDTTLNVPTVTPLSIYMRAGKHAMDEVTVTASEKRGMTSTTVIGQTAMEHLQPSSFADLLALLPGGMTKIPALGSANVITLREAGPPSSQYATSSLGTKFVIDGQAIGTDANMQYIAGSFQGDADNSRNHVSYGVDMREIPTDNIEKVEVVRGIPSVKYGELTSGLINITRKRSQSPLLLRLKADEYGKLVSVGKGFLLSGKWNLNVDGGLLDARKEPRNRFETYRRLTFSARLRRKWNLGERYVLEWSGATDYSLNIDNVKTDPEIQIHREDSYRSSYLKMGMNHRLLLRRKALVGLQSVSLAYSASLASDRIHQTEAVALQRDYVVPLAYEGGEYDGLFLPMQYLCDYRVEGKPFYSTLRGETEWLARTSFISHHITAGGEFLLNKNYGRGQIFDITKPLHASTARRPRSYKDIPATDILSFYAEDKATMPIGKHQLTVMAGLRTTQMLNIPASYAVHGKLFTDTRVNVQWDFPSFLGFKSFVSGGLGMMTKMPTVLDLYPDYVYKDITEMNYWDIRPAYKRIHIRTYKLNQVNPDLRPARNKKWEIRLGMDKGAHHFSVTYFHEDMKDGFRSTTTMRPFIYKRYDTSVINPSALTGPPSLASLPVVTDTLLDGYGRTENGSRITKQGIEFQYSSPRIPVIQTRITVNGAWFRTLYENSIPLFRSAPNVVVGTVAIADRYAGYYMSTDKYDKQIFTSNFIFDSYVDKLGLILSATAECFWMSNTKRPATSSTPMGYMDITGTVHPYVEADQSDPYLRWLVLTGTAGQDMDYRERSYMLVNFKATKRFGRHLSLSFFADRVFYVAPDYEVNGFIVRRTFSPYFGMEIGLKI

SEQ ID NO: 44 - protein, 257 aa, Prevotella sp.
MLIDFKKVNIYQDERLILKDIDFQATEGEFIYLIGRVGSGKSSLLKTFYGELDIDQEDAEKAEVLGESVLDIKQKRIPALRRQMGIIFQDFQLLHDRSVAKNLKFVLQATGWKDKEKIKQRIKEVLEQVGMIDKAAKMPSELSGGEQQRIAIARAFLNNPKIILADEPTGNLDPETASNIVSILKDTCKNGTTVIMSTHNINLLSQFPGKVYRCMEQALVPVTNEAQTKDLEEDSTSVEPLIEPVLEEEAQAEDSKE

SEQ ID NO: 45 - protein, 508 aa, Prevotella sp.
MFENQPKALYALALANTGERFGYYTMIAVFALFLRANFGLEPGTAGLIYSIFLGLVYFLPLIGGIMADKFGYGKMVTIGIIVMFAGYLFLSVPLGGGTVAFGAMLAALLLISFGTGLFKGNLQVMVGNLYDTPELASKRDSAFSIFYMAINIGALFAPTAAVKIKEWAETSLGYAGNDAYHFSFAVACVSLIVSMGIYYAFRSTFKHVEGGTKKTEKAAAAAVEELTPQQTKERIVALCLVFAVVIFFWMAFHQNGLTLTYFADEFVSPTSTGVQSMAFDVVNLVMIVFIVYSIMALFQSKTTKAKGIACAVILAAIAVLAYKYMNVNGQVEVSAPIFQQFNPFYVVALTPISMAIFGSLAAKGKEPSAPRKIAYGMIVAGCAYLLMVLASQGLLTPHEQKLAKAAGETVPFASANWLIGTYLVLTFGELLLSPMGISFVSKVAPPKYKGAMMGGWFVATAIGNILVSVGGYLWGDLSLTVVWTVFIVLCLVSASFMFLMMKRLEKVA

SEQ ID NO: 46 - protein, 492 aa, Prevotella sp.
MKKILIFVAGLCMSLAASAQIQRPKLVVGLVVDQMRWDYLYYYYNEYGTDGLRRLVDNGFSFENTHINYAPTVTAIGHSSVYTGSVPAITGIAGNYFFQDDKNVYCCEDPNVKSVGSDSKEGQMSPHRLLASTIGDELQISNDFRSKVIGVALKDRASILPAGHAADAAYWWDTSAGHFVTSTFYTDHLPQWVIDFNEKNHTAPNFNIKTSTQGVTMTFKMAEAALKNENLGKGKETDMLAVSISSTDAIGHVYSTRGKENHDVYMQLDKDLAHFLKTLDEQVGKGNYLLFLTADHGAAHNYNYMKEHRIPAGGWDYRQSVKDLNGYLQGKFGIAPVMAEDDYQFFLNDSLIAASGLKKQQIIDESVEYLKKDPRYLYVFDEERISEVTMPQWIKERMINGYFRGRSGEIGVVTRPQVFGAKDSPTYKGTQHGQPFPYDTHIPFLLYGWNVKHGATTQQTYIVDIAPTVCAMLHIQMPNGCIGTARNMALGN

SEQ ID NO: 47 - protein, 1138 aa, Prevotella sp.
MDRQVFQTDSRQRWNRFKWTLRVLITIAILLGVVFVAMFALEGSPQMPFRHDYRSVVSASEPLLKDNKRAEVYKSFRDFFKEQKMHSNYAKVAARQHRFVGHTDNVTQKYIKEWTDPRMGIRSAWYVNWDKHAYISLKNNLKNLNMVLPEWYFINPKTDRIEARIDQRALKLMRRAHIPVLPMLTNNYNSAFRPEAIGRIMRDSTKRMGMINELVAACKHNGFAGINLDLEELNINDNALLVTLVKDFARVFHANGLYVTQAVAPFNEDYDMQELAKYDDYLFLMAYDEYNAGSQAGPVSSQRWVEKATDWAAKNVPNDKIVLGMATYGYNWAQGQGGTTMSFDQTMATALNAGAKVNFNDDTYNLNFSYQDEDDGTLHQVFFPDAVTTFNIMRFGATYHLAGFGLWRLGTEDSRIWKYYGKDLSWESAARMPIAKIMQLSGTDDVNFVGSGEVLNVTSEPHAGRIGIVLDKDNQLIIEERYLSLPATYTVQRLGKCKEKQLVLTFDDGPDSRWTPKVLSILKHYKVPAAFFMVGLQIEKNIPIVKDVFNQGCTIGNHTFTHHNMIENSDRRSFAELKLTRMLIESITGQSTILFRAPYNADADPTDHEEIWPMIIASRRNYLFVGESIDPNDWQQGVTADQIYKRVLDGVHQEYGHIILLHDAGGDTREPTVTALPRIIETLQREGYQFISLEKYLGMSRQTLMPPIKKGKEYYAMQANLSLAELIYHISDFLTALFLVFLVLGFMRLVFMYVLMIREKRAENRRNYAPIDPLTAPAVSIIVPAYNEEVNIVRTISNLKEQDYPSLKIYLVDDGSKDNTLQRVREVFENDDKVVIISKKNGGKASALNYGIAACSTDYIVCVDADTQLYKDAVSKLMKHFIADKTGKLGAVAGNVKVGNQRNMLTYWQAIEYTTSQNFDRMAYSNINAITVIPGAIGAFRKDVLEAVGGFTTDTLAEDCDLTMSINEHGYLIENENYAVAMTEAPESLRQFIKQRIRWCFGVMQTFWKHRASLFAPSKGGFGMWAMPNMLIFQYIIPTFSPIADVLMLFGLFSGNASQIFIYYLIFLLVDASVSIMAYIFEHESLWVLLWIIPQRFFYRWIMYYVLFKSYLKAIKGELQTWGVLKRTGHVKGAQTIS

SEQ ID NO: 48 - protein, 508 aa, Prevotella sp.
MSQINGRISQIIGPVIDVYFDTKGENPEKVLPNIYDALRVKKADGQDLIIEVQQQIGEDTVRCVAMDNTDGLQRGLEVVPTGSPIVMPAGEQIKGRMMNVIGQPIDGMSALQMEGAYPIHREAPKFEDLSTHKEMLQTGIKVIDLLEPYMKGGKIGLFGGAGVGKTVLIMELINNIAKGHNGYSVFAGVGERTREGNDLIRDMLESGVIRYGEKFRKAMDEGKWDLSLVDSEELQKSQATLVYGQMNEPPGARASVALSGLTVAEEFRDHGGKNGEAADIMFFIDNIFRFTQAGSEVSALLGRMPSAVGYQPTLASEMGAMQERITSTKHGSITSVQAVYVPADDLTDPAPATTFTHLDATTELSRKITELGIYPAVDPLGSTSRILDPLIVGKEHYDCAQRVKQLLQKYNELQDIIAILGMDELSDDDKLVVNRARRVQRFLSQPFTVAEQFTGVKGVMVPIEETIKGFNAILNGEVDDLPEQAFLNVGTIEDVKEKAKQLLEATKA

SEQ ID NO: 49 - protein, 984 aa, Prevotella sp.
MNPIYKIITSILFCVLSINTMAQDLTGHVTSKADDKPIAYATVTLKENRLYAFTDEKGNYTIKNVPKGKYTVVFSCMGYASQTVVVMVNAGGATQNVRLAEDNLQLDEVQVVAHRKKDEITTSYTIDRKTLDNQQIMTLSDIAQLLPGGKSVNPSLMNDSKLTLRSGTLERGNASFGTAVEVDGIRLSNNAAMGETAGVSTRSVSASNIESVEVVPGIASVEYGDLTNGVVKVKTRRGSSPFIVEGSINQHTRQIALHKGVDLGGNVGLLNFSIEHARSFLDAASPYTAYQRNVLSLRYMNVFMKKSLPLTLEVGLNGSIGGYNSKADPDRSLDDYNKVKDNNVGGNIHLGWLLNKRWITNVDLTAAFTYADRLSESYTNESSNATQPYIHTLTEGYNIAEDYDRNPSANIILGPTGYWYLRGFNDSKPLNYSLKMKANWSKAFGKFRNRLLVGGEWTSSMNRGRGTYYADMRYAPSWREYRYDALPSLNNIAIYAEDKLSMDVNERQNAELTAGIREDITSIPGSEYGSVGSFSPRMNARYVFRFGQNSWLNSMTLHAGWGRSVKIPSFQVLYPSPSYRDMLAFASTSDADNRSYYAYYTYPSMARYNANLKWQRADQWDLGVEWRTKIADVSLSFFRSKVSNPYMATDVYTPFTYKYTSPAMLQRSGIAVADRRFSIDPQTGIVTVSDASGVKSPVTLGYEERNTYVTNTRYVNADALQRYGLEWIVDFKQIKTLRTQVRLDGKYYHYKAQDETLFADVPVGLNTRQSDGRLYQYVGYYRGGAATTTNYTANASASNGSVSGQVDLNATITTHIPKIRLIVALRLESSLYAFSRATSSRGYVVSSGNEYFGVPYDDKTENQTVIVYPEYYSTWDAPDVLIPFAEKLRWAETNDRGLFNDLAQLVVRTNYPYTLNPNRLSAYWSANLSVTKEIGRHVSVSFYANNFFNTLSQVHSTQTGLETSLFGSGYVPSFYYGLSLRLKI

SEQ ID NO: 50 - protein, 273 aa, Prevotella sp.
MERIDISVLMAVYKKDNPAFLRESLESIFSQTVEAAEVVLLEDGPLTDALYDVIKSYEAIYSTLKVVSYPENRGLGKTLNDGLLLCKYNLVARMDADDICKPNRLEMEYNWLKSHEDYDVIGSWVDEFTDNKTRVKSIRKVPEAYDEIKNYAQYRCPINHPTAMYRKAAVLAVGGYLTEYFPEDYFLWLRMLNNGSKFYNIQESLLWFRYSEETVAKRGGWAYACDEVRILVRMLKMGYIPFHVFCQSVVIRFTTRVMPLPIRQRLYNLIRKT

SEQ ID NO: 51 - protein, 507 aa, Prevotella sp.
MSQINGRISQIIGPVIDVYFDTKGENPEKVLPKIHDALRVKRANGQDLIIEVQQHIGEDTVRCVAMDNTDGLQRNLEVVPTGSPIVMPAGDQIKGRMMNVIGQPIDGMEALSMEGAYPIHREAPKFEDLSTHKEMLQTGIKVIDLLEPYMKGGKIGLFGGAGVGKTVLIMELINNIAKGHNGYSVFAGVGERTREGNDLIRDMLESGVIRYGEKFRKAMDEGKWDLSLVDQEELQKSQATLVYGQMNEPPGARASVALSGLTVAEEFRDHGGKNGEAADIMFFIDNIFRFTQAGSEVSALLGRMPSAVGYQPTLASEMGTMQERITSTKHGSITSVQAVYVPADDLTDPAPATTFTHLDATTELSRKITELGIYPAVDPLGSTSRILDPLIVGKDHYECAQRVKQLLQHYNELQDIIAILGMDELSDEDKLVVNRARRVQRFLSQPFTVAEQFTGVKGVMVPIEETIKGFNAILNGEVDDLPEQAFLNVGTIEDVKEKAKRLLEATK

SEQ ID NO: 52 - protein, 268 aa, Prevotella sp.
MPIGNGQKYQLTIINHTEIIMLIDYKKVNIYQDERLILKDVDFQAETGEFIYLIGRVGSGKSSLLKTIYGELDIDSEDAEKAVVLDESMPNIKRSRIPALRKQMGIIFQDFQLLHDRSVAKNLKFVLQATGWTSKQKIERRIEEVLAQVGMTDKKNKMPSELSGGEQQRIAIARALLNTPKIIIADEPTGNLDPETAANIVSILKDSCQAGTTVIMSTHNINLIDQFPGKVYRCHEGELHQLTDKKEVSELAEETAPVETIDEPEQND

SEQ ID NO: 53 - protein, 336 aa, Prevotella sp.
MKRNILLFICLATSILLLFGLNLTTGSVQIPFADILDILCGRFIGKESWEYIILENRLPQTLTAILCGASLSVCGLMLQTAFRNPLAGPDVFGISSGAGLGVALVMLLLGGTVSTSIFTVSGFLAILTAAFVGAIAVTALILFLSTLVRNSVLLLIVGIMVGYVSSSAVSLLNFFASEEGVKSYMVWGMGNFGAVSMNHIPLFSILCLIGIIASFLLVKPLNILLLGPQYAESLGISTRQIRNILLVVVGLLTAITTAFCGPISFIGLAIPHIARLLFRTENHQILLPGIVLSGAAIALLCNFICYLPGESGIIPLNAVTPLIGAPIIIYVIIQRR

SEQ ID NO: 54 - protein, 408 aa, Prevotella sp.
MKKYYPWVLVALLWFVALLNYMDRQMLSTMQEAMKVDIAELNHAEAFGALMAVFLWIYGIVSPFAGIIADRVNRKWLVVGSIFVWSAVTYLMGYAESFDQLYWLRAFMGISEALYIPAALSLIADWHEGKSRSLAIGIHMTGLYVGQAVGGFGATLAAMFSWHAAFHWFGIIGIVYSLVLLLFLKENPKHGQKSVLQGETKPSKNPFRGLSIVFSTWAFWVILFYFAVPSLPGWATKNWLPTLFANSLDIPMSSAGPMSTITIAVSSFIGVIMGGVISDRWVQRNLRGRVYTSAIGLGLTVPALMLLGFGHSLVSVVGAGLCFGIGYGMFDANNMPILCQFISSKYRSTAYGIMNMTGVFAGAAVTQVLGKWTDGGNLGNGFAILGGIVVLALVLQLSCLKPTTDNME

SEQ ID NO: 55 - protein, 694 aa, Prevotella sp.
MVTKKTTTKKAPVKKTSAKTTKVKEPSHIGLVKNDAYLAPYEDAIRGRHEHALWKMNQLTQNGKLTLSDFANGHNYYGLHQTADGWVFREWAPNATEIYLVGDFNGWNEQEAYQCHRIEGTGNWELTLPHDAMQHGQYYKMRVHWEGGEGERIPAWTQRVVQDEASKIFSAQVWAPAEPYVWEKKTFKPQTSPLLIYECHIGMAQDEEKVGTYNEFREKVLPRIIKDGYNAIQIMAIQEHPYYGSFGYHVSSFFAASSRFGTPEELKALIDEAHKNGIAVIMDIVHSHAVKNEVEGLGNLAGDPNQYFYPGERHEHPAWDSLCFDYGKDEVLHFLLSNCKYWLEEYHFDGFRFDGVTSMLYYSHGLGEAFCNYADYFNGHQDDNAICYLTLANCLIHEVNKNAVTIAEEVSGMPGLAAKFKDGGYGFDYRMAMNIPDYWIKTIKELPDEAWKPSSIFWEIKNRRSDEKTISYCESHDQALVGDKTIIFRLVDADMYWHFRKGDETEMTHRGIALHKMIRLATIAAINGGYLNFMGNEFGHPEWIDFPREGNGWSHKYARRQWNLVDNEELCYHLLGDFDRKMLEVITSEKKFNETPIQEIWHNDGDQILAFSRGELVFVFNFSPSHSYSDYGFLVPEGSYNVVLNTDAREFGGFGFADDTVEHFTNSDPLYEKDHKGWLKLYIPARSAVVLRKK

SEQ ID NO: 56 - protein, 448 aa, Prevotella sp.
MKIDIERIKYFLTVGMFMKTEHSSKRRNMLIRQFQKFYLTVKFFFVRDHAASTAQLSFSTIMAIVPIASMIFAIANGFGFGQFLEKQFREMLSAQPEAATWLLKLTQSYLVHAKTGLFIGIGLMIMLYSVFSLIRTVETTFDNIWQVKDSRPISRIVIDYTALMFLVPISIIILSGLSIYFYSFVENLNGLRFLGTIASFSLRYLVPWAILTLMFIVLYVFMPNAKVKITKTVAPAMIASIAMLCLQAVYIHGQIFLTSYNAIYGSFAALPLFMLWILASWYICLFCAELCYFNQNLEYYECLIDTEDICHNDLLILCATVLSHICQRFANDQKPQTALQIKTETHIPIRVMTDILYRLKEVNLISENFSPTSDEVTYTPTHDTNNITVGEMIARLESTPASDFALLGFSPKKAWNHDIYDRVGSIREIYLNELKSINIKELISYSEN

SEQ ID NO: 57 - protein, 326 aa, Prevotella sp.
MMKRPSIARVVKVIICLLTPILLSFSGIGDNDIDKKKSTSKEVDDTLRIVITGDLLLDRGVRQKIDMAGVDALFSPTIDSLFHSSNYVIANLECPVTKIRERVFKRFIFRGEPEWLPTLRRHGITHLNLANNHSIDQGRNGLLDTQEQIKKAGMIPIGAGKNMEEAAEPVLISTSPRHVWVISSLRLPLENFLYLPQKPCVSQESIDSLIMRVKRLRATDKNCYILLILHWGWEHHFRATPQQREDAHKLIDAGADAIVGHHSHTLQTIETYRGKPIYYGIGNFIFDQRKPMNSRACLVELSITAEKCKAKALPIEIKNCTPYLSK

SEQ ID NO: 58 - protein, 259 aa, Prevotella sp.
MILLSFDTEEFDVPREHGVDFSLEEGMKVSIEGTNRILDILKANNVCATFFCTGNFAELAPEVMERIKNEGHEVACHGVDHWQPKPEDVFRSKEIIERVTGVKVAGYRQPRMFPVSDEDIEKAGYLYNSSLNPAFIPGRYMHLTTSRTWFMQGKVMQIPASVSPHLRIPLFWLSMHNFPEWFYLRLVRQVLRHDGYFVTYFHPWEFYDLKSHPEFKMPFIIKNHSGHELEQRLDRFIKAMKADKQEFITYVDFVNRQKK

SEQ ID NO: 59 - protein, 252 aa, Prevotella sp.
MAKNISFTIKYWKQNGPQDQGHFDTHEMKNIPDDTSFLEMLDILNEELIAAGDEPFVFDHDCREGICGMCSLYINGTPHGKTERGATTCQLYMRRFNDGDVITVEPWRSAGFPVIKDCMVDRTAFDKIIQAGGYTTIRTGQAQDANAILISKDNADEAMDCATCIGCGACVAACKNGSAMLFVSSKVSQLALLPQGKPEAAKRAKAMVAKMDEVGFGNCTNTRACEAVCPKNEKIANIARLNREFIKAKFAD

SEQ ID NO: 60 - protein, 398 aa, Prevotella sp.
MSENKLSTNEQAQTADAPVKASYTEYKVIPSQGYCMIVKCRKGDQTVVLKTLKEEYRERVLLRNALKREFKQCQRLNHSGIVRYQGLVEVDGYGLCIEEEYVEGRTLQAYLKENHTDDEKIAIINQIADALRYAHQQGVIHRNLKPSNVLVTTQGDYVKLIDFSVLSPEDVKPTAETTRFMAPEMKDETLTADATADIYSLGTIMKVMGLTLAYSEVIKRCCAFKRSDRYSNVDELLADLNNEGSSFSMPKIGKGTVVLGLIIAVVIGIGALLYNYGGALIDQVGKIDVSSVFSSDAETAPEDTVKVNTAEQSDSLSTEAEAPAIGKLAFMNRMKPALYKDLDNIFEKNSADKAKLTKAIKTYYRGLIQANDTLDNEQRAEVDRVFGDYVKQKKAALN

SEQ ID NO: 61 - protein, 522 aa, Prevotella sp.
MRKYICLLLFYLFTFLPLSAQQGNDSPLRKLQLAEMAIKNFYVDSVNEQKLVEDGIRGMLEKLDPHSTYTDAKETKAMNEPLQGDFEGIGVQFNMIEDTLVVIQPVVNGPSQKVGILAGDRIVSVNDSTIAGVKMARIDIMKMLRGKKGTKVKLGVVRRGVKGVLTFVVTRAKIPVHTINASYMIRPNVGYIRIESFGMKTHDEFMSAVDSLKKKGMKTLLLDLQDNGGGYLQSAVQISNEFLKNNDMIVYTEGRRARRQNFKAIGNGRLQDVKVYVLVNELSASAAEIVTGAIQDNDRGTVVGRRTFGKGLVQRPFDLPDGSMIRLTIAHYYTPSGRCIQKPYTKGDLKDYEMDIEKRFKHGELTNPDSIQFSDSLKYYTIRKHRVVYGGGGIMPDNFVPLDTTKFTRYHRMLAAKSIIINAYLKYADANRQALKAQYSSFDAFNKGYVVPQSLLDEIVAEGKKEKIEPKDAAELKATLPNIALQIKALTARDIWDMNEYFRVWNTQSDIVNKAVALATGK

SEQ ID NO: 62 - protein, 348 aa, Prevotella sp.
MKLTEQRSSMLHGVLLITLFACAAFYIGDMGWVKALSLSPMVVGIILGMLYANSLRNNLPDTWVPGIAFCGKRVLRFGIILYGFRLTFQDVVAVGFPAIIVDAIIVSGTILLGVLVGRLLKMDRSIALLTACGSGICGAAAVLGVDGAIRPKPYKTAVAVATVVIFGTLSMFLYPILYRAGIFDLSPDAMGIFAGSTIHEVAHVVGAGNAMGAAVSNSAIIVKMIRVMMLVPVLLVIAFFVAKNVAERDDEAGGSRKINIPWFAILFLVVIGFNSLNLLPKELVDFINTLDTFLLTMAMSALGAETSIDKFKKAGFKPFLLAAILWCWLIGGGYCLAKYLVPVLGVAC

SEQ ID NO: 63 - protein, 833 aa, Prevotella sp.
MNKQFLLAALWLSPLGLYAHKANGIGAVTWKNEAPKERMIRGIDEDKTHQRFTLSGYVKDRNGEPLINATIYDLTTRQGTMTNAYGHFSLTLGEGQHEIRCSYVGYKTLIETIDLSANQNHDIILQNEAQLDEVVVTTDLNSPLLKTQTGKLSLSQKDIKTEYALLSSPDVIKTLQRTSGVADGMELASGLYVHGGNGDENLFLLDGTPLYHTNHSLGLFSSFNADVVKNVDFYKSGFPARYGGRLSSVIDVRTADGDLYKTHGSYRIGLLDGAFHIGGPIRKGKTSYNFGLRRSWMDLLTRPAFAIMNHKSDNEDKLSMSYFFHDLNFKLTNIFNERSRMSLSVYSGEDRLDAKDEWHSNNSSGYNDVDIYVNRFHWGNFNAALDWNYQFSPKLFANFTAVYTHNRSTVSSSDEWRFTRPGEKEQLTLTSHGYRSSIDDIGYRAAFDFRPSPRHHIRFGQDYTYHRFQPQTYNRFDNYQTNSEAKADTIATHSYNKNVAHQLTFYAEDEMTLNEKWSLNGGVNADVFHISGKTFATLSPRLSMKFQPTERLSLKASYTLMSQFVHKIANSFLDLPTDYWVPTTARLHPMRSWQVAAGAYMKPNKHWLLSLEAYYKRSSHILQYSSWAGLEPPAANWDYMVMEGDGRSYGVELDADYNVSNLTLHGSYTLSWTQKKFDDFYDGWYYDKFDNRHKLTLTGRWNITKKIAAFAAWTFRTGNRMTIPTQYIGLPDVPAQEQGGLTFNSSDDNTLNFAYEKPNNVILPAYHRLDIGFDFHHTTKKGHERIWNLSFYNAYCHLNSLWVRVKIDSNNQMKIRNIAFIPVIPSFSYTFKF

SEQ ID NO: 64 - protein, 1133 aa, Prevotella sp.
MSKQVFQTDSRQRWSYFKWTLRVILTILSLLGIVFLAMFALEGSPQMPFRHDYRNAVTAASPYTKDNKTAKLYKSFRDFFKEKKMHNNYAKATIKKQRFIGKADSVTQKYFREWDDPRIGVRSAWYVNWDKHAYISLKNNIKHLNMVLPEWFFINPKTDKVEYRIDKQALRLMRRTGIPVLPMLTNNYNSDFHPEAIGRIMRDEKKRMALINEMVRTCRHYGFAGINLDLEELNIQDNDLLVELLKDFSRVFHANGLYVTQAVAPFNEDYNMQELAKYNDYLFLMAYDEHNIESQPGAVSSQRWVEKATDWAAKNVPNDKIVLGMATYGYDWANGEGGTTVSFDQTMAIAQDADAKVKFDDDTYNVNFSYQNTDDGKIHHVFFTDAATTFNIMRFGAEYHLAGYGLWRLGTEDKRIWRFYGKDMSWENVARMSVAKLMQLNGTDDVNFVGSGEVLEVTTEPHPGDISIRIDKDNRLISEEYYRALPSTYTIQRLGKCKDKQLVITFDDGPDSRWTPTVLSTLKKYNVPAAFFMVGLQMEKNLPLVKQVYEDGHTIGNHTFTHHNMIENSDRRSYAELKLTRMLIESVTGHSTILFRAPYNADADPTEHEEIWPMIVASRRNYLFVGESIDPNDWEPNVTSDQIYQRVIDGVHHEDGHIILLHDAGGSSRKPTLDALPRIIETLQHEGYQFISLEQYLGMGKQTLMPEINKGKAYYAMQTNLWLAEMIYHVSDFLTALFLVFLALGMMRLIFMYVLMIREKRAENRRNYAPIDAATAPAVSIIVPGYNEEVNIVRTITTLKQQDYPNLHIYFVDDGSKDHTLERVHEAFDNDDTVTILAKKNGGKASALNYGIAACRSEYVVCIDADTQLKNDAVSRLMKHFIADTEKRVGAVAGNVKVGNQRNMLTYWQAIEYTSSQNFDRMAYSNINAITVVPGAIGAFRKEVIEAVGGFTTDTLAEDCDLTMSINEHGYIIENENYAVALTEAPETLRQFVKQRIRWCFGVMQAFWKHRSSLFAPSKKGFGLWAMPNMLIFQYIIPTFSPLADVLMLIGLFTGNALQIFFYYLIFLVIDASVSIMAYIFEGERLWVLLWVIPQRFFYRWIMYYVLFKSYLKAIKGELQTWGVLKRTGHVKG

SEQ ID NO: 65 - protein, 292 aa, Prevotella sp.
MAKKRNKARSRHSLQVVTLCISTAMVLMLIGIVVLTGFTSRNLSSYVKENLTITMILQPDMNTEESAALCERIRTLHYINSLNFISKEQALKDGTKELGANPAEFAGENPFTGEIEVQLKANYANNDSIRNIVQQLRTYRGVSDITYPQSLVESVNQTLGKISLVLLVIAVLLTIISFSLINNTIRLSIYAHRFSIHTMKLVGGSWSFIRAPFLRRAVLEGLVSALLAIAVLGIGICLLYEKEPEITKLLSWDALIITAIVMLAFGVIIATFCAWLSVNKFLRMKAGDLYKI

SEQ ID NO: 66 - protein, 254 aa, Prevotella sp.
MKNIYFLSDAHLGSLAIDHRRTHERRLVRFLDSIKHKAAAVYLLGDMFDFWNEYKYVVPKGFTRFLGKISELTDMGVEVHFFTGNHDLWTYGYLEKECGVILHRKPITTEIYDKVFYLAHGDGLGDPDPMFRFLRKVFHNRFCQRLLNFFHPWWGMQLGLNWAKRSRLKRKDGKEVPYLGEDKEYLVQYTKEYMSTHKDIDYYIYGHRHIELDLTLSRKARLLILGDWIWQFTYAVFDGEHMFLEEYVEGESKP

SEQ ID NO: 67 - protein, 388 aa, Prevotella sp.
MVGLDVLCYFIHAKGREKECYFERIIYQITCHSRTKCYLCNIMKYSIIVPVFNRPDEVEELLESLLSQEEKDFEVVIVEDGSQIPCKEVCDKYADKLDLHYYSKENSGPGQSRNYGAERAKGEYLLILDSDVVLPKGYICAVSEELKREPADAFGGPDCAHESFTDTQKAISYSMTSFFTTGGIRGGKKKLDKFYPRSFNMGIRRDVYQELGGFSKMRFGEDIDFSIRIFKAGKRCRLFPEAWVWHKRRTDFRKFWKQVYNSGIARINLYKKYPESLKLVHLLPMVFTVGTALLVLMILFGLFLQLFPIINVFGSVFIMMGLMPLVLYSVIICVDSTMQNNSLNIGLLSIEAAFIQLTGYGCGFISAWWKRCVCGMDEFAAYEKNFYK

SEQ ID NO: 68 - protein, 435 aa, Prevotella sp.
MKIEKVHAREIMDSRGNPTVEVEVTLENGVMGRASVPSGASTGENEALELRDGDKNRFLGKGVLKAVENVNNLIAPALKGDCVLNQRAIDYKMLELDGTPTKSKLGANAILGVSLAVAQAAAKALNIPLYRYIGGANTYVLPVPMMNIINGGAHSDAPIAFQEFMIRPVGAPSEKEGIRMGAEVFHALAKLLKKRGLSTAVGDEGGFAPKFDGIEDALDSIIQAIKDAGYEPGKDVKIAMDCAASEFAVCEDGKWFYDYRQLKNGMPKDPNGKKLSADEQIAYLEHLITKYPIDSIEDGLDENDWENWVKLTSAIGDRCQLVGDDLFVTNVKFLEKGIKMGAANSILIKVNQIGSLTETLEAIEMAHRHGYTTVTSHRSGETEDTTIADIAVATNSGQIKTGSMSRTDRMAKYNQLIRIEEELGACAKYGYAKLK

SEQ ID NO: 69 - protein, 501 aa, Prevotella sp.
MKKLFTIAMLLGVTLGIHAQEVYSLQKCRELALQNNRQLKVSRMTVDVAENTRKAAKTKYLPRVDALAGYQHFSREISLLSDDQKNAFSNLGTNTFGQLGGQIGQNLTSLAQQGILSPQMAQQLGQLFSNVATPLTQVGNNIGQSINDAFRSNTKNVYAGGIVVNQPIYMGGAIKAANDMAAIGEQVAQNNISLKRQLVLYGVDNAYWLAISLKKKEALAIRYRDLAQKLNEDVKKMIREGVATRADGLKVEVAVNTADMQIARIQSGVSLAKMALCELCGLELNGDIPLSDEGDADLPPTPSTQFDNYTVSSSDTTGLNEARPELRLLQNAVDLSIQNTKLIRSLYMPHVLLTAGYSVSNPNLFNGFQKRFTDLWNIGITVQVPVWNWGENKYKVRASKTATTIAQLEMDDVRKKIDLEIEQNRLRLKDANKQLATSQKNMAAAEENLRCANVGFKEGVMTVTEVMAAQTAWQTSRMAIIDAEISVKLAQTGLQKALGGL

SEQ ID NO: 70 - protein, 577 aa, Prevotella sp.
MKRTFVTKMVKPIEENSLFFMFMLLVGAFTNVSHRNVFGYIELIADVYIICFLLSLCQRTIRQGLVIMLSSVIYVVAIIDTCCKTLFDTPITPTMLLLAQETTGREATEFFLQYLNLKLFFSAADIILFLAFCHIVMAVKKMKFSTSYLKQPFVAFVLMFTIFVGMALSIYDKVQLYTVKNLSGLEVAVTNGFAHLYHPVERIVYGLYSNHLIAKQVDGVIMANQQIKVDSCSFTSPTIVLVIGESANRHHSQLYGYPLPTTPYQLAMKNGKDSLAVFTNVVSPWNLTSKVFKQIFSLQSVDEKGDWSKYVLFPAVFKKAGYHVSFLSNQFPYGINYTPDWTNNLVGGFFLNHPQLNKQMFDYRNVTIHNYDEDLLNDYKEIISYKKPQLIIFHLLGQHFQYSLRCKSNMKKFGIKDYKRMDLTDKEKQTIADYDNATLYNDFVLNKIVEQFRNKDAIIVYLSDHGEDCYGKDVNMAGRLTEVEQINLKKYHEEFEIPFWIWCSPIYKQRHRKIFTETLMARNNKFMTDDLPHLLLYLAGIKTKDYCEERNVISPSFNNNRRRLVLKTIDYDKALYQ

SEQ ID NO: 71 - protein, 517 aa, Prevotella sp.
MFKNHPKGLLQAAFSNMGERFGYYIMNAVLALFLCSKFGLSDETSGLIASLFLAAIYVMSLVGGVIADRTQNYQRTIESGLVVMALGYVALSIPVLATPENNSYLLAFTIFALVLIAVGNGLFKGNLQAIVGQMYDDFETEAAKVSPERLKWAQGQRDAGFQIFYVFINLGALAAPFIAPVLRSWWLGRNGLTYDAALPQLCHKYINGTIGDNLGNLQELATKVGGNSADLASFCPHYLDVFNTGVHYSFIASVVTMLISLIIFMSSKKLFPMPGKKEQIVNVEYTDEEKASMAKEIKQRMYALFAVLGISVFFWFSFHQNGQSLSFFARDFVNTDSVAPEIWQAVNPFFVISLTPLIMWVFAYFTKKGKPISTPRKIAYGMGIAGFAYLFLMGFSLVHNYPSAEQFTSLEPAVRATMKAGPMILILTYFFLTVAELFISPLGLSFVSKVAPKNLQGLCQGLWLGATAVGNGFLWIGPLMYNKWSIWTCWLVFAIVCFISMVVMFGMVKWLERVTKS

SEQ ID NO: 72 - protein, 395 aa, Prevotella sp.
MQKKIKIGLLPRVIIAILLGLFLGYYLPDPAVRVFLTFNSIFSQFLGFMIPLIIIGLVTPAIAGIGKGAGKLLLATVAIAYVDTIVAGGLSYGTGTWLFPSMIASTGGAIPHIDKATELTPYFTINIPAMVDVMSSLVFSFIAGLGIAYGGLRTMENLFNEFKTVIEKVIEKAIIPLLPLYIFGVFLSMTHNGQARQVLLVFSQIIIVILVLHVLILIYEFCIAGAIVKHNPFRLLWNMLPAYLTALGTSSSAATIPVTLKQTVKNGVSEEVAGFVVPLCATIHLSGSAMKITACALTICMLTDLPHDPGLFIYFILMLAIIMVAAPGVPGGAIMAALAPLSSILGFNEEAQALMIALYIAMDSFGTACNVTGDGAIALAVNKFFGKKKETSILS

SEQ ID NO: 73 - protein, 208 aa, Prevotella sp.
MISVYSIKPQFQRVLTPILELLHRAKVTANQITLWACVLSLVIGILFWFAGDVGTWLYLCLPVGLLIRMALNALDGMMARRYNQITRKGELLNEVGDVVSDTIIYFPLLKYHPESLYFIVAFIALSIINEYAGVMGKVLSAERRYDGPMGKSDRAFVLGLYGVVCLFGINLSGYSVYIFGVIDLLLVLSTWIRIKKTLKVTRNSQTPE

SEQ ID NO: 74 - protein, 582 aa, Prevotella sp.
MKLSTILLSIMLGLSSSTMAQQKDVTIKLIETTDVHGSFFPYDFITRKPKSGSMARVYTLVEELRKKDGKDNVYLLDNGDILQGQPISYYYNYVAPEKTNIAASVLNYMGYDVATVGNHDIETGHKVYDKWFKELKFPILGANIIDTKTNKPYILPYYTIKKKNGIKVCVIGMLTPAIPNWLKESIWSGLRFEEMVSCAKRTMAEVKTQEKPDVIVGLFHSGWDGGIKTPEYDEDASKKVAKEVPGFDIVFFGHDHTPHSSIEKNIVGKDVICLDPANNAQRVAIATLTLRPKTVKGKRQYTVTKATGELVDVKELKADDAFIQHFQPEIDAVKAWSDQVIGRFENTIYSKDSYFGNSAFNDLILNLELEITKADIAFNAPLLFNASIKAGPITVADMFNLYKYENNLCTMRLTGKEIRKHLEMSYDLWCNTMKSPEDHLLLLSSTQNDAQRLGFKNFSFNFDSAAGIDYEVDVTKPDGQKVRILRMSNGEPFDENKWYTVAVNSYRANGGGELLTKGAGIPRDSLKSRIIWESPKDQRHYLMEEIKKAGVMNPQPNHNWKFIPETWTVPAAARDRKLLFGE

SEQ ID NO: 75 - protein, 826 aa, Prevotella sp.
MKLSELKTGETGVIVKVSGHGGFRKRIIEMGFIKGKTVEVLLNAPLQDPVKYKIMGYEVSLRHSEADQIEVLSDVKTHSVGNEEEQEDNQLEMDSTTYDSTDKELTPEKQSDAVRRKNHTINVALVGNPNCGKTSLFNFASGAHERVGNYSGVTVDAKVGRAEFDGYVFNLVDLPGTYSLSAYSPEELYVRKQLVDKTPDVVINVIDSSNLERNLYLTTQLIDMHIRMVCALNMFDETEQRGDHIDAQKLSELFGVPMIPTVFTNGRGVKELFRQIIAVYEGKEDESLQFRHIHINHGHEIENGIKEMQEHLKKYPELCHRYSTRYLAIKLLEHDKDVEQLVSPLGDSIEIFNHRDTAAARVKEETGNDSETAIMDAKYGFINGALKEANFSTGDKKDTYQTTHVIDHVLTNKYFGFPIFFLVLLVMFTATFVIGQYPMDWIEAGVGWLGEFISKNMPAGPVKDMIVDGIIGGVGAVIVFLPQILILYFFISYMEDCGYMSRAAFIMDRLMHKMGLHGKSFIPLIMGFGCNVPAVMATRTIESRRSRLITMLILPLMSCSARLPIYVMITGSFFALKYRSLAMLSLYIIGVLMAVAMSRLFSAFVVKGEDTPFVMELPPYRFPTWKAIGRHTWEKGKQYLKKMGGIILVASIIVWALGYFPLPDDPNMDNQARQEQSYIGRIGKAVEPVFRPQGFNWKLDVGLLSGMGAKEIVASTMGVLYSNDGSFSDDNGYSSETGKYSKLHNLITKDVATMHHISYEEAEPIATLTAFSFLLFVLLYFPCVATIAAIKGETGSWGWALFAAGYTTALAWIVSAVVFQVGMLFM

SEQ ID NO: 76 - protein, 437 aa, Prevotella sp.
MESFIIEGGHQLSGTIAPQGAKNEALEVICATLLTSEEVIIRNVPDILDVNNLIKLLQDIGVKVKKLAPNEFSFQADEVNLDYLESSDFVKKCSSLRGSVLMIGPLLGRFGKATIAKPGGDKIGRRRLDTHFLGFKNLGAHFGRVEDRDVYEIQADKLVGTYMLLDEASITGTANIIMAAVLAEGTTTIYNAACEPYIQQLCKMLNAMGAKISGIASNLITIEGVKELHSADHRILPDMIEVGSFIGIAAMIGDGVRIKDVSVPNLGLILDTFHRLGVQIIVDNDDLIIPRQDHYVIDSFIDGTIMTISDAPWPGLTPDLISVLLVVATQAQGSVLFHQKMFESRLFFVDKLIDMGAQIILCDPHRAVVVGHDNAKKLRAGRMSSPDIRAGIALLIAALTAQGTSRIDNIVQIDRGYENIEGRLNALGAKIQRAEVC

SEQ ID NO: 77 - protein, 244 aa, Prevotella sp.
MNIAVIFAGGSGLRMHTKSRPKQFLDLNGKPIIIYTLELFDNHPNIDAIVVACIESWIPFLEKQLRKFEINKVVKIIPGGKSGQESIYKGLCAAEEYAQSKGVSNEETTVLIHDGVRPLITEETITDNIKKVEEVGSCITCIPATETLIVKQADDALEIPSRADSFIARAPQSFRLIDIITAHRRSLAEGKADFIDSCTMMSHYGYKLGTIIGPMENIKITTPTDFFVLRAMVKVHEDQQIFGL

SEQ ID NO: 78 - DNA, 21 bp, Artificial Sequence
  source: /note=“Description of Artificial Sequence Synthetic primer”
gccctattcggagaactcaac

SEQ ID NO: 79 - DNA, 22 bp, Artificial Sequence
  source: /note=“Description of Artificial Sequence Synthetic primer”
gatcgccttcgacactcttaaa

SEQ ID NO: 80 - DNA, 24 bp, Artificial Sequence
  source: /note=“Description of Artificial Sequence Synthetic probe”
caccctattctacagcttggcggg

SEQ ID NO: 81 - DNA, 20 bp, Artificial Sequence
  source: /note=“Description of Artificial Sequence Synthetic primer”
gctcgaaagtccggttgatt

SEQ ID NO: 82 - DNA, 22 bp, Artificial Sequence
  source: /note=“Description of Artificial Sequence Synthetic primer”
acctgcggatggtaacttattg

SEQ ID NO: 83 - DNA, 24 bp, Artificial Sequence
  source: /note=“Description of Artificial Sequence Synthetic probe”
accggcaatcaggtactcatccat

(19) United States
(12) Patent Application Publication
(10) Pub. No.: US 2024/0024377 A1
(43) Pub. Date: Jan. 25, 2024

(54) COMPOSITIONS COMPRISING SPIRULINA COMPONENTS

(71) Applicant: Evelo Biosciences, Inc., Cambridge, MA (US)

(72) Inventors: Alexander BANK, Watertown, MA (US); David Emerson, Cambridge, MA (US)

(21) Appl. No.: 18/034,202

(22) PCT Filed: Oct. 29, 2021
(86) PCT No.: PCT/US2021/057216

§ 371 Date: Apr. 27, 2023

Related U.S. Application Data
(60) Provisional application No. 63/107,214, filed on Oct. 29, 2020.

(51) Int. Cl. A61K 35/74; A61K 35/618; A61K 9/20; A61K 9/48; A61K 38/45; A61K 38/16; A61K 31/555; A61K 31/015; A61K 35/748

(52) U.S. Cl. CPC A61K 35/74; A61K 35/618; A61K 9/2018; A61K 9/2054; A61K 9/2027; A61K 9/4858; A61K 9/485; A61K 9/4866; A61K 9/4816; A61K 38/45; A61K 38/168; A61K 31/555; A61K 31/015; A61K 9/2095; A61K 9/4833; A61K 35/748; C12Y 205/01062; A61K 2035/11

(57) ABSTRACT

Methods and compositions related to pharmaceutical agents, pharmaceutical compositions and solid dosage forms comprising at least one component of spirulina and bacteria or agents of bacterial origin are provided herein.

### CROSS-REFERENCE TO RELATED APPLICATION

This application claims the benefit of U.S. Provisional Application No. 63/107,214, filed Oct. 29, 2020, the entire content of which is incorporated herein by reference.

### BACKGROUND

The composition of a person's microbiome can play an important role in their health and well-being. Indeed, disruption of an individual's microbiome has been implicated in numerous diseases, including inflammatory bowel diseases, immune disorders, type 2 diabetes, neurodegenerative disorders, cardiovascular diseases, and cancers. Thus, microbiome modulation is an attractive therapeutic strategy for such diseases.

One way to modulate a person's microbiome is by orally administering to them one or more strains of beneficial bacteria. However, development of such therapies has been hindered by the fact that large-scale production of many bacterial strains has proven challenging, particularly for bacterial strains that require hemoglobin (or its derivatives, such as hemin) for growth. For example, commercial hemoglobin and its derivatives are typically purified from animal sources, such as from porcine blood, which results in purified hemoglobin being costly. Moreover, the animal sourcing of hemoglobin can raise ethical and/or religious objections among certain groups. Finally, GMP (good manufacturing practice)-grade hemoglobin is not easily sourced, making the large-scale manufacture of hemoglobin-dependent bacteria for pharmaceutical purposes particularly challenging.

### SUMMARY

As disclosed herein, spirulina can substitute for hemoglobin in supporting the growth of hemoglobin-dependent bacteria and has circumvented problems associated with the use of hemoglobin. This disclosure is based, in part, on the discovery that components of spirulina are present in pharmaceutical compositions and/or solid dosage forms comprising bacteria grown in growth media comprising spirulina and/or comprising agents (e.g., microbial extracellular vesicles, or mEVs) derived from bacteria grown in growth media comprising spirulina.

Thus, in certain aspects, provided herein are pharmaceutical compositions and/or solid dosage forms comprising bacteria (or components thereof, such as mEVs) and at least one component of spirulina (e.g., a nucleic acid spirulina component, a protein spirulina component, and/or a small molecule spirulina component). In certain aspects, provided herein are methods of making and/or using such pharmaceutical compositions and/or solid dosage forms.

In some aspects, provided herein are methods of determining the presence and/or amount of at least one component of spirulina in such a pharmaceutical composition or solid dosage form.

In certain aspects, provided herein is a pharmaceutical composition comprising: a pharmaceutical agent, wherein the pharmaceutical agent comprises (a) bacteria and/or microbial extracellular vesicles (mEVs); and (b) at least one component of spirulina.

In certain aspects, provided herein is a solid dosage form comprising: a pharmaceutical agent, wherein the pharmaceutical agent comprises (a) bacteria and/or microbial extracellular vesicles (mEVs); and (b) at least one component of spirulina.

This disclosure is also based, in part, on the discovery that components of spirulina are present in pharmaceutical agents comprising bacteria grown in growth media comprising spirulina and/or comprising agents (e.g., microbial extracellular vesicles, or mEVs) derived from bacteria grown in growth media comprising spirulina.

Thus, in certain aspects, provided herein are pharmaceutical agents comprising bacteria (or components thereof, such as mEVs) and at least one component of spirulina (e.g., a nucleic acid spirulina component, a protein spirulina component, and/or a small molecule spirulina component). In certain aspects, provided herein are methods of making and/or using such pharmaceutical agents.

In some aspects, provided herein are methods of determining the presence and/or amount of at least one component of spirulina in such a pharmaceutical agent.

In some aspects, provided herein are methods of determining the presence and/or amount of at least one component of spirulina in a pharmaceutical agent.

In some embodiments, the at least one component of spirulina comprises a spirulina nucleic acid. In some embodiments, the spirulina nucleic acid is spirulina DNA. In some embodiments, the spirulina DNA comprises a sequence encoding C-phycocyanin alpha subunit (cpcA). In some embodiments, the spirulina DNA comprises a sequence encoding chlorophyll a synthase (ChIG).

In some embodiments, the at least one component of spirulina comprises a spirulina protein. In some embodiments, the spirulina protein is phycocyanin.

In some embodiments, the at least one component of spirulina comprises a spirulina small molecule. In some such embodiments, the spirulina small molecule is a spirulina pigment. In some embodiments, the spirulina pigment is chlorophyllin. In some embodiments, the spirulina pigment is beta carotene.

In certain embodiments, the pharmaceutical agent comprises bacteria. In certain embodiments, the bacteria are hemoglobin-dependent bacteria (e.g., a species and/or strain of hemoglobin-dependent bacteria provided herein). In some embodiments, the bacteria are live, attenuated, or dead. In some embodiments, the bacteria are lyophilized bacteria. In some embodiments, the bacteria are irradiated (e.g., gamma irradiated).

In certain embodiments, the pharmaceutical agent comprises mEVs. In some embodiments, the mEVs are secreted mEVs (smEVs). In other embodiments, the mEVs are processed mEVs (pmEVs). In some embodiments, the mEVs are from hemoglobin-dependent bacteria (e.g., a species and/or strain of hemoglobin-dependent bacteria provided herein). In some embodiments, the mEVs are lyophilized mEVs.

In some embodiments, the bacteria (e.g., the bacteria in the pharmaceutical agent and/or in the pharmaceutical composition and/or in the solid dosage form and/or the bacteria from which the mEVs were derived) are hemoglobin-dependent bacteria. In some embodiments of the methods and compositions and agents provided herein, the hemoglobin-dependent bacteria can be any bacteria that require the presence of hemoglobin or a hemoglobin derivative for optimal growth (i.e., for optimal growth in the absence of spirulina or a component thereof provided herein). In some embodiments, the hemoglobin-dependent bacteria are bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, Turicibacter, or Veillonella. In some embodiments, the hemoglobin-dependent bacteria are of the genus Prevotella. In some embodiments, the hemoglobin-dependent bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oxalis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis. In some embodiments, the hemoglobin-dependent bacteria are Alistipes indistinctus, Alistipes shahii, Alistipes timonensis, Bacillus coagulans, Bacteroides acidifaciens, Bacteroides cellulosilyticus, Bacteroides eggerthii, Bacteroides intestinalis, Bacteroides uniformis, Collinsella aerofaciens, Cloacibacillus evryensis, Clostridium cadaveris, Clostridium cocleatum, Cutibacterium acnes, Eisenbergiella sp., Erysipelotrichaceae sp., Eubacterium hallii/Anaerobutyricum halii, Eubacterium infirmum, Megasphaera micronuciformis, Parabacteroides distasonis, Peptoniphilus lacrimalis, Rarimicrobium hominis, Shuttleworthia satelles, or Turicibacter sanguinis.

In some embodiments, bacteria are of the species Prevotella histicola. In some embodiments, the Prevotella histicola is Prevotella histicola Strain B (NRRL accession number B 50329) or Prevotella histicola Strain C (ATCC Deposit Number PTA-126140).

In some embodiments, the hemoglobin-dependent bacteria are a strain of the species Prevotella histicola. In some embodiments, the Prevotella histicola strain is a strain comprising at least 95%, at least 96%, at least 97%, at least 98%, or at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9% sequence identity) to a nucleotide sequence (e.g., genomic sequence, 16S sequence, CRISPR sequence) of the Prevotella Strain B 50329. In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) to the genomic sequence of the Prevotella Strain B 50329 (NRRL accession number B 50329). In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) of the 16S sequence of the Prevotella Strain B 50329 (NRRL accession number B 50329). In certain embodiments, the Prevotella histicola strain is Prevotella Strain B 50329 (NRRL accession number B 50329).

In some embodiments, the Prevotella histicola strain is a strain comprising at least 95%, at least 96%, at least 97%, at least 98%, or at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9% sequence identity) to a nucleotide sequence (e.g., genomic sequence, 16S sequence, CRISPR sequence) of the Prevotella Strain C (ATCC Deposit Number PTA-126140, deposited on Sep. 10, 2019). In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) to the genomic sequence of the Prevotella Strain C (PTA-126140). In certain embodiments, the Prevotella histicola strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) of the 16S sequence of the Prevotella Strain C (PTA-126140). In certain embodiments, the Prevotella histicola strain is Prevotella Strain C (PTA-126140).

In some embodiments, the hemoglobin-dependent bacteria are of the genus Fournierella. In some embodiments, the hemoglobin-dependent bacteria are Fournierella Strain A.

In some embodiments, the hemoglobin-dependent Fournierella strain is a strain comprising at least 95%, at least 96%, at least 97%, at least 98%, or at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9% sequence identity) to a nucleotide sequence (e.g., genomic sequence, 16S sequence, CRISPR sequence) of the Fournierella Strain B (ATCC Deposit Number PTA-126696, deposited on Mar. 5, 2020). In certain embodiments, the Fournierella strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) to the genomic sequence of the Fournierella Strain B (PTA-126696). In certain embodiments, the Fournierella strain is a strain that comprises at least 99% sequence identity (e.g., at least 99.1% sequence identity, at least 99.2% sequence identity, at least 99.3% sequence identity, at least 99.4% sequence identity, at least 99.5% sequence identity, at least 99.6% sequence identity, at least 99.7% sequence identity, at least 99.8% sequence identity, at least 99.9%, or 100% sequence identity) of the 16S sequence of the Fournierella Strain B (PTA-126696). In certain embodiments, the Fournierella strain is Fournierella Strain B (PTA-126696).

In some embodiments, the hemoglobin-dependent bacteria are of the genus Parabacteroides. In some embodiments, the hemoglobin-dependent bacteria are Parabacteroides Strain A. In some embodiments, the hemoglobin-dependent bacteria are Parabacteroides Strain B.

In some embodiments, the hemoglobin-dependent bacteria are of the genus Bacteroides. In some embodiments, the hemoglobin-dependent bacteria are Bacteroides Strain A.

In some embodiments, the hemoglobin-dependent bacteria are of the genus Allistipes. In some embodiments, the hemoglobin-dependent bacteria are Allistipes Strain A.

In certain aspects, provided herein is a solid dosage form comprising: (a) a pharmaceutical agent described herein (e.g., a pharmaceutical agent comprising at least one component of spirulina); and (b) at least one diluent, at least one lubricant, at least one glidant, and/or at least one disintegration agent.

In certain embodiments, the solid dosage form described herein comprises at least one diluent that has a total mass that is at least, about, or no more than, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total mass of the solid dosage form. In some embodiments, the at least one diluent has a total mass that is at least 10% and no more than 80% of the total mass of the solid dosage form. In other embodiments, the at least one diluent has a total mass that is at least 20% and no more than 40% of the total mass of the solid dosage form. In some embodiments, the at least one diluent comprises mannitol.

As used herein, the percent of mass of a solid dosage form is on a percent weight:weight basis (% w:w).

In certain embodiments, the solid dosage form described herein comprises at least one lubricant that has a total mass that is at least, about, or no more than, 0.01%, 0.03%, 0.04%, 0.05%, 0.06%, 0.07%, 0.08%, 0.09%, 0.1%, 0.2%, 0.3%, 0.4%, 0.6%, 0.7%, 0.8%, 0.9%, 1%, 2%, 3%, 4%, 5%, 6%, 7%, 8%, 9%, or 10% of the total mass of the solid dosage form. In some embodiments, the at least one lubricant has a total mass that is at least 0.1% and no more than 5% of the total mass of the solid dosage form. In some embodiments, the at least one lubricant comprises magnesium stearate.

In certain embodiments, the solid dosage form described herein comprises at least one glidant that has a total mass that is at least, about, or no more than, 0.001%, 0.003%, 0.004%, 0.005%, 0.006%, 0.007%, 0.008%, 0.009%, 0.01%, 0.02%, 0.04%, 0.05%, 0.06%, 0.07%, 0.08%, 0.09%, 0.1%, 0.2%, 0.3%, 0.4%, 0.5%, 0.6%, 0.8%, 0.9%, 1%, 2%, 3%, 4%, 5%, 6%, 7%, 8%, 9%, or 10% of the total mass of the solid dosage form. In some embodiments, the at least one glidant has a total mass that is at least 0.01% and no more than 2% of the total mass of the solid dosage form. In some embodiments, the at least one glidant comprises colloidal silicon dioxide.

In certain embodiments, the solid dosage form described herein comprises at least one disintegration agents, or certain combinations and/or amounts of disintegration agents, resulting in a decrease in the disintegration time of the composition (e.g., 2-fold, 3-fold, 4-fold, 5-fold, 6-fold, 7-fold, 8-fold, 9-fold) as compared to conventional solid dosage forms (e.g., solid dosage forms containing conventional amounts of disintegration agents). In certain embodiments, the solid dosage forms provided herein result in an increase in therapeutic efficacy and/or physiological effect as compared to a pharmaceutical product having conventional solid dosage forms.

In certain embodiments, the solid dosage form comprises a pharmaceutical agent (e.g., bacteria and/or an agent of bacterial origin, such as mEVs, a powder comprising bacteria and/or an agent of bacterial origin, such as mEVs) and one or more disintegration agents (e.g., one, two or three disintegration agents). In certain embodiments, the solid dosage form comprises a pharmaceutical agent (e.g., bacteria and/or an agent of bacterial origin, such as mEVs, a powder comprising bacteria and/or an agent of bacterial origin, such as mEVs) and three disintegration agents.

In some embodiments, the solid dosage form described herein comprises at least one disintegrant that has a total mass that is at least, about, or no more than, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total mass of the solid dosage form. In some embodiments, the at least one disintegration agent has a total mass that is at least 40% of the total mass of the solid dosage form.

In certain embodiments, the at least one disintegration agent comprises low-substituted hydroxypropyl cellulose (L-HPC, e.g., LH-B1), croscarmellose sodium (Ac-Di-Sol, e.g., Ac-Di-Sol SD-711), and/or crospovidone (PVPP, e.g., Kollidon, e.g., Kollidon CL-F). In some embodiments, the at least one disintegration agent comprises low-substituted hydroxypropyl cellulose (L-HPC, e.g., LH-B1), croscarmellose sodium (Ac-Di-Sol, e.g., Ac-Di-Sol SD-711), and crospovidone (PVPP, e.g., Kollidon, e.g., Kollidon CL-F).

In certain embodiments, the solid dosage forms provided herein comprise L-HPC. In some embodiments, the L-HPC is of grade LH-B1. In certain embodiments, the total L-HPC mass is at least 22%, 23%, 24%, 25%, 26%, 27%, 28%, 29%, 30%, 31%, 32%, 33%, 34%, 35%, 36%, 37%, 38%, 39%, 40%, 41%, or 42% of the total mass of the solid dosage form. In certain embodiments, the total L-HPC mass is no more than 22%, 23%, 24%, 25%, 26%, 27%, 28%, 29%, 30%, 31%, 32%, 33%, 34%, 35%, 36%, 37%, 38%, 39%, 40%, 41%, or 42% of the total mass of the solid dosage form. In certain embodiments, the total L-HPC mass is about 22%, 23%, 24%, 25%, 26%, 27%, 28%, 29%, 30%, 31%, 32%, 33%, 34%, 35%, 36%, 37%, 38%, 39%, 40%, 41%, or 42% of the total mass of the solid dosage form. In some embodiments, the L-HPC has a total L-HPC mass that is at least 22% and no more than 42% of the total mass of the solid dosage form. In certain embodiments, the total L-HPC mass is about 29% to about 35% of the total mass of the solid dosage form. In certain embodiments, the total L-HPC mass is about 32% of the total mass of the solid dosage form. In some embodiments, wherein the L-HPC is L-HPC of grade LH-B1.

In certain embodiments, the solid dosage forms provided herein comprise Ac-Di-Sol. In some embodiments, the Ac-Di-Sol is of grade SD-711. In certain embodiments, the total Ac-Di-Sol mass is at least 0.01%, 0.1%, 1%, 2%, 3%, 4%, 5%, 6%, 7%, 8%, 9%, 10%, 11%, 12%, 13%, 14%, 15%, or 16% of the total mass of the solid dosage form. In certain embodiments, the total Ac-Di-Sol mass is no more than 1%, 2%, 3%, 4%, 5%, 6%, 7%, 8%, 9%, 10%, 11%, 12%, 13%, 14%, 15%, or 16% of the total mass of the solid dosage form. In certain embodiments, the total Ac-Di-Sol mass is about 1%, 2%, 3%, 4%, 5%, 6%, 7%, 8%, 9%, 10%, 11%, 12%, 13%, 14%, 15%, or 16% of the total mass of the solid dosage form. In certain embodiments, the Ac-Di-Sol has a total Ac-Di-Sol mass that is at least 0.01% and no more than 16% of the total mass of the solid dosage form. In certain embodiments, the total Ac-Di-Sol mass is about 3% to about 9% of the total mass of the solid dosage form. In certain embodiments, the total Ac-Di-Sol (e.g., Ac-Di-Sol SD-711) mass is about 6% of the total mass of the solid dosage form.

In certain embodiments, the solid dosage forms provided herein comprise PVPP (crospovidone, e.g., Kollidon, e.g., Kollidon CL-F). In certain embodiments, the total PVPP mass is at least 5%, 6%, 7%, 8%, 9%, 10%, 11%, 12%, 13%, 14%, 15%, 16%, 17%, 18%, 19%, 20%, 21%, 22%, 23%, 24%, or 25% of the total mass of the solid dosage form. In certain embodiments, the total PVPP mass is no more than 5%, 6%, 7%, 8%, 9%, 10%, 11%, 12%, 13%, 14%, 15%, 16%, 17%, 18%, 19%, 20%, 21%, 22%, 23%, 24%, or 25% of the total mass of the solid dosage form. In certain embodiments, the total PVPP mass is about 5%, 6%, 7%, 8%, 9%, 10%, 11%, 12%, 13%, 14%, 15%, 16%, 17%, 18%, 19%, 20%, 21%, 22%, 23%, 24%, or 25% of the total mass of the solid dosage form. In certain embodiments, the PVPP has a total PVPP mass that is at least 5% and no more than 25% of the total mass of the solid dosage form. In certain embodiments, the total PVPP mass is about 12% to about 18% of the total mass of the solid dosage form. In certain embodiments, the total PVPP mass is about 15% of the total mass of the solid dosage form.

In certain embodiments, the total L-HPC mass plus the total Ac-Di-Sol mass plus the total PVPP mass is at least 35%, 40%, 45%, or 50% of the total mass of the solid dosage form. In certain embodiments, the total L-HPC mass plus the total Ac-Di-Sol mass plus the total PVPP mass is at least 40% of the total mass of the solid dosage form.

In certain embodiments, the solid dosage forms provided herein comprise: (i) L-HPC (e.g., L-HPC of grade LH-B1) having a total L-HPC mass that is at least 22% (e.g., at least 22%, 23%, 24%, 25%, 26%, 27%, 28%, 29%, 30%, 31%, 32%, 33%, 34%, 35%, 36%, 37%, 38%, 39%, 40%, 41%, or 42%) and no more than 42% (e.g., no more than 22%, 23%, 24%, 25%, 26%, 27%, 28%, 29%, 30%, 31%, 32%, 33%, 34%, 35%, 36%, 37%, 38%, 39%, 40%, 41%, or 42%) of the total mass of the solid dosage form; (ii) Ac-Di-Sol (e.g., Ac-Di-Sol of grade SD-711) having a total Ac-Di-Sol mass that is at least 0.01% (e.g., at least 0.01%, 0.1%, 1%, 2%, 3%, 4%, 5%, 6%, 7%, 8%, 9%, 10%, 11%, 12%, 13%, 14%, 15%, or 16%) and no more than 16% (e.g., no more than 1%, 2%, 3%, 4%, 5%, 6%, 7%, 8%, 9%, 10%, 11%, 12%, 13%, 14%, 15%, or 16%) of the total mass of the solid dosage form; and (iii) PVPP having a total PVPP mass that is at least 5% (e.g., at least 5%, 6%, 7%, 8%, 9%, 10%, 11%, 12%, 13%, 14%, 15%, 16%, 17%, 18%, 19%, 20%, 21%, 22%, 23%, 24%, or 25%) and no more than 25% (no more than 5%, 6%, 7%, 8%, 9%, 10%, 11%, 12%, 13%, 14%, 15%, 16%, 17%, 18%, 19%, 20%, 21%, 22%, 23%, 24%, or 25%) of the total mass of the solid dosage form. In some embodiments, the solid dosage form comprises: a total L-HPC mass is about 32% of the total mass of the solid dosage form; a total Ac-Di-Sol mass is about 6% of the total mass of the solid dosage form; and a total PVPP mass is about 15% of the total mass of the solid dosage form.

In some embodiments, the solid dosage forms provided herein comprise a pharmaceutical agent (e.g., bacteria and/or mEV) having a total mass that is at least, about, or no more than, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total mass of the solid dosage form. In some embodiments, the pharmaceutical agent has a total pharmaceutical agent mass that is at least 5% and no more than 65% of the total mass of the solid dosage form. In some embodiments, the pharmaceutical agent has a total pharmaceutical agent mass that is at least 5% and no more than 35% of the total mass of the solid dosage form. In some embodiments, the total pharmaceutical agent mass is about 25% of the total mass of the solid dosage form. In certain embodiments, the solid dosage forms described herein comprise tablets, capsules and/or minitablets (e.g., minitablets in capsules).

In some embodiments, the solid dosage form comprises a tablet. In some embodiments, the tablet is a 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, or 18 mm tablet.

In some embodiments, the solid dosage form comprises a minitablet. In some embodiments, the minitablet is a 1 mm minitablet, 1.5 mm minitablet, 2 mm minitablet, 3 mm minitablet, or 4 mm minitablet. In some embodiments, a plurality of minitablets are contained in a capsule (e.g., a size 0 capsule can contain about 31 to about 35 (e.g., 33) minitablets, wherein the minitablets are 3 mm in size). In some embodiments, the capsule is a size 00, size 0, size 1, size 2, size 3, size 4, or size 5 capsule. In some embodiments, the capsule comprises HPMC (hydroxyl propyl methyl cellulose) or gelatin.

In some embodiments, the solid dosage form is enterically coated (e.g., comprises an enteric coating; e.g., is coated with an enteric coating). In some embodiments, the enteric coating is a single enteric coating or more than one enteric coating. In some embodiments, the tablets or minitablets are coated with one layer of enteric coating or with two layers of enteric coatings (e.g., an inner enteric coating and an outer enteric coating). In some embodiments, the enteric coating comprises an inner enteric coating and an outer enteric coating, and the inner and outer enteric coatings are not identical.

In some embodiments, the enteric coating comprises a methacrylic acid ethyl acrylate (MAE) copolymer (1:1).

In some embodiments, the one enteric coating comprises methacrylic acid ethyl acrylate (MAE) copolymer (1:1) (such as Kollicoat MAE 100P).

In some embodiments, the one enteric coating comprises a Eudragit copolymer, e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

In some embodiments, the enteric coating comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate.

In some embodiments, the enteric coating comprises an anionic polymeric material.

In certain aspects, provided herein is solid dosage form comprising: (a) a pharmaceutical agent described herein (e.g., a pharmaceutical agent comprising at least one component of spirulina); and (b) at least one diluent, at least one lubricant, and/or at least one glidant.

In certain aspects, provided herein is a solid dosage form comprising (a) a pharmaceutical agent described herein (e.g., a pharmaceutical agent comprising at least one component of spirulina); and (b) a diluent. In certain embodiments, the total pharmaceutical agent mass is at least 5%, 10%, 15%, 20%, 25%, 30%, 35%, 40%, 45%, 50%, 55%, 60%, 65%, 70%, 75%, 80%, 85%, 90%, or 95% of the total mass of the solid dosage form. In some embodiments, the total pharmaceutical agent mass is no more than 95%, 90%, 85%, 80%, 75%, 70%, 65%, 60%, 55%, 50%, 45%, 40%, 35%, 30%, 25%, 20%, 15%, 10%, or 5% of the total mass of the solid dosage form.

In some embodiments, the total mass of the diluent is at least 1%, 5%, 10%, 15%, 20%, 25%, 30%, 35%, 40%, 45%, 50%, 55%, 60%, 65%, 70%, 75%, 80%, 85%, 90%, or 95% of the total mass of the solid dosage form. In some embodiments, the total mass of the diluent is no more than 95%, 90%, 85%, 80%, 75%, 70%, 65%, 60%, 55%, 50%, 45%, 40%, 35%, 30%, 25%, 20%, 15%, 10%, 5%, or 1% of the total mass of the solid dosage form. In some embodiments, the diluent comprises mannitol.

In certain embodiments, the solid dosage form provided herein comprises a lubricant. In certain embodiments, the total lubricant mass is at least 0.1%, 0.5%, 1%, 2%, 3%, 4%, or 5% of the total mass of the solid dosage form. In certain embodiments, the total lubricant mass is no more than 0.1%, 0.5%, 1%, 2%, 3%, 4%, or 5% of the total mass of the solid dosage form. In certain embodiments, the total lubricant mass is about 0.1%, 0.5%, 1%, 2%, 3%, 4%, or 5% of the total mass of the solid dosage form. In certain embodiments, the total lubricant mass is about 0.5% to about 1.5% of the total mass of the solid dosage form. In certain embodiments, the total lubricant mass is about 1% of the total mass of the solid dosage form. In some embodiments, the lubricant comprises magnesium stearate.

In certain embodiments, the solid dosage forms provided herein comprise a glidant. In some embodiments, the glidant is colloidal silicon dioxide. In certain embodiments, the total glidant mass is at least 0.01%, 0.1%, 0.2%, 0.3%, 0.4%, 0.5%, 0.7%, 0.8%, 0.9%, 1%, 1.5%, or 2% of the total mass of the solid dosage form. In certain embodiments, the total glidant mass is no more than 0.01%, 0.1%, 0.2%, 0.3%, 0.5%, 0.6%, 0.7%, 0.8%, 0.9%, 1%, 1.5%, or 2% of the total mass of the solid dosage form. In certain embodiments, the total glidant mass is about 0.01%, 0.1%, 0.2%, 0.3%, 0.4%, 0.5%, 0.6%, 0.7%, 0.8%, 0.9%, 1%, 1.5%, or 2% of the total mass of the solid dosage form. In certain embodiments, the total glidant mass is about 0.25% to about 0.75% of the total mass of the solid dosage form. In certain embodiments, the total glidant mass is about 0.5% of the total mass of the solid dosage form.

In certain embodiments, the solid dosage forms provided herein comprise: (i) a pharmaceutical agent having a total pharmaceutical agent mass that is at least 20% and no more than 55% of the total mass of the solid dosage form; (ii) a diluent (e.g., mannitol) having a total mass that is at least 45% and no more than 80% of the total mass of the solid dosage form; (iii) a lubricant (e.g., magnesium stearate) having a total mass that is at least 0.1% and no more than 5% of the total mass of the solid dosage form; and (iv) a glidant (e.g., colloidal silicon dioxide) having a total mass that is at least 0.01% and no more than 2% of the total mass of the solid dosage form.

In certain embodiments, the solid dosage forms provided herein comprise: (i) a pharmaceutical agent having a total pharmaceutical agent mass that is about 20% to about 50% of the total mass of the solid dosage form; (ii) a diluent (e.g., mannitol) having a total mass that is about 50% to 80% of the total mass of the solid dosage form; (iii) a lubricant (e.g., magnesium stearate) having a total mass that is about 1% of the total mass of the solid dosage form; and (iv) a glidant (e.g., colloidal silicon dioxide) having a total mass that is about 0.5% of the total mass of the solid dosage form.

In certain embodiments, the solid dosage forms provided herein comprise: (i) a pharmaceutical agent having a total pharmaceutical agent mass that is at least 5% and no more than 95% of the total mass of the solid dosage form; (ii) a diluent (e.g., mannitol) having a total mass that is at least 1% and no more than 95% of the total mass of the solid dosage form; (iii) a lubricant (e.g., magnesium stearate) having a total mass that is at least 0.1% and no more than 5% of the total mass of the solid dosage form; and (iv) a glidant (e.g., colloidal silicon dioxide) having a total mass that is at least 0.01% and no more than 2% of the total mass of the solid dosage form.

In certain embodiments, the solid dosage forms provided herein comprise: (i) a pharmaceutical agent having a total pharmaceutical agent mass that is about 8% to about 92% of the total mass of the solid dosage form; (ii) a diluent (e.g., mannitol) having a total mass that is about 5% to 90% of the total mass of the solid dosage form; (iii) a lubricant (e.g., magnesium stearate) having a total mass that is about 1% of the total mass of the solid dosage form; and (iv) a glidant (e.g., colloidal silicon dioxide) having a total mass that is about 0.5% of the total mass of the solid dosage form.

In certain embodiments, the solid dosage forms provided herein comprise: (i) a pharmaceutical agent having a total pharmaceutical agent mass that is about 30% to about 50% of the total mass of the solid dosage form; (ii) a diluent (e.g., mannitol) having a total mass that is about 45% to 70% of the total mass of the solid dosage form; (iii) a lubricant (e.g., magnesium stearate) having a total mass that is about 1% of the total mass of the solid dosage form; and (iv) a glidant (e.g., colloidal silicon dioxide) having a total mass that is about 0.5% of the total mass of the solid dosage form.

In certain embodiments, the solid dosage forms provided herein comprise: (i) a pharmaceutical agent having a total pharmaceutical agent mass that is about 50% of the total mass of the solid dosage form; (ii) a diluent (e.g., mannitol) having a total mass that is about 48.5% of the total mass of the solid dosage form; (iii) a lubricant (e.g., magnesium stearate) having a total mass that is about 1% of the total mass of the solid dosage form; and (iv) a glidant (e.g., colloidal silicon dioxide) having a total mass that is about 0.5% of the total mass of the solid dosage form. In certain embodiments, the solid dosage forms provided herein comprise: (i) a pharmaceutical agent having a total pharmaceutical agent mass that is about 13.51% of the total mass of the solid dosage form; (ii) a diluent (e.g., mannitol) having a total mass that is about 84.99% of the total mass of the solid dosage form; (iii) a lubricant (e.g., magnesium stearate) having a total mass that is about 1% of the total mass of the solid dosage form; and (iv) a glidant (e.g., colloidal silicon dioxide) having a total mass that is about 0.5% of the total mass of the solid dosage form.

In certain embodiments, the solid dosage forms provided herein comprise: (i) a pharmaceutical agent having a total pharmaceutical agent mass that is about 90.22% of the total mass of the solid dosage form; (ii) a diluent (e.g., mannitol) having a total mass that is about 8.28% of the total mass of the solid dosage form; (iii) a lubricant (e.g., magnesium stearate) having a total mass that is about 1% of the total mass of the solid dosage form; and (iv) a glidant (e.g., colloidal silicon dioxide) having a total mass that is about 0.5% of the total mass of the solid dosage form.

In certain embodiments, the solid dosage forms of a pharmaceutical agent as described herein comprise capsules. In some embodiments, the capsule is a size 00, size 0, size 1, size 2, size 3, size 4, or size 5 capsule. In some embodiments, the capsule is a size 0 capsule. In some embodiments, the capsule comprises HPMC (hydroxyl propyl methyl cellulose) or gelatin. In some embodiments, the capsule comprises HPMC (hydroxyl propyl methyl cellulose). In some embodiments, the capsule is banded. In some embodiments, the capsule is banded with an HPMC-based banding solution.

In some embodiments, the solid dosage form is enterically coated (e.g., comprises an enteric coating; e.g., is coated with an enteric coating).

In some embodiments, the solid dosage form is enteric coated to dissolve at pH 5.5.

In some embodiments, the enteric coating comprises a polymethacrylate-based copolymer. In some embodiments, the enteric coating comprises poly(methacrylic acid-co-ethyl acrylate).

In some embodiments, the enteric coating comprises a methacrylic acid ethyl acrylate (MAE) copolymer (1:1).

In some embodiments, the enteric coating comprises methacrylic acid ethyl acrylate (MAE) copolymer (1:1) (such as Kollicoat MAE 100P).

In some embodiments, the enteric coating comprises a Eudragit copolymer, e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

In some embodiments, the enteric coating comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate.

In some embodiments, the enteric coating comprises an anionic polymeric material.

The pharmaceutical agent can be a powder that comprises the bacteria and/or mEVs (such as smEVs and/or pmEVs) and a spirulina component, and, can comprise additional agents such as, e.g., cryoprotectant. For example, in some embodiments, the pharmaceutical agent is a lyophilized powder of bacteria and/or mEVs (such as smEVs and/or pmEVs) and a spirulina component that optionally, further comprises additional agents, such as a cryoprotectant.

In some embodiments, the pharmaceutical agent comprises bacteria and the dose of bacteria is about 1×10⁷ to about 2×10¹² (e.g., about 3×10¹⁰ or about 1.5×10¹¹) cells (e.g., wherein cell number is determined by total cell count, which is determined by Coulter counter), wherein the dose is per capsule or tablet or per total number of minitablets in a capsule.

In some embodiments, the pharmaceutical agent comprises bacteria and the dose of bacteria is about 1×10⁷ to about 1×10¹³, wherein the dose is per capsule or tablet or per total number of minitablets in a capsule. In some embodiments, the pharmaceutical agent comprises bacteria and the dose of bacteria is about 1×10⁹, about 3×10⁹, about 5×10⁹, about 1.5×10¹⁰, or about 5×10¹⁰ cells, wherein the dose is per capsule or tablet or per total number of minitablets in a capsule. In some embodiments, the pharmaceutical agent comprises bacteria and the dose of bacteria is about 8×10¹⁰ cells, wherein the dose is per capsule or tablet or per total number of minitablets in a capsule. In some embodiments, the pharmaceutical agent comprises bacteria and the dose of bacteria is about 1.6×10¹¹ cells, wherein the dose is per capsule or tablet or per total number of minitablets in a capsule. In some embodiments, the pharmaceutical agent comprises bacteria and the dose of bacteria is about 3.2×10¹¹ cells, wherein the dose is per capsule or tablet or per total number of minitablets in a capsule.

In some embodiments, the pharmaceutical agent comprises mEVs and the dose of mEVs is about 1×10⁵ to about 2×10¹² particles (e.g., wherein particle count is determined by NTA (nanoparticle tracking analysis)), wherein the dose is per capsule or tablet or per total number of minitablets in a capsule.

In some embodiments, the pharmaceutical agent comprises a powder comprising bacteria and/or mEVs and the dose of the pharmaceutical agent (e.g., a powder comprising bacteria and/or mEVs) is about 10 mg to about 3500 mg, wherein the dose is per capsule or tablet or per total number of minitablets in a capsule.

In some embodiments, the pharmaceutical agent comprises a powder comprising bacteria and/or mEVs and the dose of the pharmaceutical agent (e.g., a powder comprising bacteria and/or mEVs) is about 30 mg to about 1300 mg (by weight of bacteria and/or mEVs powder) (about 25, about 30, about 35, about 50, about 75, about 100, about 120, about 150, about 250, about 300, about 350, about 400, about 500, about 600, about 700, about 750, about 800, about 900, about 1000, about 1100, about 1200, about 1250, about 1300, about 2000, about 2500, about 3000, or about 3500 mg wherein the dose is per capsule or tablet or per total number of minitablets in a capsule.

In some embodiments, the pharmaceutical agent comprises bacteria and/or mEVs and the dose of pharmaceutical agent (e.g., bacteria and/or mEVs) is about 2×10⁶ to about 2×10¹⁶ particles (e.g., wherein particle count is determined by NTA (nanoparticle tracking analysis)), wherein the dose is per capsule or tablet or per total number of minitablets in a capsule.

In some embodiments, the pharmaceutical agent comprises bacteria and/or mEVs and the dose of pharmaceutical agent (e.g., bacteria and/or mEVs) is about 5 mg to about 900 mg total protein (e.g., wherein total protein is determined by Bradford assay or BCA), wherein the dose is per capsule or tablet or per total number of minitablets in a capsule.

In some aspects, the disclosure provides a method of preventing or treating a subject (e.g., human) (e.g., a subject in need of treatment), the method comprising administering to the subject a pharmaceutical composition provided herein. In some aspects, the disclosure provides a method of preventing or treating a subject (e.g., human) (e.g., a subject in need of treatment), the method comprising administering to the subject a solid dosage form provided herein. In some aspects, the disclosure provides use of a pharmaceutical composition for the treatment or prevention of a disease of a subject. In some aspects, the disclosure provides use of a solid dosage form for the treatment or prevention of a disease of a subject. In some aspects, the disclosure provides use of a pharmaceutical composition or a solid dosage form provided herein for the preparation of a medicament for treating a subject (e.g., human) (e.g., a subject in need of treatment).

In some embodiments, the pharmaceutical compositions and/or solid dosage forms provided herein treat a cancer, inflammation, autoimmunity, a metabolic condition, or a dysbiosis.

In some embodiments, the pharmaceutical compositions and/or solid dosage forms provided herein treat bacterial septic shock, cytokine storm and/or viral infection (such as a coronavirus infection, an influenza infection, and/or a respiratory syncytial virus infection).

In some embodiments, the pharmaceutical compositions and/or solid dosage forms provided herein decreases inflammatory cytokine expression (e.g., decreased IL-8, IL-6, IL-1β, and/or TNFα expression levels).

In certain aspects, provided herein are methods of preparing a solid dosage form, the method comprising (a) combining (i) a pharmaceutical agent provided herein (e.g., bacteria disclosed herein and/or an agent of bacterial origin, such as mEVs disclosed herein) (e.g., comprising at least one component of spirulina), and (ii) at least one diluent, at least one lubricant, at least one glidant, and/or at least one (e.g., one, two or three) disintegration agent, and (b) compressing the pharmaceutical composition into a solid dosage form. In some embodiments, the method further comprises the step of enterically coating the solid dosage form to obtain an enterically coated solid dosage form. In some embodiments, the solid dosage form is a tablet. In some embodiments, the solid dosage form is a minitablet.

In certain aspects, provided herein are methods of preparing a solid dosage form, the method comprising combining (i) a pharmaceutical agent (e.g., bacteria disclosed herein and/or an agent of bacterial origin, such as mEVs disclosed herein) (e.g., comprising at least one component of spirulina), and (ii) a diluent, lubricant, and/or glidant, e.g., into a pharmaceutical composition. In some embodiments, the method comprises blending. In some embodiments, the method further comprises loading the pharmaceutical composition into a capsule. In some embodiments, the capsule comprises HPMC.

In some embodiments, the method further comprises banding the capsule. In some embodiments, the capsule is banded with an HPMC-based banding solution.

In some embodiments, the method further comprises the step of enterically coating the solid dosage form to obtain an enterically coated solid dosage form. In some embodiments, the solid dosage form is a capsule.

In certain aspects, provided herein is a method of testing a pharmaceutical composition and/or a solid dosage form comprising bacteria (e.g., bacteria provided herein) and/or mEVs (e.g., mEVs provided herein) (e.g., a pharmaceutical composition provided herein and/or a solid dosage form provided herein), the method comprising performing an assay to detect the presence of a component of spirulina in the pharmaceutical composition and/or solid dosage form.

In certain aspects, provided herein is a method of testing a pharmaceutical agent comprising bacteria (e.g., bacteria provided herein) and/or mEVs (e.g., mEVs provided herein) (e.g., a pharmaceutical agent provided herein), the method comprising performing an assay to detect the presence of a component of spirulina in the pharmaceutical agent.

In certain embodiments, the component of spirulina comprises a spirulina nucleic acid. In some embodiments, the spirulina nucleic acid is spirulina DNA. In some embodiments, the spirulina DNA comprises a sequence encoding C-phycocyanin alpha subunit (cpcA). In some embodiments, the spirulina DNA comprises a sequence encoding chlorophyll a synthase (ChIG). In some embodiments, the assay to detect the presence of a component of spirulina is a nucleic acid amplification assay, a sequencing assay, and/or a microarray assay. In some embodiments, the assay to detect the presence of a component of spirulina is a polymerase chain reaction (PCR) assay, such as a quantitative polymerase chain reaction (qPCR) assay or digital PCR.

In certain embodiments, the component of spirulina is a spirulina protein. In some embodiments, the spirulina protein is phycocyanin. In some embodiments, the spirulina protein is detected using an antibody specific for the spirulina protein, HPLC or UPLC.

In certain embodiments, the component of spirulina comprises a spirulina small molecule. In some embodiments, the spirulina small molecule is a spirulina pigment. In some embodiments, the spirulina pigment is spirulina is chlorophyllin. In some embodiments, the spirulina pigment is spirulina is beta carotene. In some embodiments, the spirulina pigment is detected by HPLC or UPLC.

### DETAILED DESCRIPTION

In certain aspects, provided herein are pharmaceutical compositions and/or solid dosage forms comprising bacteria (or components thereof, such as mEVs) and at least one component of spirulina (e.g., a nucleic acid spirulina component, a protein spirulina component, and/or a small molecule spirulina component). In certain aspects, provided herein are methods of making and/or using such pharmaceutical compositions and/or solid dosage forms. In some aspects, provided herein are methods of determining the presence and/or amount of at least one component of spirulina in such pharmaceutical composition or solid dosage form.

In certain aspects, provided herein are pharmaceutical agents comprising bacteria (or components thereof, such as mEVs) and at least one component of spirulina (e.g., a nucleic acid spirulina component, a protein spirulina component, and/or a small molecule spirulina component). In certain aspects, provided herein are methods of making and/or using such pharmaceutical agents. In some aspects, provided herein are methods of determining the presence and/or amount of at least one component of spirulina in such pharmaceutical agent.

### Definitions

“Adjuvant” or “Adjuvant therapy” broadly refers to an agent that affects an immunological or physiological response in a subject (e.g., human). For example, an adjuvant might increase the presence of an antigen over time or to an area of interest like a tumor, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition (e.g., a pharmaceutical composition such as a solid dosage form of a pharmaceutical agent as described herein) to a subject. Examples of routes of administration include oral administration, rectal administration, topical administration, inhalation (nasal) or injection. Administration by injection includes intravenous (IV), intramuscular (IM), intratumoral (IT) and subcutaneous (SC) administration. A pharmaceutical composition described herein can be administered in any form by any effective route, including but not limited to intratumoral, oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In preferred embodiments, a pharmaceutical composition described herein is administered orally, rectally, intratumorally, topically, intravesically, by injection into or adjacent to a draining lymph node, intravenously, by inhalation or aerosol, or subcutaneously. In another preferred embodiment, a pharmaceutical composition described herein is administered orally, intratumorally, or intravenously. In another embodiment, a pharmaceutical composition described herein is administered orally.

As used herein, “anaerobic conditions” are conditions with reduced levels of oxygen compared to normal atmospheric conditions. For example, in some embodiments anaerobic conditions are conditions wherein the oxygen levels are partial pressure of oxygen (pO₂) no more than 8%. In some instances, anaerobic conditions are conditions wherein the pO₂ is no more than 2%. In some instances, anaerobic conditions are conditions wherein the pO₂ is no more than 0.5%. In certain embodiments, anaerobic conditions may be achieved by purging a bioreactor and/or a culture flask with a gas other than oxygen such as, for example, nitrogen and/or carbon dioxide (CO₂).

“Cancer” broadly refers to an uncontrolled, abnormal growth of a host's own cells leading to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s)” and “neoplasm(s)” are used herein interchangeably. As used herein, “cancer” refers to all types of cancer or neoplasm or malignant tumors including leukemias, carcinomas and sarcomas, whether new or recurring. Specific examples of cancers are: carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma. In some embodiments, the cancer comprises a solid tumor. In some embodiments, the cancer comprises a metastasis.

A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells. Environments of particular interest are the microenvironments where cancer cells reside or locate. In some instances, the microenvironment is a tumor microenvironment or a tumor draining lymph node. In other instances, the microenvironment is a pre-cancerous tissue site or the site of local administration of a composition or a site where the composition will accumulate after remote administration.

A “combination” of bacteria from two or more strains includes the physical co-existence of the bacteria, either in the same material or product or in physically connected products, as well as the temporal co-administration or co-localization of the bacteria from the two or more strains.

A “combination” of mEVs (such as smEVs and/or pmEVs) from two or more microbial (such as bacteria) strains includes the physical co-existence of the microbes from which the mEVs (such as smEVs and/or pmEVs) are obtained, either in the same material or product or in physically connected products, as well as the temporal co-administration or co-localization of the mEVs (such as smEVs and/or pmEVs) from the two or more strains.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size).

“Dysbiosis” refers to a state of the microbiota or microbiome of the gut or other body area, including; e.g., mucosal or skin surfaces (or any other microbiome niche) in which the normal diversity and/or function of the host gut microbiome ecological networks “microbiome”) are disrupted. A state of dysbiosis may result in a diseased state, or it may be unhealthy under only certain conditions or only if present for a prolonged period. Dysbiosis may be due to a variety of factors, including, environmental factors, infectious agents, host genotype, host diet and/or stress. A dysbiosis may result in: a change (e.g., increase or decrease) in the prevalence of one or more bacteria types (e.g., anaerobic), species and/or strains, change (e.g., increase or decrease) in diversity of the host microbiome population composition; a change (e.g., increase or reduction) of one or more populations of symbiont organisms resulting in a reduction or loss of one or more beneficial effects; overgrowth of one or more populations of pathogens (e.g., pathogenic bacteria); and/or the presence of, and/or overgrowth of, symbiotic organisms that cause disease only when certain conditions are present.

As used herein, “engineered bacteria” are any bacteria that have been genetically altered from their natural state by human activities, and the progeny of any such bacteria. Engineered bacteria include, for example, the products of targeted genetic modification, the products of random mutagenesis screens and the products of directed evolution.

The term “epitope” means a protein determinant capable of specific binding to an antibody or T cell receptor. Epitopes usually consist of chemically active surface groupings of molecules such as amino acids or sugar side chains. Certain epitopes can be defined by a particular sequence of amino acids to which an antibody is capable of binding.

The term “gene” is used broadly to refer to any nucleic acid associated with a biological function. The term “gene” applies to a specific genomic sequence, as well as to a cDNA or an mRNA encoded by that genomic sequence.

As used herein, “hemoglobin dependent bacteria” refers to bacteria for which growth rate is slowed and/or maximum cell density is reduced when cultured in growth media lacking hemoglobin, a hemoglobin derivative or spirulina when compared to the same growth media containing hemoglobin, a hemoglobin derivative or spirulina.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Mrtin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., psoriasis, atopic dermatitis, lupus, scleroderma, hemolytic anemia, vasculitis, type one diabetes, Grave's disease, rheumatoid arthritis, multiple sclerosis, Goodpasture's syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject's immune system to treat disease (e.g., immune disease, inflammatory disease, metabolic disease, cancer) and includes, for example, checkpoint inhibitors, cancer vaccines, cytokines, cell therapy, CAR-T cells, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold greater after treatment when compared to a pre-treatment state. Properties that may be increased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size).

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. immuno-adjuvants are a specific class of broader adjuvant or adjuvant therapy. Examples of STING agonists include, but are not limited to, 2′3′-cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLR1O and TLRI1. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The “internal transcribed spacer” or “ITS” is a piece of non-functional RNA located between structural ribosomal RNAs (rRNA) on a common precursor transcript often used for identification of eukaryotic species in particular fungi. The rRNA of fungi that forms the core of the ribosome is transcribed as a signal gene and consists of the 8S, 5.8S and 28S regions with ITS4 and 5 between the 8S and 5.8S and 5.8S and 28S regions, respectively. These two intercistronic segments between the 18S and 5.8S and 5.8S and 28S regions are removed by splicing and contain significant variation between species for barcoding purposes as previously described (Schoch et al Nuclear ribosomal internal transcribed spacer (ITS) region as a universal DNA barcode marker for Fungi. PNAS 109:6241-6246. 2012). 18S rDNA is traditionally used for phylogenetic reconstruction however the ITS can serve this function as it is generally highly conserved but contains hypervariable regions that harbor sufficient nucleotide diversity to differentiate genera and species of most fungus.

The term “isolated” or “enriched” encompasses a microbe (such as a bacterium), an mEV (such as an smEV and/or pmEV) or other entity or substance that has been (1) separated from at least some of the components with which it was associated when initially produced (whether in nature or in an experimental setting), and/or (2) produced, prepared, purified, and/or manufactured by the hand of man. Isolated microbes or mEVs may be separated from at least about 10%, about 20%, about 30%, about 40%, about 50%, about 60%, about 70%, about 80%, about 90%, or more of the other components with which they were initially associated. In some embodiments, isolated microbes or mEVs are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a microbe or mEV or other material that has been separated from at least some of the components with which it was associated either when initially produced or generated (e.g., whether in nature or in an experimental setting), or during any time after its initial production. A microbe or a microbial population or mEVs may be considered purified if it is isolated at or after production, such as from a material or environment containing the microbe or microbial population, and a purified microbe or microbial population or mEVs may contain other materials up to about 10%, about 20%, about 30%, about 40%, about 50%, about 60%, about 70%, about 80%, about 90%, or above about 90% and still be considered “isolated.” In some embodiments, purified microbes or microbial population or mEVs are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components (such as mEVs) thereof are generally purified from residual habitat products.

As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans).

The term “LPS mutant or lipopolysaccharide mutant” broadly refers to selected bacteria that comprises loss of LPS. Loss of LPS might be due to mutations or disruption to genes involved in lipid A biosynthesis, such as lpxA, lpxC, and lpxD. Bacteria comprising LPS mutants can be resistant to aminoglycosides and polymyxins (polymyxin B and colistin).

“Metabolite” as used herein refers to any and all molecular compounds, compositions, molecules, ions, co-factors, catalysts or nutrients used as substrates in any cellular or microbial metabolic reaction or resulting as product compounds, compositions, molecules, ions, co-factors, catalysts or nutrients from any cellular or microbial metabolic reaction.

“Microbe” refers to any natural or engineered organism characterized as a archaeaon, parasite, bacterium, fungus, microscopic alga, protozoan, and the stages of development or life cycle stages (e.g., vegetative, spore (including sporulation, dormancy, and germination), latent, biofilm) associated with the organism. Examples of gut microbes include: Actinomyces graevenitzii, Actinomyces odontolyticus, Akkermansia muciniphila, Bacteroides caccae, Bacteroides fragilis, Bacteroides putredinis, Bacteroides thetaiotaomicron, Bacteroides vultagus, Bifidobacterium adolescentis, Bifidobacterium bifidum, Bilophila wadsworthia, Blautia, Butyrivibrio, Campylobacter gracilis, Clostridia cluster III, Clostridia cluster IV, Clostridia cluster IX (Acidaminococcaceae group), Clostridia cluster XI, Clostridia cluster XIII (Peptostreptococcus group), Clostridia cluster XIV Clostridia cluster XV Collinsella aerofaciens, Coprococcus, Corynebacterium sunsvallense, Desulfomonas pigra, Dorea formicigenerans, Dorea longicatena, Escherichia coli, Eubacterium hadrum, Eubacterium rectale, Faecalibacteria prausnitzii, Gemella, Lactococcus, Lanchnospira, Mollicutes cluster XVI, Mollicutes cluster XVIII, Prevotella, Rothia mucilaginosa, Ruminococcus callidus, Ruminococcus gnavus, Ruminococcus torques, and Streptococcus.

“Microbial extracellular vesicles” (mEVs) can be obtained from microbes such as bacteria, archaea, fungi, microscopic algae, protozoans, and parasites. In some embodiments, the mEVs are obtained from bacteria. mEVs include secreted microbial extracellular vesicles (smEVs) and processed microbial extracellular vesicles (pmEVs). “Secreted microbial extracellular vesicles” (smEVs) are naturally-produced vesicles derived from microbes. smEVs are comprised of microbial lipids and/or microbial proteins and/or microbial nucleic acids and/or microbial carbohydrate moieties, and are isolated from culture supernatant. The natural production of these vesicles can be artificially enhanced (e.g., increased) or decreased through manipulation of the environment in which the bacterial cells are being cultured (e.g., by media or temperature alterations). Further, smEV compositions may be modified to reduce, increase, add, or remove microbial components or foreign substances to alter efficacy, immune stimulation, stability, immune stimulatory capacity, stability, organ targeting (e.g., lymph node), absorption (e.g., gastrointestinal), and/or yield (e.g., thereby altering the efficacy). As used herein, the term “purified smEV composition” or “smEV composition” refers to a preparation of smEVs that have been separated from at least one associated substance found in a source material (e.g., separated from at least one other microbial component) or any material associated with the smEVs in any process used to produce the preparation. It can also refer to a composition that has been significantly enriched for specific components. “Processed microbial extracellular vesicles” (pmEVs) are a non-naturally-occurring collection of microbial membrane components that have been purified from artificially lysed microbes (e.g., bacteria) (e.g., microbial membrane components that have been separated from other, intracellular microbial cell components), and which may comprise particles of a varied or a selected size range, depending on the method of purification. A pool of pmEVs is obtained by chemically disrupting (e.g., by lysozyme and/or lysostaphin) and/or physically disrupting (e.g., by mechanical force) microbial cells and separating the microbial membrane components from the intracellular components through centrifugation and/or ultracentrifugation, or other methods. The resulting pmEV mixture contains an enrichment of the microbial membranes and the components thereof (e.g., peripherally associated or integral membrane proteins, lipids, glycans, polysaccharides, carbohydrates, other polymers), such that there is an increased concentration of microbial membrane components, and a decreased concentration (e.g., dilution) of intracellular contents, relative to whole microbes. For gram-positive bacteria, pmEVs may include cell or cytoplasmic membranes. For gram-negative bacteria, a pmEV may include inner and outer membranes. pmEVs may be modified to increase purity, to adjust the size of particles in the composition, and/or modified to reduce, increase, add or remove, microbial components or foreign substances to alter efficacy, immune stimulation, stability, immune stimulatory capacity, stability, organ targeting (e.g., lymph node), absorption (e.g., gastrointestinal), and/or yield (e.g., thereby altering the efficacy). pmEVs can be modified by adding, removing, enriching for, or diluting specific components, including intracellular components from the same or other microbes. As used herein, the term “purified pmEV composition” or “pmEV composition” refers to a preparation of pmEVs that have been separated from at least one associated substance found in a source material (e.g., separated from at least one other microbial component) or any material associated with the pmEVs in any process used to produce the preparation. It can also refer to a composition that has been significantly enriched for specific components.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state (e.g., precancerous or cancerous state) or treatment conditions (e.g., antibiotic treatment, exposure to different microbes). In some aspects, the microbiome occurs at a mucosal surface. In some aspects, the microbiome is a gut microbiome. In some aspects, the microbiome is a tumor microbiome.

“Modified” in reference to a bacteria broadly refers to a bacteria that has undergone a change from its wild-type form. Bacterial modification can result from engineering bacteria. Examples of bacterial modifications include genetic modification, gene expression modification, phenotype modification, formulation modification, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium such that it increases or decreases virulence. Derivatives (such as mEVs) of modified bacteria may be considered as modified (e.g., modified mEVs).

An “oncobiome” as used herein comprises tumorigenic and/or cancer-associated microbiota, wherein the microbiota comprises one or more of a virus, a bacterium, a fungus, a protist, a parasite, or another microbe.

“Oncotrophic” or “oncophilic” microbes and bacteria are microbes that are highly associated or present in a cancer microenvironment. They may be preferentially selected for within the environment, preferentially grow in a cancer microenvironment or hone to a said environment.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide,” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, the term “preventing” a disease or condition in a subject refers to administering to the subject to a pharmaceutical treatment, e.g., the administration of one or more agents (e.g., pharmaceutical agent), such that onset of at least one symptom of the disease or condition is delayed or prevented.

As used herein, a substance is “pure” if it is substantially free of other components. The terms “purify,” “purifying” and “purified” refer to an mEV (such as an smEV and/or a pmEV) preparation or other material that has been separated from at least some of the components with which it was associated either when initially produced or generated (e.g., whether in nature or in an experimental setting), or during any time after its initial production. An mEV (such as an smEV and/or a pmEV) preparation or compositions may be considered purified if it is isolated at or after production, such as from one or more other bacterial components, and a purified microbe or microbial population may contain other materials up to about 10%, about 20%, about 30%, about 40%, about 50%, about 60%, about 70%, about 80%, about 90%, or above about 90% and still be considered “purified.” In some embodiments, purified mEVs (such as smEVs and/or pmEVs) are more than about 80%, about 85%, about 90%, about 91%, about 92%, about 93%, about 94%, about 95%, about 96%, about 97%, about 98%, about 99%, or more than about 99% pure. mEV (such as an smEV and/or a pmEV) compositions (or preparations) are, e.g., purified from residual habitat products.

As used herein, the term “purified mEV composition” or “mEV composition” refers to a preparation that includes mEVs (such as smEVs and/or pmEVs) that have been separated from at least one associated substance found in a source material (e.g., separated from at least one other bacterial component) or any material associated with the mEVs (such as smEVs and/or pmEVs) in any process used to produce the preparation. It also refers to a composition that has been significantly enriched or concentrated. In some embodiments, the mEVs (such as smEVs and/or pmEVs) are concentrated by 2 fold, 3-fold, 4-fold, 5-fold, 10-fold, 100-fold, 1000-fold, 10,000-fold or more than 10,000 fold.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about M or less, and binds to the predetermined antigen/binding partner with an affinity (as expressed by K_{D}) that is at least 10 fold less, at least 100 fold less or at least 1000 fold no more than its affinity for binding to a non-specific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The terms “subject” or “patient” refers to any mammal. A subject or a patient described as “in need thereof” refers to one in need of a treatment (or prevention) for a disease. Mammals (i.e., mammalian animals) include humans, laboratory animals (e.g., primates, rats, mice), livestock (e.g., cows, sheep, goats, pigs), and household pets (e.g., dogs, cats, rodents). The subject may be a human. The subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject may be healthy, or may be suffering from a cancer at any developmental stage, wherein any of the stages are either caused by or opportunistically supported of a cancer associated or causative pathogen, or may be at risk of developing a cancer, or transmitting to others a cancer associated or cancer causative pathogen. In some embodiments, a subject has lung cancer, bladder cancer, prostate cancer, plasmacytoma, colorectal cancer, rectal cancer, Merkel Cell carcinoma, salivary gland carcinoma, ovarian cancer, and/or melanoma. The subject may have a tumor. The subject may have a tumor that shows enhanced macropinocytosis with the underlying genomics of this process including Ras activation. In other embodiments, the subject has another cancer. In some embodiments, the subject has undergone a cancer therapy.

As used herein, a “systemic effect” in a subject treated with a pharmaceutical composition containing bacteria or mEVs (e.g., a pharmaceutical agent comprising bacteria or mEVs) of the instant invention means a physiological effect occurring at one or more sites outside the gastrointestinal tract. Systemic effect(s) can result from immune modulation (e.g., via an increase and/or a reduction of one or more immune cell types or subtypes (e.g., CD8+ T cells) and/or one or more cytokines). Such systemic effect(s) may be the result of the modulation by bacteria or mEVs of the instant invention on immune or other cells (such as epithelial cells) in the gastrointestinal tract which then, directly or indirectly, result in the alteration of activity (activation and/or deactivation) of one or more biochemical pathways outside the gastrointestinal tract. The systemic effect may include treating or preventing a disease or condition in a subject.

As used herein, the term “treating” a disease in a subject or “treating” a subject having or suspected of having a disease refers to administering to the subject to a pharmaceutical treatment, e.g., the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof.

As used herein, a value is “greater than” another value if it is higher by any amount (e.g., each of 100, 50, 20, 12, 11, 10.6, 10.1, 10.01, and 10.001 is at least 10). Similarly, as used herein, a value is “less than” another value if it is lower by any amount (e.g., each of 1, 2, 4, 6, 8, 9, 9.2, 9.4, 9.6, 9.8, 9.9, 9.99, 9.999 is no more than 10). In contrast, as used herein, a test value “is” an anchor value when the test value rounds to the anchor value (e.g., if “an ingredient mass is 10% of a total mass,” in which case 10% is the anchor value, the test values of 9.5, 9.6, 9.7, 9.8, 9.9, 10, 10.1, 10.2, 10.3, and 10.4 would also meet the “ingredient mass is 10% of the total mass” feature).

### Components of Spirulina

Spirulina and/or certain spirulina-derived components (e.g., soluble spirulina components) can be used in place of hemoglobin in growth media to facilitate the in vitro culturing of otherwise hemoglobin-dependent bacteria. Spirulina is a biomass of Arthrospira platensis and/or Arthrospira maxima cyanobacteria that has been consumed by humans for centuries in Mexico and some African countries. More recently, spirulina has been recognized as a rich source of proteins and many nutrients, and is therefore commonly consumed as a nutritional supplement. As spirulina is relatively inexpensive, vegetarian, kosher, and readily available at GMP-grade, it is an attractive alternative to hemoglobin in bacterial cell culture applications.

As demonstrated herein, culturing bacteria in growth media comprising spirulina results in pharmaceutical agents and/or pharmaceutical compositions and/or solid dosage forms comprising said bacteria or agents therefrom (e.g., mEVs), and comprising spirulina or components thereof. The present disclosure provides such agents and compositions and use thereof, as well as methods to test said agents and compositions to detect the presence of spirulina or components thereof.

Various components of spirulina are present and can be detected in the pharmaceutical agents and/or pharmaceutical compositions and/or solid dosage forms described herein, including spirulina nucleic acids, spirulina proteins and/or spirulina small molecules, such as pigments.

In certain embodiments, the pharmaceutical agents and/or pharmaceutical compositions and/or solid dosage forms of the present disclosure comprise a spirulina nucleic acid.

In some embodiments, the component of spirulina is a spirulina nucleic acid. As used herein, the spirulina nucleic acid is intended to include DNA (e.g., genomic DNA, cDNA) and RNA (e.g., mRNA, tRNA, rRNA, coding RNA, non-coding RNA, small RNA, etc.). The nucleic acid molecule can be single-stranded or double-stranded. In certain embodiments, the spirulina nucleic acid is spirulina genomic DNA. The spirulina nucleic acid can comprise a coding sequence (e.g., a sequence encoding a spirulina protein). In some embodiments, the spirulina nucleic acid comprises only a portion of a sequence encoding a spirulina protein. In some embodiments, the spirulina nucleic acid comprises a non-coding sequence.

In certain embodiments, the nucleic acid is spirulina DNA. Exemplary spirulina genomic nucleic acid sequences can be derived from NCBI Reference Sequence: NZ_AFXD00000000.1 Arthrospira platensis C1, whole genome shotgun sequencing project (world wide web at ncbi.nlm.nih.gov/nuccore/NZ_AFXD00000000.1). The genome structure of A. platensis is estimated to be a single, circular chromosome of 6.8 Mb, based on optical mapping. Annotation of this 6.7 Mb sequence yielded 6630 protein-coding genes (see Fujisawa et al. (2010) DNA Res. 17:85-103).

In some embodiments, the spirulina DNA comprises a sequence encoding a spirulina protein. In some embodiments, the spirulina DNA comprises a sequence encoding C-phycocyanin alpha subunit (cpcA), phycocyanin alpha subunit phycocyanobilin lyase (cpcE), phycocyanin alpha subunit phycocyanobilin lyase (cpcF), or chlorophyll a synthase (ChIG). In some embodiments, the spirulina DNA comprises a sequence encoding C-phycocyanin alpha subunit (cpcA) or chlorophyll a synthase (ChIG).

In certain embodiments, the pharmaceutical agents and/or pharmaceutical compositions and/or solid dosage forms of the present disclosure comprise a spirulina protein. In some embodiments, the spirulina protein is phycocyanin. Phycocyanin is a pigment-protein complex from the light-harvesting phycobiliprotein family. It is an accessory pigment to chlorophyll that is found in spirulina but not in hemoglobin-dependent bacteria. In some embodiments, the phycocyanin is C-phycocyanin alpha subunit (cpcA), phycocyanin alpha subunit phycocyanobilin lyase (cpcE), phycocyanin alpha subunit phycocyanobilin lyase (cpcF), or chlorophyll a synthase (ChIG). In some embodiments, the phycocyanin is C-phycocyanin alpha subunit (cpcA) or chlorophyll a synthase (ChIG).

In certain embodiments, the pharmaceutical agents and/or pharmaceutical compositions and/or solid dosage forms of the present disclosure comprise a spirulina small molecule. In some embodiments, the spirulina small molecule is a spirulina pigment. In some embodiments, the spirulina pigment is a chlorophyllin or beta carotene.

Exemplary nucleic sequences of components of spirulina are presented below in Table A.

TABLE A
Sequences of Exemplary Components of Spirulina

SEQ ID NO: 1 Arthrospira platensis chlorophyll synthase ChlG amino acid sequence
(NCBI Reference Sequence WP_006619293.1)

| | |
|---|---|
| 1 | msdsptpets seaipnsgsk trqllgmkga apgetsiwki rlqlmkpitw ipliwgvvcg |
| 61 | aassggyswt ledilkaaac mllsgplmag ytqtlndfyd rdldainepy rpipsgaisi |
| 121 | pqvvsqilil lgagiglayi ldiwaghefp mvtvlcigga fvsyiysapp lklkkngwlg |
| 181 | nyalgasyia lpwwaghalf gelnptivvl tlfyslaglg iaivndfksv egdrqlglqs |
| 241 | lpvmfgvtta awicvlmidi fqagvalyli siqqnlyati llllvipqit fqdmyflrnp |
| 301 | lendvkyqas aqpflvlgml vvglalghav |

SEQ ID NO: 2 Arthrospira platensis chlorophyll synthase (ChlG) cDNA sequence (NCBI
Sequence: NC_016640.1)

| | |
|---|---|
| 1 | atgtctgatt ccccaacccc cgaaacctct tcagaagcca tccccaactc cgggtcaaaa |
| 61 | acccgccagt tactagggat gaaaggagcc gcccccggag aaacctccat ctggaaaatt |
| 121 | cgcctgcaac tgatgaaacc aatcacctgg attcccctaa tttggggtgt agtctgtggt |
| 181 | gcggcttcct ctggaggcta tagctggaca ctagaagata tectcaaagc agccgcctgt |
| 241 | atgctgctgt cgggtccttt aatggcaggg tacacccaaa ccctcaacga cttctacgat |
| 301 | cgcgacctag atgccattaa cgagccctat cgccccattc cctccggtgc aatttccatc |
| 361 | ccccaagtcg tatcccaaat tctgattctc ctaggtgcgg gtattggtct tgcctatatc |
| 421 | ctggatattt gggcaggtca tgaattcccc atggtcacag ttctctgtat tggcggcgct |
| 481 | ttcgtttcct atatctactc tgcgcctccc ctaaaactca agaaaaacgg ctggttaggc |
| 541 | aactatgccc tcggagctag ttatattgcc cttccctggt gggcgggtca tgccctgttt |
| 601 | ggagaactca accccactat tgtcgtactc accctattct acagcttggc ggggctaggt |
| 661 | attgccattg tcaatgactt taagagtgtc gaaggcgatc gccaacttgg cttacaatcc |
| 721 | ctccccgtta tgtttggcgt aaccaccgcc gcctggatct gtgtattaat gatcgacatt |
| 781 | tttcaagccg gagttgccct atacttgatc agcattcagc aaaacctgta tgccaccatc |
| 841 | cttttattac tcgtgattcc acaaattacc ttccaagata tgtattttct tcgcaatccc |
| 901 | ctcgaaaatg atgttaaata tcaggcaagc gctcaacctt tcctggtgtt aggaatgtta |
| 961 | gtcgtgggtt tagccctagg tcatgcagtt tag |

SEQ ID NO: 3 Arthrospira platensis C-phycocyanin alpha subunit (cpcA) amino acid
sequence (NCBI Reference Sequence: YP_005068163)

| | |
|---|---|
| 1 | mktplteavs iadsqgrfls steiqvafgr frqakaglea akaltskads lisgaaqavy |
| 61 | nkfpyttqmq gpnyaadqrg kdkcardigy ylrmvtycli aggtgpmdey liagideinr |
| 121 | tfelspswyi ealkyikanh glsgdaavea nsyldyaina ls |

SEQ ID NO: 4 Arthrospira platensis C-phycocyanin alpha subunit (cpcA) nucleic acid
sequence (NCBI Reference Sequence: NC_016640.1)

| | |
|---|---|
| 1 | ctagctcagg gcgttgatcg cgtagtcgag gtaggagtta gcttcaacag cagcgtcacc |
| 61 | agacaaaccg tggttagctt tgatgtattt cagggcttca atgtaccagc ttggagaaag |
| 121 | ctcgaaagtc cggttgattt catcaatacc ggcaatcagg tactcatcca tggggccagt |
| 181 | tccaccagca atcaggcaat aagttaccat ccgcaggtag tagcctatgt cacgagcaca |
| 241 | tttgtcctta ccgcgttggt ctgccgcgta gttaggtccc tgcatttggg tggtgtaggg |
| 301 | gaacttgttg tacactgctt gggcagcacc actgatcaga ctatcagctt tagaggtcaa |
| 361 | agctttagca gcttccagac cagctttggc ttgacgaaaa cggccaaaag ctacttggat |
| 421 | ttcggtgctg cttaggaaac gaccttggga atcagcgata gaaactgctt cggttagggg |
| 481 | ggttttcat |

SEQ ID NO: 5 Arthrospira platensis phycocyanin alpha subunit phycocyanobilin lyase
(cpcE) amino acid sequence (NCBI Reference Sequence: YP_005068159)

| | |
|---|---|
| 1 | mqdsesktpg asplvgadna pltggeaegg dawtvegaia alnhqepnrr yyaawwlgrf |
| 61 | rvdepiavda liralddesd rtadggyplr rnaaralgkl garravtplv kcldcpdfyv |
| 121 | reaaaqaleg lgdsscasal rgllvggved tepiagkphl kqpydaviea igtlgateal |
| 181 | adiepfinhp iariqyaalr amyqltgdrv ygdrlivalg gedlqlrrsa lmdlgaigyl |
| 241 | eaaepiantl aenslklial qgilehhlsq igdkclseea vkimklmdsl l |

SEQ ID NO: 6 Arthrospira platensis phycocyanin alpha subunit phycocyanobilin lyase
(cpcE) nucleic acid sequence (NCBI Reference Sequence: NC_016640.1)

| | |
|---|---|
| 1 | atgcaggatt ctgaatcaaa aaccccaggt gcatcccccc tagtgggcgc agataatgcg |
| 61 | cctttaactg ggggagaagc tgaagggggt gatgcttgga cagtagaaca ggcgatcgcc |
| 121 | gccctaaacc atcaagaacc aaatcgtcgc tattatgcgg cttggtggtt ggggcgtttt |
| 181 | cgtgttgatg aaccgatcgc agttgatgcg ttaattaggg ctttggatga tgagtccgat |
| 241 | cgcacggccg acgggggcta ccctttacgg agaaatgcgg ctcgggcttt gggtaaattg |
| 301 | ggcgcgcggc gcgcggtgac tcccctagtc aaatgcttag attgcccaga tttttatgta |
| 361 | cgagaggcgg cggctcaggc tttggaaggt ctgggagact ctagttgtgc ttcggctttg |
| 421 | cgtgggttat tggtgggtgg agtagaagat actgaaccga tagcgggtaa accccattta |
| 481 | aaacagcctt atgatgcggt aattgaagcc ataggaaccc tgggtgcaac tgaggcgtta |
| 541 | gctgatattg aaccttttat aaaccatccg atcgctagaa tacaatatgc ggctttgagg |
| 601 | gctatgtatc agcttacggg tgatcgggtt tatggcgatc gcttaattgt ggctttgggg |
| 661 | ggggaagatt tacaattgcg acgcagtgct ttaatggatt taggggcgat cggatacctt |
| 721 | gaggcggcgg aacctatcgc caatacccta gotgaaaata gcctgaaact aattgcttta |
| 781 | cagggaattc tggaacatca tctcagtcaa attggcgaca aatgcctatc tgaagaggca |
| 841 | gttaaaatca tgaagctgat ggattctcta ttatga |

SEQ ID NO: 7 Arthrospira platensis phycocyanin alpha subunit phycocyanobilin lyase
(cpcF) amino acid sequence (NCBI Reference Sequence: YP_005068158)

| | |
|---|---|
| 1 | mtrveelila veqadsagkl igavsqlaat gsvdavpmli rvlgynnpga avaaveglia |
| 61 | igkpavvgll erldgynyga rawavralsg igdprgldvl leaagsdfal svrraaargl |
| 121 | gaihwedmss evvseakert lktllhtsed tewivryatv vgleglaase vdkpnwftqv |
| 181 | aerlegmvnn detpavsara slarvnlgis |

SEQ ID NO: 8 Arthrospira platensis phycocyanin alpha subunit phycocyanobilin lyase
nucleic acid sequence Reference Sequence: NC_016640.1)

| | |
|---|---|
| 1 | atgacaaggg ttgaggaact aattttagcg gttgaacagg cggactctgc gggaaaactg |
| 61 | ataggggcgg tgtcacaatt ggcggcgacg ggttctgtgg atgcagtacc aatgttaatt |
| 121 | cgtgttttgg gttacaacaa ccctggggcg gcggtggcgg cggttgaggg attaatagcc |
| 181 | attggtaaac ctgctgtggt ggggctacta gagaggttag atggttataa ctatggtgct |
| 241 | agggcttggg cggtgcgggc tttgtctggt attggtgatc ctaggggttt ggatgttttg |
| 301 | ctggaggcgg cgggtagtga ttttgctctg agtgtgcgtc gtgcggcggc gcggggtttg |
| 361 | ggagcaattc actgggagga tatgtcatca gaagtggtgt ctgaggctaa ggaaagaacc |
| 421 | ctaaaaactc tgctgcatac ttctgaagat acggaatgga tcgttcgtta tgcgacggtt |
| 481 | gtgggtttgg aaggtttggc ggcttctgag gtggataagc ccaattggtt tacacaggtt |
| 541 | gctgaaaggt tggaggggat ggttaataat gatgaaactc cggcggtttc tgctagggct |
| 601 | agtctggcga gggtaaactt agggattagt taa |

*Included in Table A are RNA nucleic acid molecules (e.g., thymidine replaced with uridines), as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table A, or a portion thereof.

### Detection of Components of Spirulina

In certain aspects, the methods and compositions provided herein relate to methods of testing a pharmaceutical agent or pharmaceutical composition or solid dosage form to detect (e.g., assay for) the presence of a component of spirulina in the pharmaceutical agent or pharmaceutical composition or solid dosage form. Any suitable methods described herein or those known in the art can be used to detect a component of spirulina. In certain embodiments, samples for detection can be readily prepared by dissolving the pharmaceutical agent or pharmaceutical composition or solid dosage form in an appropriate buffer/medium prior to testing.

In some embodiments, the component of spirulina comprises a spirulina nucleic acid. In some embodiments, the spirulina nucleic acid is spirulina DNA. In some embodiments, the spirulina DNA comprises a sequence encoding C-phycocyanin alpha subunit (cpcA) or chlorophyll a synthase (ChIG). In certain embodiments, the spirulina nucleic acid (e.g., DNA) is detected using a nucleic acid amplification assay, a sequencing assay, and/or a microarray assay. In preferred embodiments, the presence of a component of spirulina (e.g., nucleic acid) is detected using a polymerase chain reaction (PCR) assay, such as quantitative polymerase chain reaction (qPCR) assay or digital PCR.

In some embodiments, the component of spirulina is a spirulina protein. In some embodiments, the spirulina protein is phycocyanin. In some embodiments, the component of spirulina comprises a spirulina small molecule (e.g., pigment). In some embodiments, the spirulina pigment is spirulina is chlorophyllin or beta carotene. In preferred embodiments, the spirulina protein or spirulina small molecule (e.g., pigment) is detected by high performance liquid chromatography (HPLC) or ultra performance liquid chromatography (UPLC). In some embodiments, the spirulina protein or spirulina small molecule (e.g., pigment) is detected by mass spectrometry (e.g., tandem mass spectrometry, MALDI-TOF).

### Nucleic Acid Detection

In certain aspects, the provided herein are methods related to the detection of spirulina nucleic acids.

In some embodiments, the spirulina nucleic acid may be isolated, however, it will be appreciated by those skilled in the art that many detection assays (e.g., PCR, e.g., qPCR or digital PCR) can be performed to detect the nucleic acid in a sample without isolating the nucleic acid. A nucleic acid can be isolated using standard molecular biology techniques and the sequence information in the public database records, e.g., NCBI, Uniprot, etc. Using all or a portion of such nucleic acid sequences, nucleic acid molecules of the present invention can be isolated using standard hybridization and cloning techniques (e.g., as described in Green and Sambrook, ed., Molecular Cloning: A Laboratory Manual, Fourth ed., Cold Spring Harbor Laboratory Press, Cold Spring Harbor, N Y, 2014).

In other embodiments, amplification-based assays can be used to determine the presence of amount of the spirulina DNA. In such amplification-based assays, the nucleic acid sequences act as a template in an amplification reaction (e.g., Polymerase Chain Reaction (PCR)). In a quantitative amplification, the amount of amplification product will be proportional to the amount of template in the original sample (see e.g., Kralik and Ricchi (2017) Front. Microbiol. 8:108). Comparison to appropriate controls, e.g., amplification of a bacterial nucleic acid sequence, provides a measure of the copy number or amount of the spirulina nucleic acid.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, digital PCR, and linker adapter PCR, etc.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

For determining the presence or amount of spirulina RNA by amplification methods, the RNA molecules can first be converted to cDNA by a reverse transcriptase (primer-specific cDNA synthesis), prior to performing the amplification methods. Various amplification and detection methods can be used. For example, it is within the scope of the present invention to reverse transcribe RNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

In some embodiments, the presence of spirulina nucleic acids can be detected in a pharmaceutical agent and/or pharmaceutical composition and/or solid dosage form provided herein by sequencing nucleic acids present in the pharmaceutical agent and/or pharmaceutical composition and/or drug product. In some embodiments, the sequencing assay used is a next generation sequencing (NGS) assay. Nucleic acid sequencing processes include, but are not limited to chain termination sequencing, sequencing by ligation, sequencing by synthesis, pyrosequencing, ion semiconductor sequencing, single-molecule real-time sequencing, and/or 454 sequencing. In some embodiments, the NGS Modality is any of the following: SwabSeq, 1 Amplicon, 384 well plate, 96 Nextera barcode set, UDI's, NextSeq; SwabSeq—1 Amplicon, 384 well plate, 384 Truseq UDI barcode set, using NextSeq; or SwabSeq—1 Amplicon, 384 well plate, 4000 UDI Truseq barcode set, NovaSeq. SwabSeq—Multiplex, 384 well plate, CDI barcode set, NovaSeq.

Many techniques are known in the state of the art for determining absolute and relative levels of a nucleic acid in a sample. In some embodiments, the presence and amount of spirulina nucleic acid can be detected on a DNA array, chip or a microarray. Labeled nucleic acids of a test sample (those present in the pharmaceutical agent or pharmaceutical composition or solid dosage form) obtained from a subject may be hybridized to a solid surface comprising the spirulina nucleic acid (e.g., DNA or RNA). Positive hybridization signal is obtained with the sample containing the spirulina nucleic acid. Methods of preparing DNA arrays and their use are well-known in the art (see, e.g., U.S. Pat. Nos: 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety).

Methods of detecting a spirulina nucleic acid include, but are not limited to, hybridization-based assays. Hybridization-based assays include traditional “direct probe” methods, such as Southern blots or Dot blots. The methods can be used in a wide variety of formats including, but not limited to, substrate (e.g. membrane or glass) bound methods or array-based approaches.

In some embodiments, detecting a spirulina DNA in a sample involves a Southern Blot. In a Southern Blot, the genomic DNA or fragments thereof (typically separated on an electrophoretic gel) is hybridized to a probe specific for the target region. Comparison of the intensity of the hybridization signal from the probe for the spirulina DNA with control probe signal from analysis of bacterial DNA provides an estimate of the amount of the spirulina nucleic acid. To increase the specificity of the assay, a probe hybridizes the spirulina DNA under stringent conditions. As used herein, the term “hybridizes under stringent conditions” is intended to describe conditions for hybridization and washing under which nucleotide sequences at least 60% (65%, 70%, 75%, 80%, 85%, 90%, 95%, 96%, 97%, 98%, 99%, or 100%; preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

Alternatively, a Northern blot may be utilized for evaluating the presence or amount of RNA in the pharmaceutical composition or solid dosage form of the present disclosure. In a Northern blot, RNA is hybridized to a probe specific for the spirulina RNA. Comparison of the intensity of the hybridization signal from the probe for the spirulina RNA with control probe signal from analysis of bacterial RNA provides an estimate of the relative amount of the spirulina RNA. A simpler version of the Southern blot and the Northern blot may be performed using a dot blot format, in which the sample comprising the spirulina DNA is simply spotted (without electrophoretic separation) on a platform and the hybridization method is carried out.

### Protein Detection

In certain aspects, the methods and compositions provided herein relate to the detection of spirulina proteins. The presence or amount of a spirulina protein in pharmaceutical agents and/or pharmaceutical compositions or solid dosage forms described herein can be detected by various methods known in the art. Exemplary methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Ten, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired protein standard (e.g., a known spirulina protein, the presence of which is being tested in a pharmaceutical agent and/or pharmaceutical composition or solid dosage form) is labeled (with a radioisotope such as ¹²⁵I or ³⁵S, or an assayable enzyme, such as horseradish peroxidase or alkaline phosphatase), and, together with the unlabelled sample (e.g., the spirulina protein present in the pharmaceutical agent and/or pharmaceutical composition or solid dosage form), brought into contact with the corresponding antibody, whereon a second antibody is used to bind the first, and radioactivity or the immobilized enzyme assayed (competitive assay). Alternatively, the spirulina protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

Enzymatic and radiolabeling of spirulina proteins and/or the antibodies may be affected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be affected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect a spirulina protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-spirulina protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Antibodies that may be used to detect a spirulina protein include any antibody, whether natural or synthetic, full length or a fragment thereof, monoclonal or polyclonal, that binds sufficiently strongly and specifically to the protein to be detected. An antibody may have a K_{d} of at most about 10⁻⁶ M, 10⁻⁷ M, 10⁻⁸ M, 10⁻⁹ M, 10⁻¹⁰ M, 10⁻¹¹M, or 10⁻¹² M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the target spirulina protein relative to other proteins, such as related proteins. Antibodies may be prepared according to methods known in the art.

In some embodiments, agents that specifically bind to a spirulina protein other than antibodies are used, such as peptides or small molecules. Peptides or small molecules that specifically bind to a biomarker protein can be identified by any means known in the art. For example, specific peptide binders of a target spirulina protein can be screened for using peptide phage display libraries.

A spirulina protein or fragment thereof may also be detected using mass spectrometry and/or HPLC and/or UPLC as for detection of a small molecule as described below.

### Small Molecule Detection

In certain embodiments, the methods and compositions provided herein relate to the detection of spirulina small molecules.

In certain embodiments, a chromatography method is used to detect a spirulina small molecule (or protein). Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), ultra performance liquid chromatography (UPLC), etc. In certain embodiments, the small molecule detected using a chromatography method is a pigment. In certain embodiments, a spirulina protein is detected using a chromatography method.

Any one or combination of the methods described herein can be used to detect (and quantify) the amount of at least one component of spirulina present in the pharmaceutical compositions or solid dosage form provided herein. In preferred embodiments, the chromatography is HPLC or UPLC. These methods provide sensitivity that allows separation and detection of a trace amount of at least one component of spirulina present in the pharmaceutical agent or pharmaceutical composition or the solid dosage form.

Small molecules, nucleic acids, or proteins or fragments thereof of spirulina can be detected and quantified using mass spectrometry with or without separation techniques. Mass spectrometry (MS) is an analytical technique that measures the mass-to-charge ratio of ions. The results are typically presented as a mass spectrum, a plot of intensity as a function of the mass-to-charge ratio. Mass spectrometry is used in many different fields and is applied to pure samples as well as complex mixtures.

A mass spectrum is a plot of the ion signal as a function of the mass-to-charge ratio. These spectra are used to determine the elemental or isotopic signature of a sample, the masses of particles and of molecules, and to elucidate the chemical identity or structure of molecules and other chemical compounds.

Various mass spectrometry-based methods can be utilized to detect the small molecules, nucleic acids, or proteins or fragments thereof of spirulina including, but are not limited to, tandem mass spectrometry (MS/MS), MALDI-TOF (a combination of a matrix-assisted laser desorption/ionization source with a time-of-flight mass analyzer), inductively coupled plasma-mass spectrometry (ICP-MS), accelerator mass spectrometry (AMS), thermal ionization-mass spectrometry (TIMS), isotope ratio mass spectrometry (IRMS), and spark source mass spectrometry (SSMS).

A tandem mass spectrometer is one capable of multiple rounds of mass spectrometry, usually separated by some form of molecule fragmentation. For example, one mass analyzer can isolate one peptide from many entering a mass spectrometer. A second mass analyzer then stabilizes the peptide ions while they collide with a gas, causing them to fragment by collision-induced dissociation (CID). A third mass analyzer then sorts the fragments produced from the peptides. Tandem MS can also be done in a single mass analyzer over time, as in a quadrupole ion trap. There are various methods for fragmenting molecules for tandem MS, including collision-induced dissociation (CID), electron capture dissociation (ECD), electron transfer dissociation (ETD), infrared multiphoton dissociation (IRMPD), blackbody infrared radiative dissociation (BIRD), electron-detachment dissociation (EDD) and surface-induced dissociation (SID). An important application using tandem mass spectrometry is in protein identification.

Mass spectrometry-based detection of spirulina small molecules, nucleic acids, or proteins or fragments thereof, can be enhanced by coupling it with chromatographic and/or other separation techniques. Separation may include any procedure known in the art, such as capillary electrophoresis (e.g., in capillary or on-chip) or chromatography (e.g., in capillary, column or on a chip, liquid chromatography, gas chromatography). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

### Bacteria

The pharmaceutical agents and/or pharmaceutical compositions and/or solid dosage forms disclosed herein can comprise bacteria and/or microbial extracellular vesicles (mEVs) (such as smEVs and/or pmEVs). For example, the pharmaceutical compositions and/or solid dosage forms disclosed herein can comprise a powder (e.g., pharmaceutical agent) comprising bacteria and/or microbial extracellular vesicles (mEVs) (such as smEVs and/or pmEVs). Within the pharmaceutical agents and/or pharmaceutical compositions and/or solid dosage forms that contain bacteria and mEVs, the mEVs can be from the same bacterial origin (e.g., same strain) as the bacteria of the pharmaceutical agent. The pharmaceutical agent can contain bacteria and/or mEVs from one or more strains.

In some embodiments, the bacteria of the pharmaceutical agent or from which the mEVs of the pharmaceutical agent are obtained are hemoglobin-dependent bacteria. In some embodiments, the bacteria are hemoglobin-dependent bacteria. In some embodiments, the mEVs are from hemoglobin-dependent bacteria. As used herein, “hemoglobin-dependent bacteria” refers to bacteria for which growth rate is slowed and/or maximum cell density is reduced when cultured in growth media lacking hemoglobin, a hemoglobin derivative or spirulina when compared to the same growth media containing hemoglobin, a hemoglobin derivative or spirulina.

In some embodiments, the hemoglobin-dependent bacteria are from bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptomphilus, Peptostreptococcus, Porphyromonas, Prevotella, Propionibacterium, Rarimicrobium, Shuttleworthia, Turicibacter, or Veillonella.

In some embodiments, the hemoglobin-dependent bacteria are of the genus Fournierella. In some embodiments, the hemoglobin-dependent bacteria are Fournierella Strain A.

In some embodiments, the hemoglobin-dependent Fournierella strain is Fournierella Strain B (ATCC Deposit Number PTA-126696). In some embodiments, the hemoglobin-dependent Fournierella strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Fournierella Strain B (PTA-126696).

In some embodiments, the hemoglobin-dependent bacteria are of the genus Parabacteroides. In some embodiments, the hemoglobin-dependent bacteria are Parabacteroides Strain A. In some embodiments, the hemoglobin-dependent bacteria are Parabacteroides Strain B.

In some embodiments, the hemoglobin-dependent bacteria are of the genus Faecalibacterium. In some embodiments, the hemoglobin-dependent bacteria are Faecalibacterium Strain A.

In some embodiments, the hemoglobin-dependent bacteria are of the genus Bacteroides. In some embodiments, the hemoglobin-dependent bacteria are Bacteroides Strain A.

In some embodiments, the hemoglobin-dependent bacteria are of the genus Allistipes. In some embodiments, the hemoglobin-dependent bacteria are Allistipes Strain A.

In some embodiments, the hemoglobin-dependent bacteria are of the genus Prevotella. In some embodiments, the hemoglobin-dependent bacteria are of the species Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella melanogenica, Prevotella intermedia, Prevotella maculosa, Prevotella marshii, Prevotella melaninogenica, Prevotella micans, Prevotella multiformis, Prevotella nigrescens, Prevotella oxalis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis. In some embodiments, the hemoglobin-dependent bacteria are of the species Prevotella histicola.

In some embodiments, the hemoglobin-dependent bacteria are Alistipes indistinctus, Alistipes shahii, Alistipes timonensis, Bacillus coagulans, Bacteroides acidifaciens, Bacteroides cellulosilyticus, Bacteroides eggerthii, Bacteroides intestinalis, Bacteroides uniformis, Collinsella aerofaciens, Cloacibacillus evryensis, Clostridium cadaveris, Clostridium cocleatum, Cutibacterium acnes, Eisenbergiella sp., Erysipelotrichaceae sp., Eubacterium hallii/Anaerobutyricum halii, Eubacterium infirmum, Megasphaera micronuciformis, Parabacteroides distasonis, Peptoniphilus lacrimalis, Rarimicrobium hominis, Shuttleworthia satelles, or Turicibacter sanguinis.

In some embodiments, the hemoglobin-dependent Prevotella strain is Prevotella Strain B 50329 (NRRL accession number B 50329). In some embodiments, the hemoglobin-dependent Prevotella strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

In some embodiments, the hemoglobin-dependent Prevotella strain is Prevotella Strain C (ATCC Deposit Number PTA-126140). In some embodiments, the hemoglobin-dependent Prevotella strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain C (PTA-126140).

In some embodiments, the hemoglobin-dependent Prevotella strain is a strain of Prevotella bacteria comprising one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) proteins listed in Table 1 and/or one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) genes encoding proteins listed in Table 1. In some embodiments, the hemoglobin-dependent Prevotella strain comprises all of the proteins listed in Table 1 and/or all of the genes encoding the proteins listed in Table 1.

TABLE 1
Exemplary Prevotella proteins

| Seq. | | Uniprot | |
|---|---|---|---|
| ID. No. | Name | ID | Amino Acid Sequence |
| 9 | Cluster: | G6ADE1 | MNLKTFTKTVLCFALFAVSAITAKAADHLAIVGEAV |
| | Uncharacterized | | WGGWDLVKATAMVKSPNNPDVFMATVHLNAGKGF |
| | protein | | KFLTEREWGKLEYRSGASDVVLKSGIRYKLYASIGAS |
| | | | EDGKFKVSESANYEIICDLARKTVEVKKVAYQAKEIR |
| | | | YAALWMIGDATAGDWDYNNGVLLSQDSGNPTCYTA |
| | | | TVELKEGEFKFTTNKQWGYDHSVYIFRDVNDQNKIVF |
| | | | GGEDNKWRITEDGMYNVTVDVPTKTISIKQIDDPAGH |
| | | | KPQFGNDVILVGDATIAGWNLDNAIYLEHTGQAGRVF |
| | | | KTTTYLEAGKGFKFLSMLSYDDIDYRPANNTVLNPGV |
| | | | PGTFVPSLPSSTDTKFSVERSGNYDIVCNMNNRTVVV |
| | | | TLSENQVLVNYPALWLIGSATSAGWNPGKAVELKRS |
| | | | EADPAVYTARVQLKKGEFKILTSKNVGFDQPTYYRDS |
| | | | TNEHRIVFGVDGDEVAKKDCKWTLSENAEGTYDVTV |
| | | | DIEAMTIFCDKVNMDEPSVESTDKELILIGDATYSAWD |
| | | | LPKSIVMTPVGPTTFKAVTHLEAGKEFKFLTELAWKR |
| | | | YEYRAESLRKELQEGSMSMLVPYRYTNDKDDKDHDF |
| | | | KFVVKESGNYEIVCDLYIPALIIRKVRYQDTPVTYSSL |
| | | | WIVGSATPGGWTIERGIKMTQDENYPTKFTAKANLVP |
| | | | GELKFATNKFADFTQDFFFRGKDDYTAVLGGNDNKW |
| | | | NITEAGTYSVTIDVASKRVTITKPARNAPTGISTVDSSD |
| | | | EAPAEYFTLNGIKVTTPSSGIYIKRQGGRTTKVVMK |
| 10 | Nicotinamide_ | P24520 | MDTYQILDIIGCIVGLIYIYQEYKASIWLWMTGIIMPVI |
| | riboside_transporter_ | | YMFVYYEAGLYADFGMQIYYTLAAIYGYLYWKLGK |
| | PnuC | | KKGTEDKEIPITHFPRRYIIPAIIVFFVLWIALYYILICFT |
| | | | NSTVPVLDSFGNALSFIGLWALAKKYLEQWWIWIVV |
| | | | DAELSALYIYKGIPFTAMLYALYTVIAVAGYFKWRRY |
| | | | IKQQK |
| 11 | Pectate_trisaccharide- | Q8GCB2 | MRVRLYKNILLFLFLWVNTLACVSADTSRTVESQPIE |
| | lyase | | NGLIITESKGWLETIYAKWKPVAEADGYYVYVKGGQ |
| | | | YADYSKVDSELIRVYNGYVRVDIPGLKAGTYSLKIVA |
| | | | VKGGKETQSSEVTGLKVLNYVREGFAHKNYSGVGAY |
| | | | NDDGTLKSGAVVIYVNKDNAKTVSAHLGKTTFIGLQ |
| | | | AILNAYQKGNITTPLSVRILGLLRNGDTDTFGSSTEGIQ |
| | | | IKGKQADSEMNITIEGIGEDASIYGFGFLVRNAKSVEF |
| | | | RNLGIMRAMDDGVSLDTNNSNIWIHHMDLFYGKASG |
| | | | GDHIKGDGSIDVKTDSKYVTIDNCHFWDTGKTSMCG |
| | | | MKKETGPNYITYHHNWFDHSDSRHARVRTMSVHLW |
| | | | NNYYDGCAKYGIGATMGCSVFSENNYFRATKNPILIS |
| | | | KQGSDAKGTGKFSGEPGGMVKEYGSLFTEKGAESTY |
| | | | TPISYADNNSSFDFYHAISRNEKVPASVKTLNGGNIYN |
| | | | NFDTDAALMYSYTPDATALVPSQVTGFYGAGRLNHG |
| | | | SLQFKFNNAVEDTNSTPIPALEALIDAYSGK |
| 12 | Glycosyltransferase | Q9AET5 | MKYNIAYCIEGFYNHGGMERILSVCANLLSDIYSITIIV |
| | Gtf1 | | ANQRGREHAYNLAQNVNVVDLGVSCKNYKEEYKKS |
| | | | LTRYLQDHQFSVVISLAGLELFFLPQIKDGSKKVMWF |
| | | | HFAFDVSKMFLSERFHGWKLNLLYYIHTIRRIYFAKKF |
| | | | DTIVVLSKSDCDSWSRFCNNVKYIYNPITIDRKVISNLS |
| | | | EESVIAVGRLGWQKGFDFLIDSWVLVDDKHPDWHLD |
| | | | IFGEGPDRLELQHQIDRKGLHDKVRLCGVTKQIEEEY |
| | | | GKHSIYVMSSRAEGFPLALLEASSCGLPMISFNCHQGP |
| | | | NEIIQEGENGFLVDKVGDIYTLSDRICKLIEDNNLRNM |
| | | | MGKKALDSSFRFEGEVIKKDWISLLKQLI |
| 13 | Cluster: Protein | A0A096B759 | MKRLFFMFLFLGTITMNSLAQEEKPIKYETKNFSLPDK |
| | TonB | | MPLYPGGDGALRAFLSLNLHYPEKAQAFGVEGRSLM |
| | | | KFCVSSDGSIKDISAVDCKITNYNRTEFNKLPLSKQESL |
| | | | KKECAKAFAKEAARVIRLMPKWEPAELNGKKMNVY |
| | | | YSLPFTFKLR |
| 14 | Cluster: | G6AEN6 | MNYPLFIARKIYNGGDRTRKVSKPAIRIATIGVAIGLA |
| | Uncharacterized | | VMIISVGVVLGFKHTIRNKVVGFGSDITVANFLTLQSS |
| | protein | | EQYPIQITDSLVKSLQITPGIKHVQRYDYTQGILKTDN |
| | | | DFLGVLLKGVGPDFDSTFIHENMVEGSLPHFHDNESQ |
| | | | QKIVISKTIADKLNLKVGQRIFAYFINKQGVRTRKFTIT |
| | | | GIYATNMKQFDSQICFTDIYTTNKLNGWEPDQYSGAE |
| | | | LQVDNFSQLTPISMRVLNKVKNTVDHYGGTYSSENIIE |
| | | | QNPQIFSWLDLMDMNVWIILALMISVAGVTMISGLLIII |
| | | | LERTQMIGILKALGSRNRQIRHIFLWFATFIIGKGLLWG |
| | | | NIIGLGCILFQSWTGLVKLDPQTYYVNTVPVEINIPLIIA |
| | | | LNMVTMLVCLVILIAPSYLISHIHPAKSMHYE |
| 15 | Bifunctional_(p)pp | P9WHG9 | MEDKFIYTDKERKLSYQILDELKDTLDKSFLENDLPM |
| | Gpp_synthase/ | | LQVQLKDSVAKNTIHRNVFGLNPILCSLQTAAIAVKDI |
| | hydrolase_RelA | | GLKRDSVIAILLHQSVQDGYITLEDIDNRFGKSVAKIIH |
| | | | GLIRIQTLYQKNPIIESENFRNLLLSFAEDMRVILIMIAD |
| | | | RVNLMRQIRDAEDKEAQHKVAEEASYLYAPLAHKLG |
| | | | LYQLKRELEDLSLKYLEHDAYYLIKDKLNATKASRDA |
| | | | YINQFIAPVRERLTAGGLRFHIKGRTKSIHSIWQKMKK |
| | | | QKCGFEGIYDLFAIRIILDAPLEKEKIQCWQAYSIVTD |
| | | | MYQPNPKRLRDWLSVPKSNGYECLHITVLGPEKKWV |
| | | | EVQIRTERMDEIAEHGLAAHWRYKGIKEEGGLDDWL |
| | | | ASIRAALEAGDNLEVMDQFKSDLYEKEIYVFTPKGDL |
| | | | LKFPKGATILDFAYHIHSKVGNQCVGGKINAKNVSLR |
| | | | TELHSGDTVEILTSATQKPKAEWLKIVKSSRAKAKIRL |
| | | | ALKETQIKDGLYAKELLERRFKNKKIEIEESTMGHLLR |
| | | | KLGFKEVSEFYKQVADEKLDPNYIIEEYQKVYNHDHN |
| | | | LNQPKETESAENFEFENPTNEFLKKNDDVLVIDKNLK |
| | | | GLDFSLAKCCHPIYGDPVFGFVTVNGGIKIHRTDCPNA |
| | | | PEMRKRFGYRIVKARWSGKGSSQYAITLRVIGNDDIGI |
| | | | VSNITNVISKDEKIVMRSINIDSHDGLFSGNLVVLLDD |
| | | | NSKLNMLIKKLRTVKGVKQVTRI |
| 16 | Vitamin_B12_import_ | P06609 | MKRRIFLFVALSVSIVILFGLNLIIGSVHIPLSDILTILSG |
| | system_permease_ | | SFTGKESWRFIIWDSRLPQALTAMLCGSSLAVCGLML |
| | protein_BtuC | | QTAFRNPLAGPDVFGISSGASLGVALVMLLLGGTVET |
| | | | SMFTASGFLAILIVAFAGAILVTAFILFLSSVVRNSVLL |
| | | | LIVGIMVGYVASSAVTLLNFFSSEDGVKGYIVWGMGN |
| | | | FGGVSMSHIPLFAFLCLAGIIASFLLVKPLNILLLGPQY |
| | | | AESLGISIRRIRNILLVVVGILTAVTTAFCGPISFIGLAAP |
| | | | HVARLLFRTENHQKLLPGTLLVGTVVALLCNLICFLPR |
| | | | ESGMIPLNAVTPLIGAPIIIYVIMKRH |
| 17 | NADH- | P33599 | MKLENKEFGFDSFATEMARLKNEKHFDYLVTVVGED |
| | quinone_oxidoreductase_ | | FGTEEGLGCIYILENTSTHERCSVKQLAKKVGEEFVIPS |
| | subunit_C/D | | VIKLWADADLLEREVYDFYGIKFLGHPDMRRLFLRND |
| | | | FKGYPLRKDYDMDPAKNMYTTEDDVELDTTTEWNL |
| | | | DKNGELVGTQHALFTDDNFVVNIGPQHPSTHGVLRLQ |
| | | | TVLDGETVTNIYPHLGYIHRGIEKLCEQFTYPQTLALT |
| | | | DRMNYLSAMMNRHALVGVIEEGMGIELSERILYIRTI |
| | | | MDELQRIDNHLLYTACCAQDLGALTAFLYGMRDREH |
| | | | VLNVMEETTGGRLIQNYYRIGGLQADIDPNFVSNVKE |
| | | | LCKYLRPMIQEYVDVFGDNVITHQRFEGVGVMDEKD |
| | | | CISYGVTGPAGRASGWKNDVRKYHPYAMYDKVNFE |
| | | | EITLTNGDSMDRYFCHIKEIYQSLNIIEQLIDNIPEGEFY |
| | | | IKQKPIIKVPEGQWYFSVEGASGEFGAYLDSRGDKTA |
| | | | YRLKFRPMGLTLVGAMDKMLRGQKIADLVTTGAAL |
| | | | DFVIPDIDR |
| 18 | FKBP- | P45523 | MRTSTQSKDMGKKQEYKLRNEEFLHNISKKDSIKTLP |
| | type_peptidyl- | | HGIFYEIIKEGSGEGTVQPRSIVICNYRGSLISGQVFDDS |
| | prolyl_cis- | | WQKPTPEAFRLNELITGLQIALCAMHKGDSWRIYIPY |
| | trans_isomerase | | QEGYGSKRNADIPAFSTLIFDIELINIA |
| 19 | Putative acetolactate_ | P9WKJ3 | MADNKIAKESVKREVIAGERLYTLLVYSENVAGVLN |
| | synthase_small_ | | QIAAVFTRRQVNIESLNVSASSIEGIHKYTITAWSDAAT |
| | subunit | | IEKITKQVEKKIDVIKADYYEDSDLFIHEVGLYKIATPI |
| | | | LLENAEVSRAIRKRNARMMEVNPTYSTVLLAGMTDE |
| | | | VTALYHDLKNFDCLLQYSRSGRVAVTRGFSEPVSDFL |
| | | | KSEEESSVL |
| 20 | Serine/threonine_ | P0AGE4 | MKKKVKIGLLPRVIIAILLGIFFGYFMPTPLARVFLTFN |
| | transporter_SstT | | GIFSQFLGFMIPLIIIGLVTPAIADIGKGAGKLLLVTVIIA |
| | | | YVDTVVAGGLAYGTGLCLFPSMIASTGGAMPHIDKA |
| | | | TELAPYFSINIPAMADVMSGLVFSFMLGLGIAYGGLTA |
| | | | TKNIFNEFKYVIEKVIAKAIIPLLPLYIFGVFLNMAHNG |
| | | | QAQQILLVFSQIIIVILVLHVFILVYQFCIAGAIIRRNPFR |
| | | | LLWNMMPAYLTALGTSSSAATIPVTLEQTMKNGVGK |
| | | | EIAGFVVPLCATIHLSGSAMKITACALTICLLVGLPHDP |
| | | | ALFIYFILMLSIIMVAAPGVPGGAIMAALAPLASILGEN |
| | | | SEAQALMIALYIAMDSFGTACNVTGDGAIALVVNKM |
| | | | FGKKER |
| 21 | Cluster: | G6AJ07 | MKKLLLLVCAAVMSLSASAQAGDKALGAQLVFGSET |
| | Uncharacterized | | NSLGFGVKGQYYFTDHIRGEGSFDYFLKNKGISMWDI |
| | protein | | NANVHYLFDVADKFKVYPLAGLGYTNWSYKYEYAG |
| | | | APVVEGSDGRLAVNLGGGVEYELTKNLNVNAEAKY |
| | | | QIISNYNQLVLGVGVAYKF |
| 22 | Heterocyst_different | P22638 | MHFYCTKSSLDTMSERYVKRMIAKLASQGKTVISIAH |
| | iation ATP- | | RFSTIMDAKHIILLAKGKVVAEGTHQELLKTSEDYRK |
| | binding protein | | LWSDQNDEID |
| 23 | UDP-2,3- | Q912V0 | MKNVYFLSDAHLGSLAIAHRRTQERRLVRFLDSIKHK |
| | diacylglucosamine | | ASAVYLLGDMFDFWDEYKYVVPKGFTRFLGKVSELT |
| | hydrolase | | DMGVEVHFFTGNHDLWTYGYLEEECGVILHRKPVTM |
| | | | EIYGKVFYLAHGDGLGDPDPMFQFLRKVFHNRVCQR |
| | | | LLNFFHPWWGMQLGLNWAKKSRLKRADGKEMPYLG |
| | | | EDKEYLVRYTKDYMRSHKDIDYYIYGHRHIELDLTLS |
| | | | GKVRMLILGDWIWQFTYAVFDGEHMFLEEYIEGESKP |
| 24 | Anaerobic_glycerol- | P0A9C0 | MNSKQNDNYDVIIIGGGITGAGTARDCALRGLKVLLV |
| | 3- | | EKFDFTNGATGRNHGLLHSGARYAVTDPESATECIKE |
| | phosphate_dehydrogenase | | NMVLRRIAKHCIEETDGLFITLPEDDINYQKTFVEACA |
| | | | RAGISANIISPEEALRLDPSVNPDLLGAVRVPDASVDPF |
| | | | HLTTANVLDARQHGADVLTYHEVVAILTSNGRVEGV |
| | | | RLRNNHTGEEIEKHAVLVINAAGIWGHDIAKMADIKI |
| | | | NMFPAKGTLLVFGHRVNKMVINRCRKPANADILVPD |
| | | | DAVCVIGTTSDRVPYDTVDNLKITSEEVDTLIREGEKL |
| | | | APSLATTRILRAYAGVRPLVAADNDPTGRSISRGIVCL |
| | | | DHEKRDGLTGMITITGGKMMTYRLMAEQATDLACK |
| | | | KLGINKTCETATTPLPGTAGKDSDNPHHTYSTAHKAA |
| | | | KGRQGNRVKEIDERTEDDRALICECEEVSVGEAKYAI |
| | | | EELHVHDLLNLRRRTRVGMGTCQGELCACRAAGVM |
| | | | CENGVKVDKAMTDLTKFINERWKGMRPVAWGSTLD |
| | | | EAQLTTIIYQGLCGLGI |
| 25 | Anaerobic_glycerol- | P13033 | MRYDTIIIGGGLSGLTAGITLAKAGQKVCIVSAGQSSL |
| | 3- | | HFHSGSFDLLGYDADGEVVTHPLQAIADLKAEHPYSK |
| | phosphate_dehydrog | | IGISNIEHLASQAKTLLCEAGISVMGNYEQNHYRVTPL |
| | enase | | GTLKPAWLTTEGYAMIDDPEILPWKKVELLNIQGFMD |
| | | | FPTQFIAENLRMMGVECQIKTFTTDELSTARQSPTEMR |
| | | | ATNIAKVLANKDALSKVSERINAISGDPDALLLPAVLG |
| | | | FSNAESLDEMKQWIKKPVQYIATLPPSVSGVRTTILLK |
| | | | RLFAQAGGTLLIGDSATTGQFSGNHLVSITTDHLPDEK |
| | | | LYADHFILASGSFMSHGIRSNYAGVYEPVFKLDVDAA |
| | | | EKRDDWSVTNAFEAQPYMEFGVHTDKDFHATKDGK |
| | | | NIENLYAIGSVLSGHNSIKHADGTGVSLLTALYVAKKI |
| | | | TGKG |
| 26 | Anaerobic_glycerol- | POA996 | MAEGIQLKNISGNNLEQCLKCSICTAYCPVSAVEPKYP |
| | 3- | | GPKQSGPDQERYRLKDSKFFDEALKMCLNCKRCEVA |
| | phosphate_dehydrogenase | | CPSGVRIADIIQASRITYSTHRPIPRDIMLANTDFVGTM |
| | | | ANMVAPIVNATLGLKPVKAVLHGVMGIDKHRTFPAY |
| | | | SSQKFETWYKRMAAKKQDSYSKHVSYFHGCYVNYN |
| | | | FPQLGKDLVKIMNAVGYGVHLLEKEKCCGVALIANG |
| | | | LSGQARRQGKVNIRSIRKAAEQNRIVLTTSSTCTFTMR |
| | | | DEYEHLLDIKTDDVRENITLATRFLYRLIEKGDIKLAF |
| | | | RKDFKMRTAYHSACHMEKMGWIIYSTELLKMIPGLE |
| | | | LIMLDSQCCGIAGTYGFKKENYQRSQEIGEGLFKQIKE |
| | | | LNPDCVSTDCETCKWQIEMSTGYEVKNPISILADALD |
| | | | VEETIKLNQ |
| 27 | Glycerol_uptake_ | P18156 | MMIKNIVLSIPISLIIYLNHLIMEYSMTTQFLMELIGTLI |
| | facilitator_protein | | LVLFGDGVCACVTLNKSKGQKAGWVVITIAWGLAVC |
| | | | MGVLVAGPYTGAHLNPAVSIGLAVAGMFPWSSVPYY |
| | | | IVAQMIGGFLGGLLVWFFYKDHYDATDDEAAKLGTF |
| | | | CTSPAIRNYKMNFLSEVIATLVLVFIIISFSVDGNTGDA |
| | | | EHFKFGLAALGPIPVTLLIIALGMSLGGTTGYAMNPAR |
| | | | DLSPRLAHAVCMKGDNDWSYSWIPVLGPIIGAIIAGFC |
| | | | GAALLLV |
| 28 | Serine/threonine- | Q97PA9 | MSEKIIPSNEPAQAASEPIKASYTEYTVIPSQGYCQFVK |
| | protein_kinase_StkP | | CKKGDQPVVLKGLKEAYRERVLLRNALKREFKQCQR |
| | | | LNHPGIVRYQGLVDVEGYGLCIEEEYVDGRTLQAYLK |
| | | | ESHTDDEKITIVNQIADALRYAHQQGVAHRNLKPSNIL |
| | | | ITKQGDHVKLIDFNVLSLDDVKPTADTTRFMAPELKD |
| | | | ETMTADGTADIYSLGTIMKVMGLTLAYSEVIKRCCAF |
| | | | KRSDRYSDIDEFLADFNHDGSSFSMPKIGKGTVVIGFI |
| | | | AVVVIALAALAYNYGGALVDQVGKIDVTSIFKSDAET |
| | | | APEDSAMVKSVEQNNNDSVADEAPATGKLAFMNTM |
| | | | KPALYKDLDRLFAKHSDDRAKLNRAIKVYYRGLIQA |
| | | | NDTLDNEQRAELDRVFGNYVKQKKAALK |
| 29 | Cluster: D-alanyl-D- | G6AHI1 | MLVAQLFVGVLQAQKPVQNRRQAVGQSMERQGLVN |
| | alanine dipeptidase | | VKAVVPSIKVALMYARTDNFCHRMALS |
| 30 | Anaerobic C4- | POABN5 | MITGLVIIQLLIVLALIFIGARVGGIGLGIYGMIGVFILV |
| | dicarboxylate_ | | YGFGLAPGSAPIDVMMIIVAVITAASALQASGGLEYLV |
| | transporter_DcuA | | GVAAKFLQKHPDHITYFGPITCWLFCVVAGTAHTSYS |
| | | | LMPIIAEIAQTNKIRPERPLSLSVIAASLGITCSPVSAAT |
| | | | AALISQDLLGAKGIELGTVLMICIPTAFISILVAAFVEN |
| | | | HIGKELEDDPEYKRRVAAGLINPEAACEEVQKAENEH |
| | | | DPSAKHAVWAFLFGVALVILFGFLPQLRPEGVSMSQTI |
| | | | EMIMMSDAALILLVGKGKVGDAVNGNIFKAGMNAV |
| | | | VAIFGIAWMGNTFYVGNEKILDAALSSMISSTPILFAV |
| | | | ALFLLSIMLFSQAATVTTLYPVGIALGINPLLLIAMFPA |
| | | | CNGYFFLPNYPTEVAAIDFDRTGTTRVGKYVINHSFQI |
| | | | PGFITTIVSILLGVLMVQFFR |
| 31 | L-asparaginase_2 | P00805 | MRILKITFVTVLALVMSTVVFAQKPKIRIIATGGTIAGV |
| | | | SASATSSAYGAGQVGVQTLIDAVPQIKDIADVSGEQL |
| | | | VNIGSQDMNDEVWLKLAKRINDLLNKEGYDGVLITH |
| | | | GTDTMEETAYFLSLTVHTDKPVVMVGSMRPSTAISAD |
| | | | GPANLYNGICTLVDPSSKGHGVMVCMNNELFEAKSVI |
| | | | KTHTTDVSTFKGGLYGEMGYVYNGKPYFLHKPVAKQ |
| | | | GLTSEFNVDNLTSLPKVGIVYGYANCSPLPIQAFVNAK |
| | | | FDGIVLAGVGDGNFYKDVFDVALKAQNSGIQIVRSSR |
| | | | VPFGPTNLNGEVDDAKYHFVASLNLNPQKARVLLML |
| | | | ALTKTKDWQKIQQYFNEY |
| 32 | Trehalose_synthase/ | P9WQ19 | MALACAMTMSASAQMGTNPKWLGDAIFYQIYPSSY |
| | amylase_TreS | | MDTDGNGIGDLPGITQKLDYIKSLGVNAIWLNPVFES |
| | | | GWFDGGYDVIDFYKIDPRFGTNTDMVNLVKEAHKRG |
| | | | IKVCLDLVAGHTSTKCPWFKESANGDRNSRYSDYFIW |
| | | | TDSISEADKKEIAERHKEANPASSTHGRYVEMNAKRG |
| | | | KYYEKNFFECQPALNYGFAKPDPNQPWEQPVTAPGP |
| | | | QAVRREMRNIMAFWFDKGVDGFRVDMASSLVKNDW |
| | | | GKKEVSKLWNEMREWKDKNYPECVLISEWSDPAVAI |
| | | | PAGFNIDFMIHFGIKGYPSLFFDRNTPWGKPWPGQDIS |
| | | | KDYKFCYFDKAGKGEVKEFVDNFSEAYNATKNLGYI |
| | | | AIPSANHDYQRPNIGTRNTPEQLKVAMTFFLTMPGVP |
| | | | FIYYGDEIGMKYQMDLPSKEGSNERAGTRTPMQWTS |
| | | | GPTAGFSTCNPSQLYFPVDTEKGKLTVEAQQNDPRSL |
| | | | LNYTRELTRLRHSQPALRGNGEWILVSKESQPYPMVY |
| | | | KRTSGGETVVVAINPSDKKVSANIAHLGKAKSLIMTG |
| | | | KASYKTGKTEDAVELNGVSAAVFKIAE |
| 33 | Ribitol-5- | Q720Y7 | MNIAVIFAGGSGLRMHTKSRPKQFLDLNGKPIIIYTLEL |
| | phosphate_cytidylyl | | FDNHPGIDAIVVACIESWIPFLEKQLRKFEINKVVKIVP |
| | transferase | | GGESGQASIYNGLCAAEAYIKSKNVASEDTTVLIHDG |
| | | | VRPLITEETITDNINKVAEVGSCITCIPATETLVVKQHD |
| | | | GSLEIPSRADSLIARAPQSFLLSDILTAHRRAIDEKKND |
| | | | FIDSCTMMSHYGYRLGTIIGPMENIKITTPTDFFVLRA |
| | | | MVKVHEDQQIFGL |
| 34 | UDP-Glc:alpha-D- | B5L3F2 | MTEKKSVSIVLCTYNGTKYLQEQLDSILAQTYPLHEIII |
| | GlcNAc- | | QDDGSTDNTWQILEKYEEKYPLIHIYHNEGTHGVNAN |
| | diphosphoundecaprenol | | FLSAMHRTTGDFIAIADQDDIWETDKIANQMTTIGNK |
| | | | LLCSGLTRPFSSDGSFAYFDNRPRNVSIFRMMFLGLPG |
| | | | HTMLFRRELLRMMPPVTHSFFNVSLYDAALSILAASH |
| | | | DSIAFCNKVLVNFRRHADATTYNDYSRSLPSWQNGL |
| | | | YELLWGLRHYHQARSIALPIYRGKLALMEGITTNYHD |
| | | | FIEAKAIMRLETQKGLWAFLRLQYLLTKNHQRLFQTS |
| | | | GGSFIKMIRAWLYPVMQLYMYHHALRRCK |
| 35 | UDP-N- | P33038 | MESFIIEGGHRLSGTIAPQGAKNEALEVICATLLTTEEV |
| | acetylglucosamine | | IIRNIPNILDVNNLIKLLQDIGVKVKKLGANDFSFQADE |
| | | | VKLDYLESIDFVKKCSSLRGSVLMIGPLLGRFGKATIA |
| | | | KPGGDKIGRRRLDTHFLGFKNLGARFVRIEDRDVYEI |
| | | | QADKLVGDYMLLDEASVTGTANIIMSAVMAEGTTTI |
| | | | YNAACEPYIQQLCHLLNAMGAKITGIASNLITIEGVTS |
| | | | LHGAEHRILPDMIEVGSFIGMAAMVGDGVRIKDVSIP |
| | | | NLGLILDTFRRLGVQIIEDEDDLIIPRQDHYVIDSFIDGT |
| | | | IMTISDAPWPGLTPDLISVLLVVATQAQGSVLFHQKM |
| | | | FESRLFFVDKLIDMGAQIILCDPHRAVVVGHDHAKKL |
| | | | RAGRMSSPDIRAGIALLIAALTAEGTSRIDNIAQIDRGY |
| | | | ENIEGRLNALGAKVQRVEIC |
| 36 | Sensor_protein_EvgS | P30855 | MERSGNFYKAIRLGYILISILIGCMAYNSLYEWQEIEAL |
| | | | ELGNKKIDELRKEINNINIQMIKFSLLGETILEWNDKDI |
| | | | EHYHARRMAMDSMLCRFKATYPAERIDSVRHLLEDK |
| | | | ERQMCQIVQILEQQQAINDKITSQVPVIVQKSVQEQPK |
| | | | KSKRKGFLGIFGKKEEAKPTVTTTMHRSFNRNMRTEQ |
| | | | QAQSRRLSVHADSLAARNAELNRQLQGLVVQIDGKV |
| | | | QTDLQKREAEITAMRERSFIQIGGLTGFVILLLVISYIII |
| | | | HRNANRIKRYKQETADLIERLQQMAKRNEALITSRKK |
| | | | AVHTITHELRTPLTAITGYAGLIQKNFNADKTGMYIRN |
| | | | IQQSSDRMREMLNTLLSFFRLDDGKEQPNFSTCRISSIA |
| | | | HTLESEFMPIAINKGLALTVTNHTDAVVLTDKERILQI |
| | | | GNNLLSNAIKFTENGAVSLTMGYDNGMLKLIVKDTG |
| | | | SGMTEEEQQRVFGAFERLSNAAAKDGFGLGLSIVQRI |
| | | | VTMLGGTIQLKSEKGKGSRFTVEIPMQSAEELPERINK |
| | | | TQIHHNRTLHDIVAIDNDKVLLLMLKEMYAQEGIHCD |
| | | | TCTNAAELMEMIRRKEYSLLLTDLNMPDINGFELLEL |
| | | | LRTSNVGNSRIIPIIVTTASGSCNREELLERGFSDCLLKP |
| | | | FSISELMEVSDKCAMKGKQNEKPDFSSLLSYGNESVM |
| | | | LDKLIAETEKEMQSVRDGEQRKDFQELDALTHHLRSS |
| | | | WEILRADQPLRELYKQLHGSAVPDYEALNNAVTAVL |
| | | | DKGSEIIRLAKEERRKYENG |
| 37 | Phosphate- | Q7A5Q2 | MKRSRFYITVGLILSLTLLMSACGQKKAKDGRTDTPT |
| | binding protein_PstS | | SGTIKFASDESFSPIVEELLQNYQFRYPQAHLLPIYTDD |
| | | | NTGMKLLLDQKVNLFITSHAMTKGEDAILRGKGPIPE |
| | | | VFPIGYDGIAFIVNRSNPDSCITVDDVKKILQGKIAKW |
| | | | NQLNPKNNRGSIEVVFDNKASATLHYVVDSILGGKNI |
| | | | KSENIVAAKNSKSVIDYVNKTPNAIGVIGSNWLNDHR |
| | | | DTTNTTFKKDVTVASISKATVASPSNSWQPYQAYLLD |
| | | | GRYPFVRTIYALLADPHKALPYAFANYIANPIGQMIIF |
| | | | KAGLLPYRGNINIREVEVKNQ |
| 38 | Bifunctional_purine | P9WHM7 | MAGTKRIKTALISVFHKDGLDDLLKKLDEEGVQFLST |
| | biosynthesis_protein_ | | GGTQQFIESLGYECQKVEDVTSYPSILGGRVKTLHPKI |
| | PurH | | FGGILARRDNEEDQKQMVEYTIPAIDLVIVDLYPFEQT |
| | | | VASGASAQDIIEKIDIGGISLIRAGAKNFKDVVIVPSKA |
| | | | EYPVLLQLLNTKGAETEIEDRKMFAERAFGVSSHYDT |
| | | | AIHSWFAAE |
| 39 | Multidrug_efflux_ | POAE06 | MEEEKGGRIGQRPYILKIITERNYIIIIDMKKAKILLFVT |
| | pump_subunit_AcrA | | ALVAVLTSCGGGQKGLPTSDEYPVITIGASNAQLKTT |
| | | | YPATIKGVQDVEVRPKVSGFITKLNIHEGEYVHAGQV |
| | | | LFVIDNSTYQAAVRQAQAQVNSAQSAVAQAKANVV |
| | | | QANASLNSANAQAATSRLTYNNSQNLYNNKVIGDYE |
| | | | LQSAKNTYETAQASVRQAQSGIASAQAAVKQAEAGV |
| | | | RQAQAMLSTAKDNLGFCYVKSPASGYVGSLPFKEDA |
| | | | LVSASSAQPVTTISNTSTIEVYFSMTEADVLKLSRTDD |
| | | | GLSNAIKKFPAVSLLLADGSTYNHEGAIVKTSGMIDAT |
| | | | TGTINVIARFPNPEHLLKSGGSGKIVIAKNNNRALLIPQ |
| | | | EAVTQVQNKMFVYKVDAKDKVHYSEITVDPQNDGIN |
| | | | YIVTSGLKMGERIVSKGVSSLEDGAKIKALTPAEYEEA |
| | | | IKKAEKLGENQSSASGFLKTMKGDSK |
| 40 | Cell_division_protein_ | Q81X30 | MAKRRNKARSHHSLQVVTLCISTAMVLILIGMVVLTV |
| | FtsX | | FTSRNLSSYVKENLTVTMILQPDMSTEESAALCQRIRS |
| | | | LHYINSLNFISKEQALKEGTRELGANPAEFAGQNPFTG |
| | | | EIELQLKANYANNDSIKNIERELRTYRGVSDITYPQNL |
| | | | VESVNHTLGKISLVLLVIAILLTIVSFSLMNNTIRLSIYA |
| | | | RRFSIHTMKLVGASWGFIRAPFLRRAVMEGLVSALLA |
| | | | IAVLGVGLCLLYDYEPDITKVLSWDVLVITAGVMLAF |
| | | | GVLIATFCSWLSVNKFLRMKAGDLYKI |
| 41 | Fe(2+)_transporter_ | Q9PMQ9 | MKLSDLKTGETGVIVKVLGHGGFRKRIIEMGFIQGKQ |
| | FeoB | | VEVLLNAPLRDPVKYKIMGYEVSLRHSEADQIEVISAE |
| | | | EARQLEQAKADNEPQQGALSNNIPDESDHALTPFELT |
| | | | DAANRKSKVINVALVGNPNCGKTSLFNFASGAHERV |
| | | | GNYSGVTVDAKVGRANYEGYEFHLVDLPGTYSLSAY |
| | | | SPEELYVRKQLVEKTPDVVINVIDASNLERNLYLTTQL |
| | | | IDMHVRMVCALNMFDETEQRGDNIDYQKISELFGIPM |
| | | | VPTVFTNGRGVKELFHQVIAVYEGKEDETSQFRHIHIN |
| | | | HGHELEGGIKNIQEHLRAYPDICQRYSTRYLAIKLLEH |
| | | | DKDVEELIKPLKDSDEIFKHRDIAAQRVKEETGNESET |
| | | | AIMDAKYGFIHGALEEADYSTGQKKDTYQTTHFIDQI |
| | | | LTNKYFGFPIFFLILFIMFTATFVIGQYPMDWIDGGVS |
| | | | WLGDFISSNMPDGPVKDMLVDGIIGGVGAVIVFLPQIL |
| | | | ILYFFISYMEDSGYMARAAFIMDKLMHKMGLHGKSFI |
| | | | PLIMGFGCNVPAVMATRTIESRRSRLVTMLILPLMSCS |
| | | | ARLPIYVMITGSFFALKYRSLAMLSLYVIGILMSVIMS |
| | | | RVFSRFLVKGEDTPFVMELPPYRFPTWKAIGRHTWEK |
| | | | GKQYLKKMGGIILVASIIVWALGYFPLPDKPDMGQQE |
| | | | RQEHSFIGQIGHAVEPVFRPQGFNWKLDVGLLAGVGA |
| | | | KEIVASTMGVLYSNDDSFKDDNSFSSEGGKYVKLHK |
| | | | QITQDVANLHGVSYNEAEPIATLTAFCFLLFVLLYFPCI |
| | | | ATIAAIKGETGSWGWALFAAGYTTLLAWVVSAIVFQ |
| | | | VGMLFIG |
| 42 | Pneumolysin | Q04IN8 | MKKNLLKAVLPASLALFAVTFGSCSQDGQLTGTKED |
| | | | TGERVLDNTREIQNYLRTLPLAPMMSRASDPVPSDDG |
| | | | TTVPVDEGTSKTEEKGVLNGIPGSWVKTTRRYKMTQ |
| | | | AFDESFLFDPTSDIVYPGCVLKGGTIANGTYAIITSHET |
| | | | GDVTFSINLSPANPQEARETSATVHNIRKSEYQEVWN |
| | | | KWANMQWKESPITTIESVEKINSQEELATKLGVAVNS |
| | | | PVANGSLNFGFNFNKKKNHILARLIQKYFSVSTDAPK |
| | | | KGNIFESIDKEALDGYQPVYISNINYGRIIYLSVESDED |
| | | | EKVVDEAINFAMNQIKGVDVSVSADQSLHYRKVLAN |
| | | | CDIRITVLGGGQTIQKEVLKGDIDSFQRFLNADIPMEQ |
| | | | MSPISFSLRYAVDNSQARVVTSNEFTVTQRDFVPEFKK |
| | | | VRMQLQVLGFSGTNTGPFPNLDREAGLWGSISLSLNG |
| | | | QDNELVKISQSNPFFFNYREKKETMHPIGFGGIVTVEF |
| | | | DKDPNESLEDFVDHQKMTFVSDLHSTRSIYNYNFGRT |
| | | | TFTHTLGTLYTKYKGDDPIFVLESNNKNVKIHTYVKV |
| | | | LDMKFFN |
| 43 | Cluster: | G6AG77 | MTKFIYAMSLFLLAAISIKAQPIQKTSGCLLHGSVVSST |
| | Uncharacterized | | DATAIAGATVRLYQLKKLVGGTVSDASGNFDVKCPSS |
| | protein | | GSLQLRITAVGFKEVDTTLNVPTVTPLSIYMRAGKHA |
| | | | MDEVTVTASEKRGMTSTTVIGQTAMEHLQPSSFADLL |
| | | | ALLPGGMTKIPALGSANVITLREAGPPSSQYATSSLGT |
| | | | KFVIDGQAIGTDANMQYIAGSFQGDADNSRNHVSYG |
| | | | VDMREIPTDNIEKVEVVRGIPSVKYGELTSGLINITRKR |
| | | | SQSPLLLRLKADEYGKLVSVGKGFLLSGKWNLNVDG |
| | | | GLLDARKEPRNRFETYRRLTFSARLRRKWNLGERYVL |
| | | | EWSGATDYSLNIDNVKTDPEIQIHREDSYRSSYLKMG |
| | | | MNHRLLLRRKALVGLQSVSLAYSASLASDRIHQTEAV |
| | | | ALQRDYVVPLAYEGGEYDGLFLPMQYLCDYRVEGKP |
| | | | FYSTLRGETEWLARTSFISHHITAGGEFLLNKNYGRGQ |
| | | | IFDITKPLHASTARRPRSYKDIPATDILSFYAEDKATMP |
| | | | IGKHQLTVMAGLRTTQMLNIPASYAVHGKLFTDTRV |
| | | | NVQWDFPSFLGFKSFVSGGLGMMTKMPTVLDLYPDY |
| | | | VYKDITEMNYWDIRPAYKRIHIRTYKLNQVNPDLRPA |
| | | | RNKKWEIRLGMDKGAHHFSVTYFHEDMKDGFRSTTT |
| | | | MRPFIYKRYDTSVINPSALTGPPSLASLPVVTDTLLDG |
| | | | YGRTENGSRITKQGIEFQYSSPRIPVIQTRITVNGAWFR |
| | | | TLYENSIPLFRSAPNVVVGTVAIADRYAGYYMSTDKY |
| | | | DKQIFTSNFIFDSYVDKLGLILSATAECFWMSNTKRPA |
| | | | TSSTPMGYMDITGTVHPYVEADQSDPYLRWLVLTGT |
| | | | AGQDMDYRERSYMLVNFKATKRFGRHLSLSFFADRV |
| | | | FYVAPDYEVNGFIVRRTFSPYFGMEIGLKI |
| 44 | Cell_division ATP- | P0A9R7 | MLIDFKKVNIYQDERLILKDIDFQATEGEFIYLIGRVGS |
| | binding protein_FtsE | | GKSSLLKTFYGELDIDQEDAEKAEVLGESVLDIKQKRI |
| | | | PALRRQMGIIFQDFQLLHDRSVAKNLKFVLQATGWK |
| | | | DKEKIKQRIKEVLEQVGMIDKAAKMPSELSGGEQQRI |
| | | | AIARAFLNNPKIILADEPTGNLDPETASNIVSILKDTCK |
| | | | NGTTVIMSTHNINLLSQFPGKVYRCMEQALVPVTNEA |
| | | | QTKDLEEDSTSVEPLIEPVLEEEAQAEDSKE |
| 45 | Di- | P0C2U3 | MFENQPKALYALALANTGERFGYYTMIAVFALFLRA |
| | /tripeptide_transporter | | NFGLEPGTAGLIYSIFLGLVYFLPLIGGIMADKFGYGK |
| | | | MVTIGIIVMFAGYLFLSVPLGGGTVAFGAMLAALLLIS |
| | | | FGTGLFKGNLQVMVGNLYDTPELASKRDSAFSIFYMA |
| | | | INIGALFAPTAAVKIKEWAETSLGYAGNDAYHFSFAV |
| | | | ACVSLIVSMGIYYAFRSTFKHVEGGTKKTEKAAAAAV |
| | | | EELTPQQTKERIVALCLVFAVVIFFWMAFHQNGLTLT |
| | | | YFADEFVSPTSTGVQSMAFDVVNLVMIVFIVYSIMALF |
| | | | QSKTTKAKGIACAVILAAIAVLAYKYMNVNGQVEVS |
| | | | APIFQQFNPFYVVALTPISMAIFGSLAAKGKEPSAPRKI |
| | | | AYGMIVAGCAYLLMVLASQGLLTPHEQKLAKAAGET |
| | | | VPFASANWLIGTYLVLTFGELLLSPMGISFVSKVAPPK |
| | | | YKGAMMGGWFVATAIGNILVSVGGYLWGDLSLTVV |
| | | | WTVFIVLCLVSASFMFLMMKRLEKVA |
| 46 | Calcium- | Q47910 | MKKILIFVAGLCMSLAASAQIQRPKLVVGLVVDQMR |
| | transporting_ATPase | | WDYLYYYYNEYGTDGLRRLVDNGFSFENTHINYAPT |
| | | | VTAIGHSSVYTGSVPAITGIAGNYFFQDDKNVYCCEDP |
| | | | NVKSVGSDSKEGQMSPHRLLASTIGDELQISNDFRSKV |
| | | | IGVALKDRASILPAGHAADAAYWWDTSAGHFVTSTF |
| | | | YTDHLPQWVIDFNEKNHTAPNFNIKTSTQGVTMTFK |
| | | | MAEAALKNENLGKGKETDMLAVSISSTDAIGHVYSTR |
| | | | GKENHDVYMQLDKDLAHFLKTLDEQVGKGNYLLFL |
| | | | TADHGAAHNYNYMKEHRIPAGGWDYRQSVKDLNGY |
| | | | LQGKFGIAPVMAEDDYQFFLNDSLIAASGLKKQQIIDE |
| | | | SVEYLKKDPRYLYVFDEERISEVTMPQWIKERMINGY |
| | | | FRGRSGEIGVVTRPQVFGAKDSPTYKGTQHGQPFPYD |
| | | | THIPFLLYGWNVKHGATTQQTYIVDIAPTVCAMLHIQ |
| | | | MPNGCIGTARNMALGN |
| 47 | Poly-beta-1,6-N- | Q5HKQ0 | MDRQVFQTDSRQRWNRFKWTLRVLITIAILLGVVFVA |
| | acetyl-D- | | MFALEGSPQMPFRHDYRSVVSASEPLLKDNKRAEVY |
| | glucosamine_synthase | | KSFRDFFKEQKMHSNYAKVAARQHRFVGHTDNVTQ |
| | | | KYIKEWTDPRMGIRSAWYVNWDKHAYISLKNNLKNL |
| | | | NMVLPEWYFINPKTDRIEARIDQRALKLMRRAHIPVLP |
| | | | MLTNNYNSAFRPEAIGRIMRDSTKRMGMINELVAAC |
| | | | KHNGFAGINLDLEELNINDNALLVTLVKDFARVFHAN |
| | | | GLYVTQAVAPFNEDYDMQELAKYDDYLFLMAYDEY |
| | | | NAGSQAGPVSSQRWVEKATDWAAKNVPNDKIVLGM |
| | | | ATYGYNWAQGQGGTTMSFDQTMATALNAGAKVNF |
| | | | NDDTYNLNFSYQDEDDGTLHQVFFPDAVTTFNIMRFG |
| | | | ATYHLAGFGLWRLGTEDSRIWKYYGKDLSWESAAR |
| | | | MPIAKIMQLSGTDDVNFVGSGEVLNVTSEPHAGRIGIV |
| | | | LDKDNQLIIEERYLSLPATYTVQRLGKCKEKQLVLTFD |
| | | | DGPDSRWTPKVLSILKHYKVPAAFFMVGLQIEKNIPIV |
| | | | KDVFNQGCTIGNHTFTHHNMIENSDRRSFAELKLTRM |
| | | | LIESITGQSTILFRAPYNADADPTDHEEIWPMIIASRRN |
| | | | YLFVGESIDPNDWQQGVTADQIYKRVLDGVHQEYGH |
| | | | IILLHDAGGDTREPTVTALPRIIETLQREGYQFISLEKYL |
| | | | GMSRQTLMPPIKKGKEYYAMQANLSLAELIYHISDFL |
| | | | TALFLVFLVLGFMRLVFMYVLMIREKRAENRRNYAPI |
| | | | DPLTAPAVSIIVPAYNEEVNIVRTISNLKEQDYPSLKIY |
| | | | LVDDGSKDNTLQRVREVFENDDKVVIISKKNGGKAS |
| | | | ALNYGIAACSTDYIVCVDADTQLYKDAVSKLMKHFIA |
| | | | DKTGKLGAVAGNVKVGNQRNMLTYWQAIEYTTSQN |
| | | | FDRMAYSNINAITVIPGAIGAFRKDVLEAVGGFTTDTL |
| | | | AEDCDLTMSINEHGYLIENENYAVAMTEAPESLRQFI |
| | | | KQRIRWCFGVMQTFWKHRASLFAPSKGGFGMWAMP |
| | | | NMLIFQYIIPTFSPIADVLMLFGLFSGNASQIFIYYLIFLL |
| | | | VDASVSIMAYIFEHESLWVLLWIIPQRFFYRWIMYYVL |
| | | | FKSYLKAIKGELQTWGVLKRTGHVKGAQTIS |
| 48 | ATP_synthase_subunit_ | P29707 | MSQINGRISQIIGPVIDVYFDTKGENPEKVLPNIYDALR |
| | beta, sodium_ion_ | | VKKADGQDLIIEVQQQIGEDTVRCVAMDNTDGLQRG |
| | specific | | LEVVPTGSPIVMPAGEQIKGRMMNVIGQPIDGMSALQ |
| | | | MEGAYPIHREAPKFEDLSTHKEMLQTGIKVIDLLEPY |
| | | | MKGGKIGLFGGAGVGKTVLIMELINNIAKGHNGYSVF |
| | | | AGVGERTREGNDLIRDMLESGVIRYGEKFRKAMDEG |
| | | | KWDLSLVDSEELQKSQATLVYGQMNEPPGARASVAL |
| | | | SGLTVAEEFRDHGGKNGEAADIMFFIDNIFRFTQAGSE |
| | | | VSALLGRMPSAVGYQPTLASEMGAMQERITSTKHGSI |
| | | | TSVQAVYVPADDLTDPAPATTFTHLDATTELSRKITEL |
| | | | GIYPAVDPLGSTSRILDPLIVGKEHYDCAQRVKQLLQK |
| | | | YNELQDIIAILGMDELSDDDKLVVNRARRVQRFLSQP |
| | | | FTVAEQFTGVKGVMVPIEETIKGFNAILNGEVDDLPEQ |
| | | | AFLNVGTIEDVKEKAKQLLEATKA |
| 49 | Cluster: | G6AGX5 | MNPIYKIITSILFCVLSINTMAQDLTGHVTSKADDKPIA |
| | Uncharacterized | | YATVILKENRLYAFTDEKGNYTIKNVPKGKYTVVFSC |
| | protein | | MGYASQTVVVMVNAGGATQNVRLAEDNLQLDEVQ |
| | | | VVAHRKKDEITTSYTIDRKTLDNQQIMTLSDIAQLLPG |
| | | | GKSVNPSLMNDSKLTLRSGTLERGNASFGTAVEVDGI |
| | | | RLSNNAAMGETAGVSTRSVSASNIESVEVVPGIASVE |
| | | | YGDLTNGVVKVKTRRGSSPFIVEGSINQHTRQIALHK |
| | | | GVDLGGNVGLLNFSIEHARSFLDAASPYTAYQRNVLS |
| | | | LRYMNVFMKKSLPLTLEVGLNGSIGGYNSKADPDRSL |
| | | | DDYNKVKDNNVGGNIHLGWLLNKRWITNVDLTAAF |
| | | | TYADRLSESYTNESSNATQPYIHTLTEGYNIAEDYDRN |
| | | | PSANIILGPTGYWYLRGFNDSKPLNYSLKMKANWSK |
| | | | AFGKFRNRLLVGGEWTSSMNRGRGTYYADMRYAPS |
| | | | WREYRYDALPSLNNIAIYAEDKLSMDVNERQNAELT |
| | | | AGIREDITSIPGSEYGSVGSFSPRMNARYVFRFGQNSW |
| | | | LNSMTLHAGWGRSVKIPSFQVLYPSPSYRDMLAFAST |
| | | | SDADNRSYYAYYTYPSMARYNANLKWQRADQWDL |
| | | | GVEWRTKIADVSLSFFRSKVSNPYMATDVYTPFTYKY |
| | | | TSPAMLQRSGIAVADRRFSIDPQTGIVTVSDASGVKSP |
| | | | VTLGYEERNTYVTNTRYVNADALQRYGLEWIVDFKQ |
| | | | IKTLRTQVRLDGKYYHYKAQDETLFADVPVGLNTRQ |
| | | | SDGRLYQYVGYYRGGAATTTNYTANASASNGSVSGQ |
| | | | VDLNATITTHIPKIRLIVALRLESSLYAFSRATSSRGYV |
| | | | VSSGNEYFGVPYDDKTENQTVIVYPEYYSTWDAPDV |
| | | | LIPFAEKLRWAETNDRGLFNDLAQLVVRTNYPYTLNP |
| | | | NRLSAYWSANLSVTKEIGRHVSVSFYANNFFNTLSQV |
| | | | HSTQTGLETSLFGSGYVPSFYYGLSLRLKI |

In some embodiments, the Prevotella bacteria is a strain of Prevotella bacteria free or substantially free of one or more (e.g., 1, 2, 3, 4, 5, 6, 7, 8, 9, 10, 11, 12, 13, 14, 15, 16, 17, 18, 19, 20, 21, 22, 23, 24, 25 or more) proteins listed in Table 2 and/or one or more (e.g., 1, 2, 3, 4, 5, 6, 7, 8, 9, 10, 11, 12, 13, 14, 15, 16, 17, 18, 19, 20, 21, 22, 23, 24, 25 or more) genes encoding proteins listed in Table 2. In some embodiments, Prevotella bacteria is free of all of the proteins listed in Table 2 and/or all of the genes encoding the proteins listed in Table 2.

TABLE 2
Other Prevotella proteins

| Seq. | | Uniprot | |
|---|---|---|---|
| ID. No. | Name | ID | Amino Acid Sequence |
| 50 | UDP-Gal:alpha-D- | Q03084 | MERIDISVLMAVYKKDNPAFLRESLESIFSQTVEAAEV |
| | GlcNAc- | | VLLEDGPLTDALYDVIKSYEAIYSTLKVVSYPENRGLG |
| | diphosphoundecaprenol | | KTLNDGLLLCKYNLVARMDADDICKPNRLEMEYNW |
| | | | LKSHEDYDVIGSWVDEFTDNKTRVKSIRKVPEAYDEI |
| | | | KNYAQYRCPINHPTAMYRKAAVLAVGGYLTEYFPED |
| | | | YFLWLRMLNNGSKFYNIQESLLWFRYSEETVAKRGG |
| | | | WAYACDEVRILVRMLKMGYIPFHVFCQSVVIRFTTRV |
| | | | MPLPIRQRLYNLIRKT |
| 51 | ATP_synthase_subunit_ | A1B8P0 | MSQINGRISQIIGPVIDVYFDTKGENPEKVLPKIHDALR |
| | beta | | VKRANGQDLIIEVQQHIGEDTVRCVAMDNTDGLQRN |
| | | | LEVVPTGSPIVMPAGDQIKGRMMNVIGQPIDGMEALS |
| | | | MEGAYPIHREAPKFEDLSTHKEMLQTGIKVIDLLEPY |
| | | | MKGGKIGLFGGAGVGKTVLIMELINNIAKGHNGYSVF |
| | | | AGVGERTREGNDLIRDMLESGVIRYGEKFRKAMDEG |
| | | | KWDLSLVDQEELQKSQATLVYGQMNEPPGARASVAL |
| | | | SGLTVAEEFRDHGGKNGEAADIMFFIDNIFRFTQAGSE |
| | | | VSALLGRMPSAVGYQPTLASEMGTMQERITSTKHGSI |
| | | | TSVQAVYVPADDLTDPAPATTFTHLDATTELSRKITEL |
| | | | GIYPAVDPLGSTSRILDPLIVGKDHYECAQRVKQLLQH |
| | | | YNELQDIIAILGMDELSDEDKLVVNRARRVQRFLSQPF |
| | | | TVAEQFTGVKGVMVPIEETIKGFNAILNGEVDDLPEQ |
| | | | AFLNVGTIEDVKEKAKRLLEATK |
| 52 | Cell_division ATP- | 005779 | MPIGNGQKYQLTIINHTEIIMLIDYKKVNIYQDERLILK |
| | binding protein_ | | DVDFQAETGEFIYLIGRVGSGKSSLLKTIYGELDIDSED |
| | FtsE | | AEKAVVLDESMPNIKRSRIPALRKQMGIIFQDFQLLHD |
| | | | RSVAKNLKFVLQATGWTSKQKIERRIEEVLAQVGMT |
| | | | DKKNKMPSELSGGEQQRIAIARALLNTPKIIIADEPTGN |
| | | | LDPETAANIVSILKDSCQAGTTVIMSTHNINLIDQFPGK |
| | | | VYRCHEGELHQLTDKKEVSELAEETAPVETIDEPEQN |
| | | | D |
| 53 | Hemin_transport_ | Q56992 | MKRNILLFICLATSILLLFGLNLTTGSVQIPFADILDILC |
| | system_permease_ | | GRFIGKESWEYIILENRLPQTLTAILCGASLSVCGLML |
| | protein_HmuU | | QTAFRNPLAGPDVFGISSGAGLGVALVMLLLGGTVST |
| | | | SIFTVSGFLAILTAAFVGAIAVTALILFLSTLVRNSVLLL |
| | | | IVGIMVGYVSSSAVSLLNFFASEEGVKSYMVWGMGN |
| | | | FGAVSMNHIPLFSILCLIGIIASFLLVKPLNILLLGPQYA |
| | | | ESLGISTRQIRNILLVVVGLLTAITTAFCGPISFIGLAIPH |
| | | | IARLLFRTENHQILLPGIVLSGAAIALLCNFICYLPGESG |
| | | | IIPLNAVTPLIGAPIIIYVIIQRR |
| 54 | Hexuronate_ | O34456 | MKKYYPWVLVALLWFVALLNYMDRQMLSTMQEAM |
| | transporter | | KVDIAELNHAEAFGALMAVFLWIYGIVSPFAGIIADRV |
| | | | NRKWLVVGSIFVWSAVTYLMGYAESFDQLYWLRAF |
| | | | MGISEALYIPAALSLIADWHEGKSRSLAIGIHMTGLYV |
| | | | GQAVGGFGATLAAMFSWHAAFHWFGIIGIVYSLVLLL |
| | | | FLKENPKHGQKSVLQGETKPSKNPFRGLSIVFSTWAF |
| | | | WVILFYFAVPSLPGWATKNWLPTLFANSLDIPMSSAG |
| | | | PMSTITIAVSSFIGVIMGGVISDRWVQRNLRGRVYTSAI |
| | | | GLGLTVPALMLLGFGHSLVSVVGAGLCFGIGYGMFD |
| | | | ANNMPILCQFISSKYRSTAYGIMNMTGVFAGAAVTQV |
| | | | LGKWTDGGNLGNGFAILGGIVVLALVLQLSCLKPTTD |
| | | | NME |
| 55 | 1,4-alpha- | P9WN45 | MVTKKTTTKKAPVKKTSAKTTKVKEPSHIGLVKNDA |
| | glucan_branching_ | | YLAPYEDAIRGRHEHALWKMNQLTQNGKLTLSDFAN |
| | enzyme_GlgB | | GHNYYGLHQTADGWVFREWAPNATEIYLVGDFNGW |
| | | | NEQEAYQCHRIEGTGNWELTLPHDAMQHGQYYKMR |
| | | | VHWEGGEGERIPAWTQRVVQDEASKIFSAQVWAPAE |
| | | | PYVWEKKTFKPQTSPLLIYECHIGMAQDEEKVGTYNE |
| | | | FREKVLPRIIKDGYNAIQIMAIQEHPYYGSFGYHVSSFF |
| | | | AASSRFGTPEELKALIDEAHKNGIAVIMDIVHSHAVKN |
| | | | EVEGLGNLAGDPNQYFYPGERHEHPAWDSLCFDYGK |
| | | | DEVLHFLLSNCKYWLEEYHFDGFRFDGVTSMLYYSH |
| | | | GLGEAFCNYADYFNGHQDDNAICYLTLANCLIHEVN |
| | | | KNAVTIAEEVSGMPGLAAKFKDGGYGFDYRMAMNIP |
| | | | DYWIKTIKELPDEAWKPSSIFWEIKNRRSDEKTISYCES |
| | | | HDQALVGDKTIIFRLVDADMYWHFRKGDETEMTHRG |
| | | | IALHKMIRLATIAAINGGYLNFMGNEFGHPEWIDFPRE |
| | | | GNGWSHKYARRQWNLVDNEELCYHLLGDFDRKMLE |
| | | | VITSEKKFNETPIQEIWHNDGDQILAFSRGELVFVFNFS |
| | | | PSHSYSDYGFLVPEGSYNVVLNTDAREFGGFGFADDT |
| | | | VEHFTNSDPLYEKDHKGWLKLYIPARSAVVLRKK |
| 56 | Cluster: YihY | D9RW24 | MKIDIERIKYFLTVGMFMKTEHSSKRRNMLIRQFQKF |
| | family protein | | YLTVKFFFVRDHAASTAQLSFSTIMAIVPIASMIFAIAN |
| | | | GFGFGQFLEKQFREMLSAQPEAATWLLKLTQSYLVH |
| | | | AKTGLFIGIGLMIMLYSVFSLIRTVETTFDNIWQVKDS |
| | | | RPISRIVIDYTALMFLVPISIIILSGLSIYFYSFVENLNGL |
| | | | RFLGTIASFSLRYLVPWAILTLMFIVLYVFMPNAKVKI |
| | | | TKTVAPAMIASIAMLCLQAVYIHGQIFLTSYNAIYGSF |
| | | | AALPLFMLWILASWYICLFCAELCYFNQNLEYYECLI |
| | | | DTEDICHNDLLILCATVLSHICQRFANDQKPQTALQIK |
| | | | TETHIPIRVMTDILYRLKEVNLISENFSPTSDEVTYTPT |
| | | | HDTNNITVGEMIARLESTPASDFALLGFSPKKAWNHDI |
| | | | YDRVGSIREIYLNELKSINIKELISYSEN |
| 57 | Capsule_biosynthesis_ | P19579 | MMKRPSIARVVKVIICLLTPILLSFSGIGDNDIDKKKST |
| | protein_CapA | | SKEVDDTLRIVITGDLLLDRGVRQKIDMAGVDALFSP |
| | | | TIDSLFHSSNYVIANLECPVTKIRERVFKRFIFRGEPEW |
| | | | LPTLRRHGITHLNLANNHSIDQGRNGLLDTQEQIKKA |
| | | | GMIPIGAGKNMEEAAEPVLISTSPRHVWVISSLRLPLE |
| | | | NFLYLPQKPCVSQESIDSLIMRVKRLRATDKNCYILLIL |
| | | | HWGWEHHFRATPQQREDAHKLIDAGADAIVGHHSHT |
| | | | LQTIETYRGKPIYYGIGNFIFDQRKPMNSRACLVELSIT |
| | | | AEKCKAKALPIEIKNCTPYLSK |
| 58 | Peptidoglycan_ | B5ZA76 | MILLSFDTEEFDVPREHGVDFSLEEGMKVSIEGTNRIL |
| | deacetylase | | DILKANNVCATFFCTGNFAELAPEVMERIKNEGHEVA |
| | | | CHGVDHWQPKPEDVFRSKEIIERVTGVKVAGYRQPR |
| | | | MFPVSDEDIEKAGYLYNSSLNPAFIPGRYMHLTTSRT |
| | | | WFMQGKVMQIPASVSPHLRIPLFWLSMHNFPEWFYL |
| | | | RLVRQVLRHDGYFVTYFHPWEFYDLKSHPEFKMPFII |
| | | | KNHSGHELEQRLDRFIKAMKADKQEFITYVDFVNRQ |
| | | | KK |
| 59 | Fumarate_reductase_ | POAC47 | MAKNISFTIKYWKQNGPQDQGHFDTHEMKNIPDDTSF |
| | iron-sulfur_subunit | | LEMLDILNEELIAAGDEPFVFDHDCREGICGMCSLYIN |
| | | | GTPHGKTERGATTCQLYMRRFNDGDVITVEPWRSAG |
| | | | FPVIKDCMVDRTAFDKIIQAGGYTTIRTGQAQDANAIL |
| | | | ISKDNADEAMDCATCIGCGACVAACKNGSAMLFVSS |
| | | | KVSQLALLPQGKPEAAKRAKAMVAKMDEVGFGNCT |
| | | | NTRACEAVCPKNEKIANIARLNREFIKAKFAD |
| 60 | Serine/threonine- | P9WI71 | MSENKLSTNEQAQTADAPVKASYTEYKVIPSQGYCMI |
| | protein kinase_PknH | | VKCRKGDQTVVLKTLKEEYRERVLLRNALKREFKQC |
| | | | QRLNHSGIVRYQGLVEVDGYGLCIEEEYVEGRTLQAY |
| | | | LKENHTDDEKIAIINQIADALRYAHQQGVIHRNLKPSN |
| | | | VLVTTQGDYVKLIDFSVLSPEDVKPTAETTRFMAPEM |
| | | | KDETLTADATADIYSLGTIMKVMGLTLAYSEVIKRCC |
| | | | AFKRSDRYSNVDELLADLNNEGSSFSMPKIGKGTVVL |
| | | | GLIIAVVIGIGALLYNYGGALIDQVGKIDVSSVFSSDAE |
| | | | TAPEDTVKVNTAEQSDSLSTEAEAPAIGKLAFMNRMK |
| | | | PALYKDLDNIFEKNSADKAKLTKAIKTYYRGLIQAND |
| | | | TLDNEQRAEVDRVFGDYVKQKKAALN |
| 61 | Carboxy- | O34666 | MRKYICLLLFYLFTFLPLSAQQGNDSPLRKLQLAEMAI |
| | terminal_processing_ | | KNFYVDSVNEQKLVEDGIRGMLEKLDPHSTYTDAKE |
| | protease_CtpA | | TKAMNEPLQGDFEGIGVQFNMIEDTLVVIQPVVNGPS |
| | | | QKVGILAGDRIVSVNDSTIAGVKMARIDIMKMLRGKK |
| | | | GTKVKLGVVRRGVKGVLTFVVTRAKIPVHTINASYMI |
| | | | RPNVGYIRIESFGMKTHDEFMSAVDSLKKKGMKTLLL |
| | | | DLQDNGGGYLQSAVQISNEFLKNNDMIVYTEGRRAR |
| | | | RQNFKAIGNGRLQDVKVYVLVNELSASAAEIVTGAIQ |
| | | | DNDRGTVVGRRTFGKGLVQRPFDLPDGSMIRLTIAHY |
| | | | YTPSGRCIQKPYTKGDLKDYEMDIEKRFKHGELTNPD |
| | | | SIQFSDSLKYYTIRKHRVVYGGGGIMPDNFVPLDTTKF |
| | | | TRYHRMLAAKSIIINAYLKYADANRQALKAQYSSFDA |
| | | | FNKGYVVPQSLLDEIVAEGKKEKIEPKDAAELKATLP |
| | | | NIALQIKALTARDIWDMNEYFRVWNTQSDIVNKAVA |
| | | | LATGK |
| 62 | Cluster: | D9RRG3 | MKLTEQRSSMLHGVLLITLFACAAFYIGDMGWVKAL |
| | Uncharacterized | | SLSPMVVGIILGMLYANSLRNNLPDTWVPGIAFCGKR |
| | protein | | VLRFGIILYGFRLTFQDVVAVGFPAIIVDAIIVSGTILLG |
| | | | VLVGRLLKMDRSIALLTACGSGICGAAAVLGVDGAIR |
| | | | PKPYKTAVAVATVVIFGTLSMFLYPILYRAGIFDLSPD |
| | | | AMGIFAGSTIHEVAHVVGAGNAMGAAVSNSAIIVKMI |
| | | | RVMMLVPVLLVIAFFVAKNVAERDDEAGGSRKINIPW |
| | | | FAILFLVVIGFNSLNLLPKELVDFINTLDTFLLTMAMSA |
| | | | LGAETSIDKFKKAGFKPFLLAAILWCWLIGGGYCLAK |
| | | | YLVPVLGVAC |
| 63 | Cluster: Cna protein | X6Q2J4 | MNKQFLLAALWLSPLGLYAHKANGIGAVTWKNEAP |
| | B-type domain | | KERMIRGIDEDKTHQRFTLSGYVKDRNGEPLINATIYD |
| | protein | | LTTRQGTMTNAYGHFSLTLGEGQHEIRCSYVGYKTLI |
| | | | ETIDLSANQNHDIILQNEAQLDEVVVTTDLNSPLLKTQ |
| | | | TGKLSLSQKDIKTEYALLSSPDVIKTLQRTSGVADGME |
| | | | LASGLYVHGGNGDENLFLLDGTPLYHTNHSLGLFSSF |
| | | | NADVVKNVDFYKSGFPARYGGRLSSVIDVRTADGDL |
| | | | YKTHGSYRIGLLDGAFHIGGPIRKGKTSYNFGLRRSW |
| | | | MDLLTRPAFAIMNHKSDNEDKLSMSYFFHDLNFKLTN |
| | | | IFNERSRMSLSVYSGEDRLDAKDEWHSNNSSGYNDV |
| | | | DIYVNRFHWGNFNAALDWNYQFSPKLFANFTAVYTH |
| | | | NRSTVSSSDEWRFTRPGEKEQLTLTSHGYRSSIDDIGY |
| | | | RAAFDFRPSPRHHIRFGQDYTYHRFQPQTYNRFDNYQ |
| | | | TNSEAKADTIATHSYNKNVAHQLTFYAEDEMTLNEK |
| | | | WSLNGGVNADVFHISGKTFATLSPRLSMKFQPTERLS |
| | | | LKASYTLMSQFVHKIANSFLDLPTDYWVPTTARLHPM |
| | | | RSWQVAAGAYMKPNKHWLLSLEAYYKRSSHILQYSS |
| | | | WAGLEPPAANWDYMVMEGDGRSYGVELDADYNVS |
| | | | NLTLHGSYTLSWTQKKFDDFYDGWYYDKFDNRHKL |
| | | | TLTGRWNITKKIAAFAAWTFRTGNRMTIPTQYIGLPD |
| | | | VPAQEQGGLTFNSSDDNTLNFAYEKPNNVILPAYHRL |
| | | | DIGFDFHHTTKKGHERIWNLSFYNAYCHLNSLWVRV |
| | | | KIDSNNQMKIRNIAFIPVIPSFSYTFKF |
| 64 | Poly-beta-1,6-N- | P75905 | MSKQVFQTDSRQRWSYFKWTLRVILTILSLLGIVFLA |
| | acetyl-D- | | MFALEGSPQMPFRHDYRNAVTAASPYTKDNKTAKLY |
| | glucosamine_synthase | | KSFRDFFKEKKMHNNYAKATIKKQRFIGKADSVTQK |
| | | | YFREWDDPRIGVRSAWYVNWDKHAYISLKNNIKHLN |
| | | | MVLPEWFFINPKTDKVEYRIDKQALRLMRRTGIPVLP |
| | | | MLTNNYNSDFHPEAIGRIMRDEKKRMALINEMVRTC |
| | | | RHYGFAGINLDLEELNIQDNDLLVELLKDFSRVFHAN |
| | | | GLYVTQAVAPFNEDYNMQELAKYNDYLFLMAYDEH |
| | | | NIESQPGAVSSQRWVEKATDWAAKNVPNDKIVLGMA |
| | | | TYGYDWANGEGGTTVSFDQTMAIAQDADAKVKFDD |
| | | | DTYNVNFSYQNTDDGKIHHVFFTDAATTFNIMRFGAE |
| | | | YHLAGYGLWRLGTEDKRIWRFYGKDMSWENVARMS |
| | | | VAKLMQLNGTDDVNFVGSGEVLEVTTEPHPGDISIRID |
| | | | KDNRLISEEYYRALPSTYTIQRLGKCKDKQLVITFDDG |
| | | | PDSRWTPTVLSTLKKYNVPAAFFMVGLQMEKNLPLV |
| | | | KQVYEDGHTIGNHTFTHHNMIENSDRRSYAELKLTR |
| | | | MLIESVTGHSTILFRAPYNADADPTEHEEIWPMIVASR |
| | | | RNYLFVGESIDPNDWEPNVTSDQIYQRVIDGVHHEDG |
| | | | HIILLHDAGGSSRKPTLDALPRIIETLQHEGYQFISLEQ |
| | | | YLGMGKQTLMPEINKGKAYYAMQTNLWLAEMIYHV |
| | | | SDFLTALFLVFLALGMMRLIFMYVLMIREKRAENRRN |
| | | | YAPIDAATAPAVSIIVPGYNEEVNIVRTITTLKQQDYPN |
| | | | LHIYFVDDGSKDHTLERVHEAFDNDDTVTILAKKNGG |
| | | | KASALNYGIAACRSEYVVCIDADTQLKNDAVSRLMK |
| | | | HFIADTEKRVGAVAGNVKVGNQRNMLTYWQAIEYTS |
| | | | SQNFDRMAYSNINAITVVPGAIGAFRKEVIEAVGGFTT |
| | | | DTLAEDCDLTMSINEHGYIIENENYAVALTEAPETLRQ |
| | | | FVKQRIRWCFGVMQAFWKHRSSLFAPSKKGFGLWA |
| | | | MPNMLIFQYIIPTFSPLADVLMLIGLFTGNALQIFFYYLI |
| | | | FLVIDASVSIMAYIFEGERLWVLLWVIPQRFFYRWIMY |
| | | | YVLFKSYLKAIKGELQTWGVLKRTGHVKG |
| 65 | Cell_division_ | O34876 | MAKKRNKARSRHSLQVVTLCISTAMVLMLIGIVVLTG |
| | protein_FtsX | | FTSRNLSSYVKENLTITMILQPDMNTEESAALCERIRTL |
| | | | HYINSLNFISKEQALKDGTKELGANPAEFAGENPFTGE |
| | | | IEVQLKANYANNDSIRNIVQQLRTYRGVSDITYPQSLV |
| | | | ESVNQTLGKISLVLLVIAVLLTIISFSLINNTIRLSIYAHR |
| | | | FSIHTMKLVGGSWSFIRAPFLRRAVLEGLVSALLAIAV |
| | | | LGIGICLLYEKEPEITKLLSWDALIITAIVMLAFGVIIAT |
| | | | FCAWLSVNKFLRMKAGDLYKI |
| 66 | UDP-2,3- | P44046 | MKNIYFLSDAHLGSLAIDHRRTHERRLVRFLDSIKHKA |
| | diacylglucosamine | | AAVYLLGDMFDFWNEYKYVVPKGFTRFLGKISELTD |
| | hydrolase | | MGVEVHFFTGNHDLWTYGYLEKECGVILHRKPITTEI |
| | | | YDKVFYLAHGDGLGDPDPMFRFLRKVFHNRFCQRLL |
| | | | NFFHPWWGMQLGLNWAKRSRLKRKDGKEVPYLGED |
| | | | KEYLVQYTKEYMSTHKDIDYYIYGHRHIELDLTLSRK |
| | | | ARLLILGDWIWQFTYAVFDGEHMFLEEYVEGESKP |
| 67 | Poly-beta-1,6-N- | P75905 | MVGLDVLCYFIHAKGREKECYFERIIYQITCHSRTKCY |
| | acetyl-D- | | LCNIMKYSIIVPVFNRPDEVEELLESLLSQEEKDFEVVI |
| | glucosamine_synthase | | VEDGSQIPCKEVCDKYADKLDLHYYSKENSGPGQSR |
| | | | NYGAERAKGEYLLILDSDVVLPKGYICAVSEELKREP |
| | | | ADAFGGPDCAHESFTDTQKAISYSMTSFFTTGGIRGGK |
| | | | KKLDKFYPRSFNMGIRRDVYQELGGFSKMRFGEDIDF |
| | | | SIRIFKAGKRCRLFPEAWVWHKRRTDFRKFWKQVYN |
| | | | SGIARINLYKKYPESLKLVHLLPMVFTVGTALLVLMIL |
| | | | FGLFLQLFPIINVFGSVFIMMGLMPLVLYSVIICVDSTM |
| | | | QNNSLNIGLLSIEAAFIQLTGYGCGFISAWWKRCVCG |
| | | | MDEFAAYEKNFYK |
| 68 | Enolase | Q8DTS9 | MKIEKVHAREIMDSRGNPTVEVEVTLENGVMGRASV |
| | | | PSGASTGENEALELRDGDKNRFLGKGVLKAVENVNN |
| | | | LIAPALKGDCVLNQRAIDYKMLELDGTPTKSKLGANA |
| | | | ILGVSLAVAQAAAKALNIPLYRYIGGANTYVLPVPMM |
| | | | NIINGGAHSDAPIAFQEFMIRPVGAPSEKEGIRMGAEV |
| | | | FHALAKLLKKRGLSTAVGDEGGFAPKFDGIEDALDSII |
| | | | QAIKDAGYEPGKDVKIAMDCAASEFAVCEDGKWFYD |
| | | | YRQLKNGMPKDPNGKKLSADEQIAYLEHLITKYPIDSI |
| | | | EDGLDENDWENWVKLTSAIGDRCQLVGDDLFVTNV |
| | | | KFLEKGIKMGAANSILIKVNQIGSLTETLEAIEMAHRH |
| | | | GYTTVTSHRSGETEDTTIADIAVATNSGQIKTGSMSRT |
| | | | DRMAKYNQLIRIEEELGACAKYGYAKLK |
| 69 | Outer_membrane_ | Q8GOY6 | MKKLFTIAMLLGVTLGIHAQEVYSLQKCRELALQNNR |
| | efflux_protein_BepC | | QLKVSRMTVDVAENTRKAAKTKYLPRVDALAGYQH |
| | | | FSREISLLSDDQKNAFSNLGTNTFGQLGGQIGQNLTSL |
| | | | AQQGILSPQMAQQLGQLFSNVATPLTQVGNNIGQSIN |
| | | | DAFRSNTKNVYAGGIVVNQPIYMGGAIKAANDMAAI |
| | | | GEQVAQNNISLKRQLVLYGVDNAYWLAISLKKKEAL |
| | | | AIRYRDLAQKLNEDVKKMIREGVATRADGLKVEVAV |
| | | | NTADMQIARIQSGVSLAKMALCELCGLELNGDIPLSD |
| | | | EGDADLPPTPSTQFDNYTVSSSDTTGLNEARPELRLLQ |
| | | | NAVDLSIQNTKLIRSLYMPHVLLTAGYSVSNPNLFNGF |
| | | | QKRFTDLWNIGITVQVPVWNWGENKYKVRASKTATT |
| | | | IAQLEMDDVRKKIDLEIEQNRLRLKDANKQLATSQKN |
| | | | MAAAEENLRCANVGFKEGVMTVTEVMAAQTAWQTS |
| | | | RMAIIDAEISVKLAQTGLQKALGGL |
| 70 | Phosphoethanolamine_ | Q7CPC0 | MKRTFVTKMVKPIEENSLFFMFMLLVGAFTNVSHRN |
| | transferase_CptA | | VFGYIELIADVYIICFLLSLCQRTIRQGLVIMLSSVIYVV |
| | | | AIIDTCCKTLFDTPITPTMLLLAQETTGREATEFFLQYL |
| | | | NLKLFFSAADIILFLAFCHIVMAVKKMKFSTSYLKQPF |
| | | | VAFVLMFTIFVGMALSIYDKVQLYTVKNLSGLEVAVT |
| | | | NGFAHLYHPVERIVYGLYSNHLIAKQVDGVIMANQQI |
| | | | KVDSCSFTSPTIVLVIGESANRHHSQLYGYPLPTTPYQ |
| | | | LAMKNGKDSLAVFTNVVSPWNLTSKVFKQIFSLQSVD |
| | | | EKGDWSKYVLFPAVFKKAGYHVSFLSNQFPYGINYTP |
| | | | DWTNNLVGGFFLNHPQLNKQMFDYRNVTIHNYDEDL |
| | | | LNDYKEIISYKKPQLIIFHLLGQHFQYSLRCKSNMKKF |
| | | | GIKDYKRMDLTDKEKQTIADYDNATLYNDFVLNKIV |
| | | | EQFRNKDAIIVYLSDHGEDCYGKDVNMAGRLTEVEQI |
| | | | NLKKYHEEFEIPFWIWCSPIYKQRHRKIFTETLMARNN |
| | | | KFMTDDLPHLLLYLAGIKTKDYCEERNVISPSFNNNR |
| | | | RRLVLKTIDYDKALYQ |
| 71 | Dipeptide_and_ | P36837 | MFKNHPKGLLQAAFSNMGERFGYYIMNAVLALFLCS |
| | tripeptide_permease_B | | KFGLSDETSGLIASLFLAAIYVMSLVGGVIADRTQNYQ |
| | | | RTIESGLVVMALGYVALSIPVLATPENNSYLLAFTIFA |
| | | | LVLIAVGNGLFKGNLQAIVGQMYDDFETEAAKVSPER |
| | | | LKWAQGQRDAGFQIFYVFINLGALAAPFIAPVLRSWW |
| | | | LGRNGLTYDAALPQLCHKYINGTIGDNLGNLQELATK |
| | | | VGGNSADLASFCPHYLDVFNTGVHYSFIASVVTMLIS |
| | | | LIIFMSSKKLFPMPGKKEQIVNVEYTDEEKASMAKEIK |
| | | | QRMYALFAVLGISVFFWFSFHQNGQSLSFFARDFVNT |
| | | | DSVAPEIWQAVNPFFVISLTPLIMWVFAYFTKKGKPIS |
| | | | TPRKIAYGMGIAGFAYLFLMGFSLVHNYPSAEQFTSLE |
| | | | PAVRATMKAGPMILILTYFFLTVAELFISPLGLSFVSKV |
| | | | APKNLQGLCQGLWLGATAVGNGFLWIGPLMYNKWSI |
| | | | WTCWLVFAIVCFISMVVMFGMVKWLERVTKS |
| 72 | C4- | Q9I4F5 | MQKKIKIGLLPRVIIAILLGLFLGYYLPDPAVRVFLTFN |
| | dicarboxylate_ | | SIFSQFLGFMIPLIIIGLVTPAIAGIGKGAGKLLLATVAI |
| | transport_protein_2 | | AYVDTIVAGGLSYGTGTWLFPSMIASTGGAIPHIDKAT |
| | | | ELTPYFTINIPAMVDVMSSLVFSFIAGLGIAYGGLRTM |
| | | | ENLFNEFKTVIEKVIEKAIIPLLPLYIFGVFLSMTHNGQ |
| | | | ARQVLLVFSQIIIVILVLHVLILIYEFCIAGAIVKHNPFR |
| | | | LLWNMLPAYLTALGTSSSAATIPVTLKQTVKNGVSEE |
| | | | VAGFVVPLCATIHLSGSAMKITACALTICMLTDLPHDP |
| | | | GLFIYFILMLAIIMVAAPGVPGGAIMAALAPLSSILGEN |
| | | | EEAQALMIALYIAMDSFGTACNVTGDGAIALAVNKFF |
| | | | GKKKETSILS |
| 73 | Inner_membrane_ | P76090 | MISVYSIKPQFQRVLTPILELLHRAKVTANQITLWACV |
| | protein_YnbA | | LSLVIGILFWFAGDVGTWLYLCLPVGLLIRMALNALD |
| | | | GMMARRYNQITRKGELLNEVGDVVSDTIIYFPLLKYH |
| | | | PESLYFIVAFIALSIINEYAGVMGKVLSAERRYDGPMG |
| | | | KSDRAFVLGLYGVVCLFGINLSGYSVYIFGVIDLLLVL |
| | | | STWIRIKKTLKVTRNSQTPE |
| 74 | 2′,3′-cyclic- | P08331 | MKLSTILLSIMLGLSSSTMAQQKDVTIKLIETTDVHGS |
| | nucleotide | | FFPYDFITRKPKSGSMARVYTLVEELRKKDGKDNVYL |
| | | | LDNGDILQGQPISYYYNYVAPEKTNIAASVLNYMGYD |
| | | | VATVGNHDIETGHKVYDKWFKELKFPILGANIIDTKT |
| | | | NKPYILPYYTIKKKNGIKVCVIGMLTPAIPNWLKESIW |
| | | | SGLRFEEMVSCAKRTMAEVKTQEKPDVIVGLFHSGW |
| | | | DGGIKTPEYDEDASKKVAKEVPGFDIVFFGHDHTPHS |
| | | | SIEKNIVGKDVICLDPANNAQRVAIATLTLRPKTVKGK |
| | | | RQYTVTKATGELVDVKELKADDAFIQHFQPEIDAVKA |
| | | | WSDQVIGRFENTIYSKDSYFGNSAFNDLILNLELEITK |
| | | | ADIAFNAPLLFNASIKAGPITVADMFNLYKYENNLCT |
| | | | MRLTGKEIRKHLEMSYDLWCNTMKSPEDHLLLLSST |
| | | | QNDAQRLGFKNFSFNFDSAAGIDYEVDVTKPDGQKV |
| | | | RILRMSNGEPFDENKWYTVAVNSYRANGGGELLTKG |
| | | | AGIPRDSLKSRIIWESPKDQRHYLMEEIKKAGVMNPQP |
| | | | NHNWKFIPETWTVPAAARDRKLLFGE |
| 75 | Fe(2+)_transporter | P33650 | MKLSELKTGETGVIVKVSGHGGFRKRIIEMGFIKGKTV |
| | FeoB | | EVLLNAPLQDPVKYKIMGYEVSLRHSEADQIEVLSDV |
| | | | KTHSVGNEEEQEDNQLEMDSTTYDSTDKELTPEKQSD |
| | | | AVRRKNHTINVALVGNPNCGKTSLFNFASGAHERVG |
| | | | NYSGVTVDAKVGRAEFDGYVENLVDLPGTYSLSAYS |
| | | | PEELYVRKQLVDKTPDVVINVIDSSNLERNLYLTTQLI |
| | | | DMHIRMVCALNMFDETEQRGDHIDAQKLSELFGVPM |
| | | | IPTVFTNGRGVKELFRQIIAVYEGKEDESLQFRHIHINH |
| | | | GHEIENGIKEMQEHLKKYPELCHRYSTRYLAIKLLEH |
| | | | DKDVEQLVSPLGDSIEIFNHRDTAAARVKEETGNDSE |
| | | | TAIMDAKYGFINGALKEANFSTGDKKDTYQTTHVIDH |
| | | | VLTNKYFGFPIFFLVLLVMFTATFVIGQYPMDWIEAG |
| | | | VGWLGEFISKNMPAGPVKDMIVDGIIGGVGAVIVFLP |
| | | | QILILYFFISYMEDCGYMSRAAFIMDRLMHKMGLHGK |
| | | | SFIPLIMGFGCNVPAVMATRTIESRRSRLITMLILPLMS |
| | | | CSARLPIYVMITGSFFALKYRSLAMLSLYIIGVLMAVA |
| | | | MSRLFSAFVVKGEDTPFVMELPPYRFPTWKAIGRHTW |
| | | | EKGKQYLKKMGGIILVASIIVWALGYFPLPDDPNMDN |
| | | | QARQEQSYIGRIGKAVEPVFRPQGFNWKLDVGLLSG |
| | | | MGAKEIVASTMGVLYSNDGSFSDDNGYSSETGKYSK |
| | | | LHNLITKDVATMHHISYEEAEPIATLTAFSFLLFVLLYF |
| | | | PCVATIAAIKGETGSWGWALFAAGYTTALAWIVSAV |
| | | | VFQVGMLFM |
| 76 | UDP-N- | P9WJMI | MESFIIEGGHQLSGTIAPQGAKNEALEVICATLLTSEEV |
| | acetylglucosamine | | IIRNVPDILDVNNLIKLLQDIGVKVKKLAPNEFSFQADE |
| | | | VNLDYLESSDFVKKCSSLRGSVLMIGPLLGRFGKATIA |
| | | | KPGGDKIGRRRLDTHFLGFKNLGAHFGRVEDRDVYEI |
| | | | QADKLVGTYMLLDEASITGTANIIMAAVLAEGTTTIY |
| | | | NAACEPYIQQLCKMLNAMGAKISGIASNLITIEGVKEL |
| | | | HSADHRILPDMIEVGSFIGIAAMIGDGVRIKDVSVPNL |
| | | | GLILDTFHRLGVQIIVDNDDLIIPRQDHYVIDSFIDGTIM |
| | | | TISDAPWPGLTPDLISVLLVVATQAQGSVLFHQKMFES |
| | | | RLFFVDKLIDMGAQIILCDPHRAVVVGHDNAKKLRAG |
| | | | RMSSPDIRAGIALLIAALTAQGTSRIDNIVQIDRGYENI |
| | | | EGRLNALGAKIQRAEVC |
| 77 | Ribitol-5- | Q8RKI9 | MNIAVIFAGGSGLRMHTKSRPKQFLDLNGKPIIIYTLEL |
| | phosphate_cytidylyl | | FDNHPNIDAIVVACIESWIPFLEKQLRKFEINKVVKIIPG |
| | transferase | | GKSGQESIYKGLCAAEEYAQSKGVSNEETTVLIHDGV |
| | | | RPLITEETITDNIKKVEEVGSCITCIPATETLIVKQADDA |
| | | | LEIPSRADSFIARAPQSFRLIDIITAHRRSLAEGKADFID |
| | | | SCTMMSHYGYKLGTIIGPMENIKITTPTDFFVLRAMVK |
| | | | VHEDQQIFGL |

In some embodiments, the hemoglobin-dependent Prevotella strain is a strain of Prevotella bacteria comprising one or more of the proteins listed in Table 1 and that is free or substantially free of one or more proteins listed in Table 2. In some embodiments, the hemoglobin-dependent Prevotella strain is a strain of Prevotella bacteria that comprises all of the proteins listed in Table 1 and/or all of the genes encoding the proteins listed in Table 1 and that is free of all of the proteins listed in Table 2 and/or all of the genes encoding the proteins listed in Table 2.

Modified Bacteria and mEVs

In some embodiments, the bacteria of the pharmaceutical agent or from which the mEVs of the pharmaceutical agent are obtained are modified to reduce toxicity or other adverse effects, to enhance delivery) (e.g., oral delivery) (e.g., by improving acid resistance, muco-adherence and/or penetration and/or resistance to bile acids, digestive enzymes, resistance to anti-microbial peptides and/or antibody neutralization), to target desired cell types (e.g., M-cells, goblet cells, enterocytes, dendritic cells, macrophages), to enhance their immunomodulatory and/or therapeutic effect of the bacteria and/or mEVs (e.g., either alone or in combination with another therapeutic agent), and/or to enhance immune activation or suppression by the bacteria and/or mEVs (such as smEVs and/or pmEVs) (e.g., through modified production of polysaccharides, pili, fimbriae, adhesins). In some embodiments, the engineered bacteria described herein are modified to improve bacteria and/or mEV (such as smEV and/or pmEV) manufacturing (e.g., higher oxygen tolerance, stability, improved freeze-thaw tolerance, shorter generation times). For example, in some embodiments, the engineered bacteria described include bacteria harboring one or more genetic changes, such change being an insertion, deletion, translocation, or substitution, or any combination thereof, of one or more nucleotides contained on the bacterial chromosome or endogenous plasmid and/or one or more foreign plasmids, wherein the genetic change may result in the overexpression and/or underexpression of one or more genes. The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

In some aspects, the bacteria and/or mEVs (such as smEVs and/or pmEVs) described herein are modified such that they comprise, are linked to, and/or are bound by a therapeutic moiety.

In some embodiments, the therapeutic moiety is a cancer-specific moiety. In some embodiments, the cancer-specific moiety has binding specificity for a cancer cell (e.g., has binding specificity for a cancer-specific antigen). In some embodiments, the cancer-specific moiety comprises an antibody or antigen binding fragment thereof. In some embodiments, the cancer-specific moiety comprises a T cell receptor or a chimeric antigen receptor (CAR). In some embodiments, the cancer-specific moiety comprises a ligand for a receptor expressed on the surface of a cancer cell or a receptor-binding fragment thereof. In some embodiments, the cancer-specific moiety is a bipartite fusion protein that has two parts: a first part that binds to and/or is linked to the bacterium and a second part that is capable of binding to a cancer cell (e.g., by having binding specificity for a cancer-specific antigen). In some embodiments, the first part is a fragment of or a full-length peptidoglycan recognition protein, such as PGRP. In some embodiments the first part has binding specificity for the mEV (e.g., by having binding specificity for a bacterial antigen). In some embodiments, the first and/or second part comprises an antibody or antigen binding fragment thereof. In some embodiments, the first and/or second part comprises a T cell receptor or a chimeric antigen receptor (CAR). In some embodiments, the first and/or second part comprises a ligand for a receptor expressed on the surface of a cancer cell or a receptor-binding fragment thereof. In certain embodiments, co-administration of the cancer-specific moiety with the pharmaceutical agent (either in combination or in separate administrations) increases the targeting of the pharmaceutical agent to the cancer cells.

In some embodiments, the bacteria and/or mEVs described herein can be modified such that they comprise, are linked to, and/or are bound by a magnetic and/or paramagnetic moiety (e.g., a magnetic bead). In some embodiments, the magnetic and/or paramagnetic moiety is comprised by and/or directly linked to the bacteria. In some embodiments, the magnetic and/or paramagnetic moiety is linked to and/or a part of a bacteria- or an mEV-binding moiety that binds to the bacteria or mEV. In some embodiments, the bacteria- or mEV-binding moiety is a fragment of or a full-length peptidoglycan recognition protein, such as PGRP. In some embodiments the bacteria- or mEV-binding moiety has binding specificity for the bacteria or mEV (e.g., by having binding specificity for a bacterial antigen). In some embodiments, the bacteria- or mEV-binding moiety comprises an antibody or antigen binding fragment thereof. In some embodiments, the bacteria- or mEV-binding moiety comprises a T cell receptor or a chimeric antigen receptor (CAR). In some embodiments, the bacteria- or mEV-binding moiety comprises a ligand for a receptor expressed on the surface of a cancer cell or a receptor-binding fragment thereof. In certain embodiments, co-administration of the magnetic and/or paramagnetic moiety with the bacteria or mEVs (either together or in separate administrations) can be used to increase the targeting of the mEVs (e.g., to cancer cells and/or a part of a subject where cancer cells are present.

Production of Processed Microbial Extracellular Vesicles (pmEVs)

In certain aspects, the pmEVs described herein can be prepared using any method known in the art.

In some embodiments, the pmEVs are prepared without a pmEV purification step. For example, in some embodiments, bacteria from which the pmEVs described herein are released are killed using a method that leaves the bacterial pmEVs intact, and the resulting bacterial components, including the pmEVs, are used in the methods and compositions described herein. In some embodiments, the bacteria are killed using an antibiotic (e.g., using an antibiotic described herein). In some embodiments, the bacteria are killed using UV irradiation.

In some embodiments, the pmEVs described herein are purified from one or more other bacterial components. Methods for purifying pmEVs from bacteria (and optionally, other bacterial components) are known in the art. In some embodiments, pmEVs are prepared from bacterial cultures using methods described in Thein, et al. (J. Proteome Res. 9(12):6135-6147 (2010)) or Sandrini, et al. (Bio-protocol 4(21): e1287 (2014)), each of which is hereby incorporated by reference in its entirety. In some embodiments, the bacteria are cultured to high optical density and then centrifuged to pellet bacteria (e.g., at 10,000-15,000×g for 10-15 min at room temperature or 4° C.). In some embodiments, the supernatants are discarded and cell pellets are frozen at −80° C. In some embodiments, cell pellets are thawed on ice and resuspended in 100 mM Tris-HCl, pH 7.5 supplemented with 1 mg/mL DNase I. In some embodiments, cells are lysed using an Emulsiflex C-3 (Avestin, Inc.) under conditions recommended by the manufacturer. In some embodiments, debris and unlysed cells are pelleted by centrifugation at 10,000×g for 15 min at 4° C. In some embodiments, supernatants are then centrifuged at 120,000×g for 1 hour at 4° C. In some embodiments, pellets are resuspended in ice-cold 100 mM sodium carbonate, pH 11, incubated with agitation for 1 hr at 4° C., and then centrifuged at 120,000×g for 1 hour at 4° C. In some embodiments, pellets are resuspended in 100 mM Tris-HCl, pH 7.5, re-centrifuged at 120,000×g for 20 min at 4° C., and then resuspended in 0.1 M Tris-HCl, pH 7.5 or in PBS. In some embodiments, samples are stored at −20° C.

In certain aspects, pmEVs are obtained by methods adapted from Sandrini et al, 2014. In some embodiments, bacterial cultures are centrifuged at 10,000-15,500×g for min at room temp or at 4° C. In some embodiments, cell pellets are frozen at −80° C. and supernatants are discarded. In some embodiments, cell pellets are thawed on ice and resuspended in 10 mM Tris-HCl, pH 8.0, 1 mM EDTA supplemented with 0.1 mg/mL lysozyme. In some embodiments, samples are incubated with mixing at room temp or at 37° C. for 30 min. In some embodiments, samples are re-frozen at −80° C. and thawed again on ice. In some embodiments, DNase I is added to a final concentration of 1.6 mg/mL and MgCl2 to a final concentration of 100 mM. In some embodiments, samples are sonicated using a QSonica Q500 sonicator with 7 cycles of 30 sec on and 30 sec off. In some embodiments, debris and unlysed cells are pelleted by centrifugation at 10,000×g for 15 min. at 4° C. In some embodiments, supernatants are then centrifuged at 110,000×g for 15 min at 4° C. In some embodiments, pellets are resuspended in 10 mM Tris-HCl, pH 8.0, 2% Triton X-100 and incubated 30-60 min with mixing at room temperature. In some embodiments, samples are centrifuged at 110,000×g for 15 min at 4° C. In some embodiments, pellets are resuspended in PBS and stored at −20° C.

In certain aspects, a method of forming (e.g., preparing) isolated bacterial pmEVs, described herein, comprises the steps of: (a) centrifuging a bacterial culture, thereby forming a first pellet and a first supernatant, wherein the first pellet comprises cells; (b) discarding the first supernatant; (c) resuspending the first pellet in a solution; (d) lysing the cells; (e) centrifuging the lysed cells, thereby forming a second pellet and a second supernatant; (f) discarding the second pellet and centrifuging the second supernatant, thereby forming a third pellet and a third supernatant; (g) discarding the third supernatant and resuspending the third pellet in a second solution, thereby forming the isolated bacterial pmEVs.

In some embodiments, the method further comprises the steps of: (h) centrifuging the solution of step (g), thereby forming a fourth pellet and a fourth supernatant; (i) discarding the fourth supernatant and resuspending the fourth pellet in a third solution. In some embodiments, the method further comprises the steps of: (j) centrifuging the solution of step (i), thereby forming a fifth pellet and a fifth supernatant; and (k) discarding the fifth supernatant and resuspending the fifth pellet in a fourth solution.

In some embodiments, the centrifugation of step (a) is at 10,000×g. In some embodiments the centrifugation of step (a) is for 10-15 minutes. In some embodiments, the centrifugation of step (a) is at 4° C. or room temperature. In some embodiments, step (b) further comprises freezing the first pellet at −80° C. In some embodiments, the solution in step (c) is 100 mM Tris-HCl, pH 7.5 supplemented with 1 mg/ml DNaseI. In some embodiments, the solution in step (c) is 10 mM Tris-HCl, pH 8.0, 1 mM EDTA, supplemented with 0.1 mg/ml lysozyme. In some embodiments, step (c) further comprises incubating for 30 minutes at 37° C. or room temperature. In some embodiments, step (c) further comprises freezing the first pellet at −80° C. In some embodiments, step (c) further comprises adding DNase I to a final concentration of 1.6 mg/ml. In some embodiments, step (c) further comprises adding MgCl₂ to a final concentration of 100 mM. In some embodiments, the cells are lysed in step (d) via homogenization. In some embodiments, the cells are lysed in step (d) via emulsiflex C3. In some embodiments, the cells are lysed in step (d) via sonication. In some embodiments, the cells are sonicated in 7 cycles, wherein each cycle comprises 30 seconds of sonication and 30 seconds without sonication. In some embodiments, the centrifugation of step (e) is at 10,000×g. In some embodiments, the centrifugation of step (e) is for 15 minutes. In some embodiments, the centrifugation of step (e) is at 4° C. or room temperature.

In some embodiments, the centrifugation of step (f) is at 120,000×g. In some embodiments, the centrifugation of step (f) is at 110,000×g. In some embodiments, the centrifugation of step (f) is for 1 hour. In some embodiments, the centrifugation of step (f) is for 15 minutes. In some embodiments, the centrifugation of step (f) is at 4° C. or room temperature. In some embodiments, the second solution in step (g) is 100 mM sodium carbonate, pH 11. In some embodiments, the second solution in step (g) is 10 mM Tris-HCl pH 8.0, 2% triton X-100. In some embodiments, step (g) further comprises incubating the solution for 1 hour at 4° C. In some embodiments, step (g) further comprises incubating the solution for 30-60 minutes at room temperature. In some embodiments, the centrifugation of step (h) is at 120,000×g. In some embodiments, the centrifugation of step (h) is at 110,000×g. In some embodiments, the centrifugation of step (h) is for 1 hour. In some embodiments, the centrifugation of step (h) is for 15 minutes. In some embodiments, the centrifugation of step (h) is at 4° C. or room temperature. In some embodiments, the third solution in step (i) is 100 mM Tris-HCl, pH 7.5. In some embodiments, the third solution in step (i) is PBS. In some embodiments, the centrifugation of step (j) is at 120,000×g. In some embodiments, the centrifugation of step (j) is for 20 minutes. In some embodiments, the centrifugation of step (j) is at 4° C. or room temperature. In some embodiments, the fourth solution in step (k) is 100 mM Tris-HCl, pH 7.5 or PBS.

pmEVs obtained by methods provided herein may be further purified by size based column chromatography, by affinity chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in 35% Optiprep in PBS. In some embodiments, if filtration was used to concentrate the filtered supernatant, the concentrate is diluted using 60% Optiprep to a final concentration of 35% Optiprep. Samples are applied to a 35-60% discontinuous sucrose gradient and centrifuged at 200,000×g for 3-24 hours at 4° C.

In some embodiments, to confirm sterility and isolation of the pmEV preparations, pmEVs are serially diluted onto agar medium used for routine culture of the bacteria being tested, and incubated using routine conditions. Non-sterile preparations are passed through a 0.22 um filter to exclude intact cells. To further increase purity, isolated pmEVs may be DNase or proteinase K treated.

In some embodiments, the sterility of the pmEV preparations can be confirmed by plating a portion of the pmEVs onto agar medium used for standard culture of the bacteria used in the generation of the pmEVs and incubating using standard conditions.

In some embodiments select pmEVs are isolated and enriched by chromatography and binding surface moieties on pmEVs. In other embodiments, select pmEVs are isolated and/or enriched by fluorescent cell sorting by methods using affinity reagents, chemical dyes, recombinant proteins or other methods known to one skilled in the art.

The pmEVs can be analyzed, e.g., as described in Jeppesen, et al. Cell 177:428 (2019).

In some embodiments, pmEVs are lyophilized.

In some embodiments, pmEVs are gamma irradiated (e.g., at 17.5 or 25 kGy).

In some embodiments, pmEVs are UV irradiated.

In some embodiments, pmEVs are heat inactivated (e.g., at 50° C. for two hours or at 90° C. for two hours).

In some embodiments, pmEVs are acid treated.

In some embodiments, pmEVs are oxygen sparged (e.g., at 0.1 vvm for two hours).

The phase of growth can affect the amount or properties of bacteria. In the methods of pmEV preparation provided herein, pmEVs can be isolated, e.g., from a culture, at the start of the log phase of growth, midway through the log phase, and/or once stationary phase growth has been reached.

Production of Secreted Microbial Extracellular Vesicles (smEVs)

In certain aspects, the smEVs described herein can be prepared using any method known in the art.

In some embodiments, the smEVs are prepared without an smEV purification step. For example, in some embodiments, bacteria described herein are killed using a method that leaves the smEVs intact and the resulting bacterial components, including the smEVs, are used in the methods and compositions described herein. In some embodiments, the bacteria are killed using an antibiotic (e.g., using an antibiotic described herein). In some embodiments, the bacteria are killed using UV irradiation. In some embodiments, the bacteria are heat-killed.

In some embodiments, the smEVs described herein are purified from one or more other bacterial components. Methods for purifying smEVs from bacteria are known in the art. In some embodiments, smEVs are prepared from bacterial cultures using methods described in S. Bin Park, et al. PLoS ONE. 6(3):e17629 (2011) or G. Norheim, et al. PLoS ONE. 10(9): e0134353 (2015) or Jeppesen, et al. Cell 177:428 (2019), each of which is hereby incorporated by reference in its entirety. In some embodiments, the bacteria are cultured to high optical density and then centrifuged to pellet bacteria (e.g., at 10,000×g for 30 min at 4° C., at 15,500×g for 15 min at 4° C.). In some embodiments, the culture supernatants are then passed through filters to exclude intact bacterial cells (e.g., a 0.22 μm filter). In some embodiments, the supernatants are then subjected to tangential flow filtration, during which the supernatant is concentrated, species smaller than 100 kDa are removed, and the media is partially exchanged with PBS. In some embodiments, filtered supernatants are centrifuged to pellet bacterial smEVs (e.g., at 100,000-150,000×g for 1-3 hours at 4° C., at 200,000×g for 1-3 hours at 4° C.). In some embodiments, the smEVs are further purified by resuspending the resulting smEV pellets (e.g., in PBS), and applying the resuspended smEVs to an Optiprep (iodixanol) gradient or gradient (e.g., a 30-60% discontinuous gradient, a 0-45% discontinuous gradient), followed by centrifugation (e.g., at 200,000×g for 4-20 hours at 4° C.). smEV bands can be collected, diluted with PBS, and centrifuged to pellet the smEVs (e.g., at 150,000×g for 3 hours at 4° C., at 200,000×g for 1 hour at 4° C.). The purified smEVs can be stored, for example, at −80° C. or −20° C. until use. In some embodiments, the smEVs are further purified by treatment with DNase and/or proteinase K.

For example, in some embodiments, cultures of bacteria can be centrifuged at 11,000×g for 20-40 min at 4° C. to pellet bacteria. Culture supernatants may be passed through a 0.22 μm filter to exclude intact bacterial cells. Filtered supernatants may then be concentrated using methods that may include, but are not limited to, ammonium sulfate precipitation, ultracentrifugation, or filtration. For example, for ammonium sulfate precipitation, 1.5-3 M ammonium sulfate can be added to filtered supernatant slowly, while stirring at 4° C. Precipitations can be incubated at 4° C. for 8-48 hours and then centrifuged at 11,000×g for 20-40 min at 4° C. The resulting pellets contain bacteria smEVs and other debris. Using ultracentrifugation, filtered supernatants can be centrifuged at 100,000-200,000×g for 1-16 hours at 4° C. The pellet of this centrifugation contains bacteria smEVs and other debris such as large protein complexes. In some embodiments, using a filtration technique, such as through the use of an Amicon Ultra spin filter or by tangential flow filtration, supernatants can be filtered so as to retain species of molecular weight >50 or 100 kDa.

Alternatively, smEVs can be obtained from bacteria cultures continuously during growth, or at selected time points during growth, for example, by connecting a bioreactor to an alternating tangential flow (ATF) system (e.g., XCell ATF from Repligen). The ATF system retains intact cells (>0.22 um) in the bioreactor, and allows smaller components (e.g., smEVs, free proteins) to pass through a filter for collection. For example, the system may be configured so that the <0.22 um filtrate is then passed through a second filter of 100 kDa, allowing species such as smEVs between 0.22 um and 100 kDa to be collected, and species smaller than 100 kDa to be pumped back into the bioreactor. Alternatively, the system may be configured to allow for medium in the bioreactor to be replenished and/or modified during growth of the culture. smEVs collected by this method may be further purified and/or concentrated by ultracentrifugation or filtration as described above for filtered supernatants.

smEVs obtained by methods provided herein may be further purified by size-based column chromatography, by affinity chromatography, by ion-exchange chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in PBS and 3 volumes of 60% Optiprep are added to the sample. In some embodiments, if filtration was used to concentrate the filtered supernatant, the concentrate is diluted using 60% Optiprep to a final concentration of 35% Optiprep. Samples are applied to a 0-45% discontinuous Optiprep gradient and centrifuged at 200,000×g for 3-24 hours at 4° C., e.g., 4-24 hours at 4° C.

In some embodiments, to confirm sterility and isolation of the smEV preparations, smEVs are serially diluted onto agar medium used for routine culture of the bacteria being tested, and incubated using routine conditions. Non-sterile preparations are passed through a 0.22 um filter to exclude intact cells. To further increase purity, isolated smEVs may be DNase or proteinase K treated.

In some embodiments, for preparation of smEVs used for in vivo injections, purified smEVs are processed as described previously (G. Norheim, et al. PLoS ONE. 10(9): e0134353 (2015)). Briefly, after sucrose gradient centrifugation, bands containing smEVs are resuspended to a final concentration of 50 μg/mL in a solution containing 3% sucrose or other solution suitable for in vivo injection known to one skilled in the art. This solution may also contain adjuvant, for example aluminum hydroxide at a concentration of 0-0.5% (w/v). In some embodiments, for preparation of smEVs used for in vivo injections, smEVs in PBS are sterile-filtered to <0.22 um.

In certain embodiments, to make samples compatible with further testing (e.g., to remove sucrose prior to TEM imaging or in vitro assays), samples are buffer exchanged into PBS or 30 mM Tris, pH 8.0 using filtration (e.g., Amicon Ultra columns), dialysis, or ultracentrifugation (200,000×g, ≥3 hours, 4° C.) and resuspension.

In some embodiments, the sterility of the smEV preparations can be confirmed by plating a portion of the smEVs onto agar medium used for standard culture of the bacteria used in the generation of the smEVs and incubating using standard conditions.

In some embodiments, select smEVs are isolated and enriched by chromatography and binding surface moieties on smEVs. In other embodiments, select smEVs are isolated and/or enriched by fluorescent cell sorting by methods using affinity reagents, chemical dyes, recombinant proteins or other methods known to one skilled in the art.

The smEVs can be analyzed, e.g., as described in Jeppesen, et al. Cell 177:428 (2019).

In some embodiments, smEVs are lyophilized.

In some embodiments, smEVs are gamma irradiated (e.g., at 17.5 or 25 kGy).

In some embodiments, smEVs are UV irradiated.

In some embodiments, smEVs are heat inactivated (e.g., at 50° C. for two hours or at 90° C. for two hours).

In some embodiments, smEVs s are acid treated.

In some embodiments, smEVs are oxygen sparged (e.g., at 0.1 vvm for two hours).

The phase of growth can affect the amount or properties of bacteria and/or smEVs produced by bacteria. For example, in the methods of smEV preparation provided herein, smEVs can be isolated, e.g., from a culture, at the start of the log phase of growth, midway through the log phase, and/or once stationary phase growth has been reached.

The growth environment (e.g., culture conditions) can affect the amount of smEVs produced by bacteria. For example, the yield of smEVs can be increased by an smEV inducer, as provided in Table 3.

TABLE 3
Culture Techniques to Increase smEV Production

| smEV | | |
|---|---|---|
| inducement | smEV inducer | Acts on |
| Temperature | Heat | stress response |
| | RT to 37° C. temp change | simulates infection |
| | 37 to 40° C. temp change | febrile infection |
| ROS | Plumbagin | oxidative stress response |
| | Cumene hydroperoxide | oxidative stress response |
| | Hydrogen Peroxide | oxidative stress response |
| Antibiotics | Ciprofloxacin | bacterial SOS response |
| | Gentamycin | protein synthesis |
| | Polymyxin B | outer membrane |
| | D-cylcloserine | cell wall |
| Osmolyte | NaCl | osmotic stress |
| Metal Ion | Iron Chelation | iron levels |
| Stress | EDTA | removes divalent cations |
| | Low Hemin | iron levels |
| Media | Lactate | growth |
| additives or | Amino acid deprivation | stress |
| removal | Hexadecane | stress |
| | Glucose | growth |
| | Sodium bicarbonate | ToxT induction |
| | PQS | vesiculator (from bacteria) |
| | Diamines + DFMO | membrane anchoring |
| | | (negativicutes only) |
| | High nutrients | enhanced growth |
| | Low nutrients | |
| Other | Oxygen | oxygen stress in anaerobe |
| mechanisms | No Cysteine | oxygen stress in anaerobe |
| | Inducing biofilm or | |
| | floculation | |
| | Diauxic Growth | |
| | Phage | |
| | Urea | |

In the methods of smEVs preparation provided herein, the method can optionally include exposing a culture of bacteria to an smEV inducer prior to isolating smEVs from the bacterial culture. The culture of bacteria can be exposed to an smEV inducer at the start of the log phase of growth, midway through the log phase, and/or once stationary phase growth has been reached.

### Solid Dosage Forms

In certain embodiments, provided herein are solid dosage forms (e.g., pharmaceutical compositions having a solid dosage form) comprising a pharmaceutical agent that contains bacteria and/or mEVs (such as smEVs and/or pmEVs) and a spirulina component. In some embodiments, the pharmaceutical agent can optionally contain one or more additional components, such as a cryoprotectant. The pharmaceutical agent can be lyophilized (e.g., resulting in a powder). The pharmaceutical agent can be combined with one or more excipients (e.g., pharmaceutically acceptable excipients) in the solid dosage form.

In certain aspects provided herein are solid dosage forms of pharmaceutical compositions. In certain embodiments, the solid dosage form comprises a pharmaceutical agent (e.g., bacteria and/or an agent (e.g., component) of bacterial origin, such as mEVs, a powder comprising bacteria and/or an agent (e.g., component) of bacterial origin, such as mEVs) and one or more disintegration agents. In certain embodiments, the total pharmaceutical agent mass is at least 5%, 10%, 15%, 20% or 25% of the total mass of the pharmaceutical composition. In some embodiments the total pharmaceutical agent mass is no more than 45%, 40%, 35%, 30%, or 25% of the total mass of the pharmaceutical composition. In some embodiments, the total mass of the one or more disintegrating agents is at least 30%, at least 35%, at least 40%, at least 45%, or at least 50% of the total mass of the pharmaceutical composition. In some embodiments, the total mass of the one or more disintegrating agents is no more than 70%, 65%, 60%, or 55% of the total mass of the pharmaceutical composition. In some embodiments, the one or more disintegration agents comprise low-substituted hydroxypropyl cellulose (L-HPC), croscarmellose sodium (Ac-Di-Sol), and/or crospovidone (PVPP).

In certain embodiments, the solid dosage forms provided herein comprise L-HPC. In some embodiments, the L-HPC is (or comprises L-HPC) of grade LH-B1. In certain embodiments, the total L-HPC mass is at least 22%, 23%, 24%, 25%, 26%, 27%, 28%, 29%, 30%, 31%, 32%, 33%, 34%, 35%, 36%, 37%, 38%, 39%, 40%, 41%, or 42% of the total mass of the pharmaceutical composition. In certain embodiments, the total L-HPC mass is no more than 22%, 23%, 24%, 25%, 26%, 27%, 28%, 29%, 30%, 31%, 32%, 33%, 34%, 35%, 36%, 37%, 38%, 39%, 40%, 41%, or 42% of the total mass of the pharmaceutical composition. In certain embodiments, the total L-HPC mass is about 22%, 23%, 24%, 25%, 26%, 27%, 28%, 29%, 30%, 31%, 32%, 33%, 34%, 35%, 36%, 37%, 38%, 39%, 40%, 41%, or 42% of the total mass of the pharmaceutical composition.

In certain embodiments, the solid dosage forms provided herein comprise Ac-Do-Sol. In some embodiments, the Ac-Di-Sol is (or comprises Ac-Di-Sol) of grade SD-711. In certain embodiments, the total Ac-Di-Sol mass is at least 0.01%, 0.1%, 1%, 2%, 3%, 4%, 5%, 6%, 7%, 8%, 9%, 10%, 11%, 12%, 13%, 14%, 15%, or 16% of the total mass of the pharmaceutical composition. In certain embodiments, the total Ac-Di-Sol mass is no more than 1%, 2%, 3%, 4%, 5%, 6%, 7%, 8%, 9%, 10%, 11%, 12%, 13%, 14%, 15%, or 16% of the total mass of the pharmaceutical composition. In certain embodiments, the total Ac-Di-Sol mass is about 1%, 2%, 3%, 4%, 5%, 6%, 7%, 8%, 9%, 10%, 11%, 12%, 13%, 14%, 15%, or 16% of the total mass of the pharmaceutical composition.

In certain embodiments, the solid dosage forms provided herein comprise PVPP. In certain embodiments, the total PVPP mass is at least 5%, 6%, 7%, 8%, 9%, 10%, 11%, 12%, 13%, 14%, 15%, 16%, 17%, 18%, 19%, 20%, 21%, 22%, 23%, 24%, or 25% of the total mass of the pharmaceutical composition. In certain embodiments, the total PVPP mass is no more than 5%, 6%, 7%, 8%, 9%, 10%, 11%, 12%, 13%, 14%, 15%, 16%, 17%, 18%, 19%, 20%, 21%, 22%, 23%, 24%, or 25% of the total mass of the pharmaceutical composition. In certain embodiments, the total PVPP mass is about 5%, 6%, 7%, 8%, 9%, 10%, 11%, 12%, 13%, 14%, 15%, 16%, 17%, 18%, 19%, 20%, 21%, 22%, 23%, 24%, or 25% of the total mass of the pharmaceutical composition.

In certain embodiments, the solid dosage forms provided herein comprise: (i) a pharmaceutical agent having a total pharmaceutical agent mass that is at least 5% and no more than 35% of the total mass of the pharmaceutical composition, (ii) L-HPC (e.g., L-HPC of grade LH-B1) having a total L-HPC mass that is at least 22% (e.g., at least 22%, 23%, 24%, 25%, 26%, 27%, 28%, 29%, 30%, 31%, 32%, 33%, 34%, 35%, 36%, 37%, 38%, 39%, 40%, 41%, or 42%) and no more than 42% (e.g., no more than 22%, 23%, 24%, 25%, 26%, 27%, 28%, 29%, 30%, 31%, 32%, 33%, 34%, 35%, 36%, 37%, 38%, 39%, 40%, 41%, or 42%) of the total mass of the pharmaceutical composition; (iii) Ac-Di-Sol (e.g., Ac-Di-Sol of grade SD-711) having a total Ac-Di-Sol mass that is at least 0.01% (e.g., at least 0.01%, 0.1%, 1%, 2%, 3%, 4%, 5%, 6%, 7%, 8%, 9%, 10%, 11%, 12%, 13%, 14%, 15%, or 16%) and no more than 16% (e.g., no more than 1%, 2%, 3%, 4%, 5%, 6%, 7%, 8%, 9%, 10%, 11%, 12%, 13%, 14%, 15%, or 16%) of the total mass of the pharmaceutical composition; and (iv) PVPP having a total PVPP mass that is at least 5% (e.g., at least 5%, 6%, 7%, 8%, 9%, 10%, 11%, 12%, 13%, 14%, 15%, 16%, 17%, 18%, 19%, 20%, 21%, 22%, 23%, 24%, or 25%) and no more than 25% (no more than 5%, 6%, 7%, 8%, 9%, 10%, 11%, 12%, 13%, 14%, 15%, 16%, 17%, 18%, 19%, 20%, 21%, 22%, 23%, 24%, or 25%) of the total mass of the pharmaceutical composition. In certain embodiments, the total L-HPC mass plus the total Ac-Di-Sol mass plus the total PVPP mass is at least 35%, 40%, 45%, or 50% of the total mass of the pharmaceutical composition. In some embodiments, the solid dosage form comprises: a total L-HPC mass is about 32% of the total mass of the pharmaceutical composition; a total Ac-Di-Sol mass is about 6% of the total mass of the pharmaceutical composition; and a total PVPP mass is about 15% of the total mass of the pharmaceutical composition.

In certain embodiments, the solid dosage forms provided herein further comprise mannitol. In certain embodiments, the mannitol is (or comprises) mannitol SD200. In certain embodiments, the total mannitol mass is at least 10%, 11%, 12%, 13%, 14%, 15%, 16%, 17%, 18%, 19%, 20%, 21%, 22%, 23%, 24%, 25%, 26%, 27%, 28%, 29% or 30% of the total mass of the pharmaceutical composition. In certain embodiments, the total mannitol mass is no more than 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% of the total mass of the pharmaceutical composition. In certain embodiments, the total mannitol mass is about 10%, 11%, 12%, 13%, 14%, 15%, 15.5%, 16%, 16.5%, 17%, 17.5%, 18%, 18.5%, 19%, 19.5%, 20%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5% or 40% of the total mass of the pharmaceutical composition.

In certain embodiments, the solid dosage forms provided herein comprise magnesium stearate. In certain embodiments, the total magnesium stearate mass is at least 0.1%, 1%, 2%, 3%, 4%, 5%, 6%, 7%, 8%, 9%, 10%, or 11% of the total mass of the pharmaceutical composition. In certain embodiments, the total magnesium stearate mass is no more than 0.01%, 0.1%, 1%, 2%, 3%, 4%, 5%, 6%, 7%, 8%, 9%, 10%, or 11% of the total mass of the pharmaceutical composition. In certain embodiments, the total magnesium stearate mass is about 0.01%, 0.1%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, or 11% of the total mass of the pharmaceutical composition.

In certain embodiments, the solid dosage forms provided herein comprise colloidal silica. In certain embodiments, the colloidal silica is (or comprises) Aerosil 200. In certain embodiments, the total colloidal silica mass is at least 0.01%, 0.1%, 1%, 2%, 3%, 4%, 5%, 6%, 7%, 8%, 9%, 10%, or 11% of the total mass of the pharmaceutical composition. In certain embodiments, the total colloidal silica mass is no more than 0.01%, 1%, 2%, 3%, 4%, 5%, 6%, 7%, 8%, 9%, 10%, or 11% of the total mass of the pharmaceutical composition. In certain embodiments, the total colloidal silica mass is about 0.1%, 1%, 2%, 3%, 4%, 5%, 6%, 7%, 8%, 9%, 10%, or 11% of the total mass of the pharmaceutical composition.

In certain aspects provided herein are solid dosage forms of pharmaceutical compositions. In certain embodiments, the solid dosage form comprises a pharmaceutical agent, wherein the pharmaceutical agent comprises bacteria and/or mEVs (such as smEVs and/or pmEVs) and a spirulina component and a diluent. In certain embodiments, the total pharmaceutical agent mass is at least 5%, 10%, 15%, 20%, 25%, 30%, 35%, 40%, 45%, 50%, or 55%, 60%, 65%, 70%, 75%, 80%, 85%, 90%, or 95% of the total mass of the pharmaceutical composition. In some embodiments, the total pharmaceutical agent mass is no more than 95%, 90%, 85%, 80%, 75%, 70%, 65%, 60%, 55%, 50%, 45%, 40%, 35%, 30%, 25%, 20%, 15%, 10%, or 5% of the total mass of the pharmaceutical composition.

In some embodiments, the total mass of the diluent is at least 1%, 5%, 10%, 15%, 20%, 25%, 30%, 35%, 40%, 45%, 50%, 55%, 60%, 65%, 70%, 75%, 80%, 85%, 90%, or 95% of the total mass of the pharmaceutical composition. In some embodiments, the total mass of the diluent is no more than 95%, 90%, 85%, 80%, 75%, 70%, 65%, 60%, 55%, 50%, 45%, 40%, 35%, 30%, 25%, 20%, 15%, 10%, 5%, or 1% of the total mass of the pharmaceutical composition. In some embodiments, the diluent comprises mannitol.

In certain embodiments, the solid dosage form provided herein comprises a lubricant. In certain embodiments, the total lubricant mass is at least 0.1%, 0.5%, 1%, 2%, 3%, 4%, or 5% of the total mass of the pharmaceutical composition. In certain embodiments, the total lubricant mass is no more than 0.1%, 0.5%, 1%, 2%, 3%, 4%, or 5% of the total mass of the pharmaceutical composition. In certain embodiments, the total lubricant mass is about 0.1%, 0.5%, 1%, 2%, 3%, 4%, or 5% of the total mass of the pharmaceutical composition. In certain embodiments, the total lubricant mass is about 0.5% to about 1.5% of the total mass of the pharmaceutical composition. In certain embodiments, the total lubricant mass is about 1% of the total mass of the pharmaceutical composition. In some embodiments, the lubricant comprises magnesium stearate.

In certain embodiments, the solid dosage forms provided herein comprise a glidant. In some embodiments, the glidant is colloidal silicon dioxide. In certain embodiments, the total glidant mass is at least 0.01%, 0.1%, 0.2%, 0.3%, 0.4%, 0.5%, 0.7%, 0.8%, 0.9%, 1%, 1.5%, or 2% of the total mass of the pharmaceutical composition. In certain embodiments, the total glidant mass is no more than 0.01%, 0.1%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is about 0.01%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is about 0.25% to about 0.75% of the total mass of the pharmaceutical composition. In certain embodiments, the total glidant mass is about 0.5% of the total mass of the pharmaceutical composition.

In certain embodiments, the solid dosage forms provided herein comprise: (i) a pharmaceutical agent having a total pharmaceutical agent mass that is about 20% to about 50% of the total mass of the pharmaceutical composition; (ii) a diluent (e.g., mannitol) having a total mass that is about 50% to 80% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is at least 5% and no more than 95% of the total mass of the pharmaceutical composition; (ii) a diluent (e.g., mannitol) having a total mass that is at least 1% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 8% to about 92% of the total mass of the pharmaceutical composition; (ii) a diluent (e.g., mannitol) having a total mass that is about 5% to 90% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 30% to about 50% of the total mass of the pharmaceutical composition; (ii) a diluent (e.g., mannitol) having a total mass that is about 45% to 70% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 50% of the total mass of the pharmaceutical composition; (ii) a diluent (e.g., mannitol) having a total mass that is about 48.5% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition. In certain embodiments, the solid dosage forms provided herein comprise: (i) a pharmaceutical agent having a total pharmaceutical agent mass that is about 13.51% of the total mass of the pharmaceutical composition; (ii) a diluent (e.g., mannitol) having a total mass that is about 84.99% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 90.22% of the total mass of the pharmaceutical composition; (ii) a diluent (e.g., mannitol) having a total mass that is about 8.28% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

Thus, in certain embodiments, provided herein are solid dosage forms comprising a pharmaceutical agent that contains bacteria. The bacteria can be live bacteria (e.g., powder or biomass thereof); non-live (dead) bacteria (e.g., powder or biomass thereof); non replicating bacteria (e.g., powder or biomass thereof); gamma irradiated bacteria (e.g., powder or biomass thereof); and/or lyophilized bacteria (e.g., powder or biomass thereof).

In certain embodiments, provided herein are solid dosage forms comprising a pharmaceutical agent that contains mEVs. The mEVs can be from culture media (e.g., culture supernatant). The mEVs can be from live bacteria (e.g., powder or biomass thereof); the mEVs can be from non-live (dead) bacteria (e.g., powder or biomass thereof); the mEVs can be from non-replicating bacteria (e.g., powder or biomass thereof); the mEVs can be from gamma irradiated bacteria (e.g., powder or biomass thereof); and/or the mEVs can be from lyophilized bacteria (e.g., powder or biomass thereof).

In some embodiments, the pharmaceutical agent comprises mEVs substantially or entirely free of bacteria (e.g., whole bacteria), bacteria (e.g., live bacteria, dead (e.g., killed), non-replicating bacteria, attenuated bacteria. In some embodiments, the pharmaceutical compositions comprise both mEVs and bacteria (e.g., whole bacteria) (e.g., live bacteria, killed bacteria, attenuated bacteria). In some embodiments, the pharmaceutical agents comprise bacteria and/or mEVs from one or more (e.g., 1, 2, 3, 4, 5, 6, 7, 8, 9, 10, or more) of the hemoglobin-dependent bacteria strains. In some embodiments, the pharmaceutical agents comprise bacteria and/or mEVs from one of the bacteria strains or species described herein, e.g., from bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, Turicibacter, or Veillonella.

In some embodiments, the pharmaceutical agents comprise lyophilized bacteria and/or mEVs. In some embodiments, the pharmaceutical agent comprises gamma irradiated bacteria and/or mEVs. The mEVs (such as smEVs and/or pmEVs) can be gamma irradiated after the mEVs are isolated (e.g., prepared).

In some embodiments, to quantify the numbers of mEVs (such as smEVs and/or pmEVs) and/or bacteria present in a sample, electron microscopy (e.g., EM of ultrathin frozen sections) can be used to visualize the mEVs (such as smEVs and/or pmEVs) and/or bacteria and count their relative numbers. Alternatively, nanoparticle tracking analysis (NTA), Coulter counting, or dynamic light scattering (DLS) or a combination of these techniques can be used. NTA and the Coulter counter count particles and show their sizes. DLS gives the size distribution of particles, but not the concentration. Bacteria frequently have diameters of 1-2 um (microns). The full range is 0.2-20 um. Combined results from Coulter counting and NTA can reveal the numbers of bacteria and/or mEVs (such as smEVs and/or pmEVs) in a given sample. Coulter counting reveals the numbers of particles with diameters of 0.7-10 um. For most bacterial and/or mEV (such as smEV and/or pmEV) samples, the Coulter counter alone can reveal the number of bacteria and/or mEVs (such as smEVs and/or pmEVs) in a sample. pmEVs are 20-600 nm in diameter. For NTA, a Nanosight instrument can be obtained from Malvern Pananlytical. For example, the NS300 can visualize and measure particles in suspension in the size range NTA allows for counting of the numbers of particles that are, for example, 50-1000 nm in diameter. DLS reveals the distribution of particles of different diameters within an approximate range of 1 nm-3 um.

mEVs can be characterized by analytical methods known in the art (e.g., Jeppesen, et al. Cell 177:428 (2019)).

In some embodiments, the bacteria and/or mEVs may be quantified based on particle count. For example, total protein content of a bacteria and/or mEV preparation can be measured using NTA.

In some embodiments, the bacteria and/or mEVs may be quantified based on the amount of protein, lipid, or carbohydrate. For example, total protein content of a bacteria and/or preparation can be measured using the Bradford assay or BCA.

In some embodiments, mEVs are isolated away from one or more other bacterial components of the source bacteria or bacterial culture. In some embodiments, bacteria are isolated away from one or more other bacterial components of the source bacterial culture. In some embodiments, the pharmaceutical agent further comprises other bacterial components.

In certain embodiments, the mEV preparation obtained from the source bacteria may be fractionated into subpopulations based on the physical properties (e.g., sized, density, protein content, binding affinity) of the subpopulations. One or more of the mEV subpopulations can then be incorporated into the pharmaceutical agents of the invention.

In certain aspects, provided herein are pharmaceutical compositions and/or solid dosage forms comprising pharmaceutical agents that comprise bacteria and/or mEVs (such as smEVs and/or pmEVs) useful for the treatment and/or prevention of disease (e.g., a cancer, an autoimmune disease, an inflammatory disease, a metabolic disease, or a dysbiosis), as well as methods of making and/or identifying such bacteria and/or mEVs, and methods of using pharmaceutical agents and pharmaceutical compositions and/or solid dosage forms thereof (e.g., for the treatment of a cancer, an autoimmune disease, an inflammatory disease, or a metabolic disease, either alone or in combination with other therapeutics). In some embodiments, the pharmaceutical agents comprise both mEVs (such as smEVs and/or pmEVs) and bacteria (e.g., whole bacteria) (e.g., live bacteria, dead (e.g., killed) bacteria, non-replicating bacteria, attenuated bacteria). In some embodiments, the pharmaceutical agents comprise bacteria in the absence of mEVs (such as smEVs and/or pmEVs). In some embodiments, the pharmaceutical agents comprise mEVs (such as smEVs and/or pmEVs) in the absence of bacteria. In some embodiments, the pharmaceutical agents comprise mEVs (such as smEVs and/or pmEVs) and/or bacteria from hemoglobin-dependent bacteria. In some embodiments, the pharmaceutical agents comprise bacteria and/or mEVs from one of the bacteria strains or species described herein, e.g., from bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptomphilus, Peptostreptococcus, Porphyromonas, Prevotella, Propionibacterium, Rarimicrobium, Shuttleworthia, Turicibacter, or Veillonella.

In certain aspects, provided are pharmaceutical agents, and/or pharmaceutical compositions and/or solid dosage forms thereof, for administration to a subject (e.g., human subject). In some embodiments, the pharmaceutical agents are combined with additional active and/or inactive materials in order to produce a final product, which may be in single dosage unit or in a multi-dose format. In some embodiments, the pharmaceutical agent is combined with an adjuvant such as an immuno-adjuvant (e.g., a STING agonist, a TLR agonist, or a NOD agonist).

In some embodiments, the pharmaceutical composition and/or solid dosage form comprises at least one carbohydrate.

In some embodiments, the pharmaceutical composition and/or solid dosage form comprises at least one lipid. In some embodiments, the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments, the pharmaceutical composition and/or solid dosage form comprises at least one mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the pharmaceutical composition and/or solid dosage form comprises at least one vitamin. The at least one vitamin can be fat-soluble or water-soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the pharmaceutical composition and/or solid dosage form comprises an excipient. Non-limiting examples of suitable excipients include a buffering agent, a preservative, a stabilizer, a binder, a compaction agent, a lubricant, a glidant, a diluent, a dispersion enhancer, a disintegration agent, a flavoring agent, a sweetener, and a coloring agent.

Suitable excipients that can be included in the solid dosage form can be one or more pharmaceutically acceptable excipients known in the art. For example, see Rowe, Sheskey, and Quinn, eds., Handbook of Pharmaceutical Excipients, sixth ed.; 2009; Pharmaceutical Press and American Pharmacists Association.

In some embodiments, the pharmaceutical agent can be prepared as a powder (e.g., for resuspension).

In some embodiments, the pharmaceutical composition can be prepared as a powder (e.g., for resuspension).

### Tablets and Minitablets

The solid dosage form described herein can be, e.g., a tablet or a minitablet. Further, a plurality of minitablets can be in (e.g., loaded into) a capsule.

In some embodiments, the solid dosage form comprises a tablet (>4 mm) (e.g., 5 mm-17 mm). For example, the tablet is a 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm or 18 mm tablet. The size refers to the diameter of the tablet, as is known in the art. As used herein, the size of the tablet refers to the size of the tablet prior to application of an enteric coating.

In some embodiments, the solid dosage form comprises a minitablet. The minitablet can be in the size range of 1 mm-4 mm range. E.g., the minitablet can be a 1 mm minitablet, 1.5 mm minitablet, 2 mm minitablet, 3 mm minitablet, or 4 mm minitablet. The size refers to the diameter of the minitablet, as is known in the art. As used herein, the size of the minitablet refers to the size of the minitablet prior to application of an enteric coating.

The minitablets can be in a capsule. The capsule can be a size 00, size 0, size 1, size 2, size 3, size 4, or size 5 capsule. The capsule that contains the minitablets can comprise HPMC (hydroxyl propyl methyl cellulose) or gelatin. The minitablets can be inside a capsule: the number of minitablets inside a capsule will depend on the size of the capsule and the size of the minitablets. As an example, a size 0 capsule can contain 31-35 (an average of 33) minitablets that are 3 mm minitablets.

In some embodiments, the solid dosage form (e.g., tablet or minitablet) is enterically coated (e.g., comprises an enteric coating; e.g., is coated with an enteric coating).

### Capsules

The solid dosage form described herein can be a capsule.

The solid dosage forms can comprise capsules. In some embodiments, the capsule is a size 00, size 0, size 1, size 2, size 3, size 4, or size 5 capsule. In some embodiments, the capsule comprises HPMC (hydroxyl propyl methyl cellulose) or gelatin. In some embodiments, the capsule comprises HPMC (hydroxyl propyl methyl cellulose). In some embodiments, the capsule is banded.

In some embodiments, the solid dosage form (e.g., capsule) is enterically coated (e.g., comprises an enteric coating; e.g., is coated with an enteric coating).

### Exemplary Coatings

The solid dosage form (e.g., tablet or minitablet or capsule) described herein can be enterically coated, e.g., with one enteric coating layer or with two layers of enteric coating, e.g., an inner enteric coating and an outer enteric coating. The inner enteric coating and outer enteric coating are not identical (e.g., the inner enteric coating and outer enteric coating do not contain the same components in the same amounts). The enteric coating can allow for release of the pharmaceutical agent, e.g., in the small intestine, e.g., upper small intestine, e.g., duodenum and/or jejunum.

Release of the pharmaceutical agent in the small intestine, e.g., in the upper small intestine, e.g., in the duodenum, or in the jejunum, can allow the pharmaceutical agent to target and affect cells (e.g., epithelial cells and/or immune cells) located at these specific locations, e.g., which can cause a local effect in the small intestine and/or cause a systemic effect (e.g., an effect outside of the gastrointestinal tract).

EUDRAGIT is the brand name for a diverse range of polymethacrylate-based copolymers. It includes anionic, cationic, and neutral copolymers based on methacrylic acid and methacrylic/acrylic esters or their derivatives.

Examples of other materials that can be used in the enteric coating (e.g., the one enteric coating or the inner enteric coating and/or the outer enteric coating) include cellulose acetate phthalate (CAP), cellulose acetate trimellitate (CAT), poly(vinyl acetate phthalate) (PVAP), hydroxypropyl methylcellulose phthalate (HPMCP), fatty acids, waxes, shellac (esters of aleurtic acid), plastics, plant fibers, zein, AQUA-ZEIN® (an aqueous zein formulation containing no alcohol), amylose starch, starch derivatives, dextrins, methyl acrylate-methacrylic acid copolymers, cellulose acetate succinate, hydroxypropyl methyl cellulose acetate succinate (hypromellose acetate succinate), methyl methacrylate-methacrylic acid copolymers, and/or sodium alginate.

The enteric coating (e.g., the one enteric coating or the inner enteric coating and/or the outer enteric coating) can include a methacrylic acid ethyl acrylate (MAE) copolymer (1:1).

The one enteric coating can include methacrylic acid ethyl acrylate (MAE) copolymer (1:1) (such as Kollicoat MAE 100P).

The one enteric coating can include a Eudragit coplymer, e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

Other examples of materials that can be used in the enteric coating (e.g., the one enteric coating or the inner enteric coating and/or the outer enteric coating) include those described in, e.g., U.S. Pat. Nos. 6,312,728; 6,623,759; 4,775,536; 5,047,258; 5,292,522; 6,555,124; 6,638,534; U.S. 2006/0210631; U.S. 2008/200482; U.S. 2005/0271778; U.S. 2004/0028737; WO 2005/044240.

See also, e.g., U.S. Pat. No. 9,233,074, which provides pH dependent, enteric polymers that can be used with the solid dosage forms provided herein, including methacrylic acid copolymers, polyvinylacetate phthalate, hydroxypropylmethyl cellulose acetate succinate, hydroxypropylmethyl cellulose phthalate and cellulose acetate phthalate; suitable methacrylic acid copolymers include: poly(methacrylic acid, methyl methacrylate) 1:1 sold, for example, under the Eudragit L100 trade name; poly(methacrylic acid, ethyl acrylate) 1:1 sold, for example, under the Eudragit L100-55 trade name; partially-neutralized poly(methacrylic acid, ethyl acrylate) 1:1 sold, for example, under the Kollicoat MAE-100P trade name; and poly(methacrylic acid, methyl methacrylate) 1:2 sold, for example, under the Eudragit Si 00 trade name.

In certain aspects, the solid dosage form (e.g., tablet or minitablet or capsule) described herein further comprises a sub-coating. In some embodiments, the solid dosage form comprises a sub-coating, e.g., in addition to the enteric coating, e.g., the sub-coating is beneath the enteric coating (e.g., between the solid dosage form and the enteric coating). In some embodiments, the sub-coating comprises Opadry QX, e.g., Opadry QX Blue.

### Exemplary Doses

The dose of the pharmaceutical agent (e.g., in a pharmaceutical composition and/or solid dosage form) (e.g., for human subjects) is the dose per capsule or tablet or per total number of minitablets used in a capsule.

In embodiments where dose is determined by total cell count, total cell count can be determined by Coulter counter.

In some embodiments wherein the pharmaceutical agent comprises bacteria, the dose is total cell count of about 1×10⁷ to about 1×10¹³ cells (e.g., wherein cell number is determined by total cell count, which is determined by Coulter counter) per capsule or tablet or per total number of minitablets used in a capsule.

In some embodiments, wherein the pharmaceutical agent comprises bacteria, the dose is about 3×10¹⁰ or about 1.5×10¹¹ cells (e.g., wherein cell number is determined by total cell count, which is determined by Coulter counter) per capsule or tablet or total per total number of minitablets used in a capsule. In some embodiments, wherein the pharmaceutical agent comprises bacteria, the dose is about 8×10¹⁰ or about 1.6×10¹¹ cells or about 3.2×10¹¹ cells (e.g., wherein cell number is determined by total cell count, which is determined by Coulter counter) per capsule or tablet or total per total number of minitablets used in a capsule.

In some embodiments, wherein the pharmaceutical agent comprises mEVs, the dose of mEVs is about 1×10⁵ to about 2×10¹² particles (e.g., wherein particle count is determined by NTA (nanoparticle tracking analysis)), wherein the dose is per capsule or tablet or per total number of minitablets in a capsule.

In some embodiments, wherein the pharmaceutical agent comprises mEVs, the dose of mEVs is about 2×10⁶ to about 2×10¹⁶ particles (e.g., wherein particle count is determined by NTA (nanoparticle tracking analysis)), wherein the dose is per capsule or tablet or per total number of minitablets in a capsule.

In some embodiments, the pharmaceutical agent dose can be a milligram (mg) dose determined by weight the pharmaceutical agent (e.g., a powder comprising bacteria and/or an agent of bacterial origin, such as mEVs). The dose of the pharmaceutical agent is per capsule or tablet or per total number of minitablets, e.g., in a capsule.

For example, to administer a 1× dose of the pharmaceutical agent of about 400 mg, about 200 mg of the pharmaceutical agent is present per capsule and two capsules are administered, resulting in a dose of about 400 mg. The two capsules can be administered, for example, 1× or 2× daily.

For example, for a minitablet: about 0.1 to about 3.5 mg (0.1, 0.35, 1.0, 3.5 mg) of the pharmaceutical agent can be contained per minitablet. The minitablets can be inside a capsule: the number of minitablets inside a capsule will depend on the size of the capsule and the size of the minitablets. For example, an average of 33 (range of 31-35) 3 mm minitablets fit inside a size 0 capsule. As an example, 0.1-3.5 mg of the pharmaceutical agent per minitablet, the dose range will be 3.3 mg-115.5 mg (for 33 minitablets in size 0 capsule) per capsule (3.1 mg-108.5 mg for 31 minitablets in size 0 capsule) (3.5 mg-122.5 mg for 35 minitablets in size 0 capsule). Multiple capsules and/or larger capsule(s) can be administered to increase the administered dose and/or can be administered one or more times per day to increase the administered dose.

In some embodiments, the dose can be about 3 mg to about 125 mg of the pharmaceutical agent, per capsule or tablet or per total number of minitablets, e.g., in a capsule.

In some embodiments, the dose can be about 35 mg to about 1200 mg (e.g., about 35 mg, about 125 mg, about 350 mg, or about 1200 mg) of the pharmaceutical agent.

In some embodiments, the dose of the pharmaceutical agent can be about 30 mg to about 3500 mg (about 25, about 50, about 75, about 100, about 150, about 250, about 300, about 350, about 400, about 500, about 600, about 750, about 1000, about 1250, about 1300, about 2000, about 2500, about 3000, or about 3500 mg).

A human dose can be calculated appropriately based on allometric scaling of a dose administered to a model organism (e.g., mouse).

In some embodiments, one or two tablets capsules can be administered one or two times a day.

The pharmaceutical agent contains the bacteria and/or an agent of bacterial origin, such as mEVs, or contains a powder comprising bacteria and/or an agent of bacterial origin, such as mEVs, and can also contain one or more additional components, such as a cryoprotectant, etc.

In some embodiments, the mg (by weight) dose of the pharmaceutical agent is, e.g., about 1 mg to about 500 mg per capsule, or per tablet, or per total number of minitablets, e.g., used in a capsule.

Exemplary Methods of Using Pharmaceutical Compositions and/or Solid Dosage Forms

The pharmaceutical compositions and/or solid dosage forms described herein allow, e.g., for oral administration of a pharmaceutical agent contained therein.

The solid dosage forms having the disclosed combinations and/or amounts of disintegration agents provide a decrease in disintegration times (e.g., 2-fold, 4-fold, 6-fold, 8-fold), which can further result in an increase in therapeutic efficacy and/or physiological effect as compared to the same solid dosage forms that do not have the disclosed combinations of the disintegration agents.

The pharmaceutical composition and/or solid dosage forms described herein can be used in the treatment and/or prevention of a cancer, inflammation, autoimmunity, a metabolic condition, or a dysbiosis.

The solid dosage forms described herein can be used in the treatment and/or prevention of bacterial septic shock, cytokine storm and/or viral infection (such as a coronavirus infection, an influenza infection, and/or a respiratory syncytial virus infection).

The solid dosage forms described herein can be used to decrease inflammatory cytokine expression (e.g., decreased IL-8, IL-6, IL-1β, and/or TNFα expression levels).

Methods of using a pharmaceutical composition and/or solid dosage form (e.g., for oral administration) (e.g., for pharmaceutical use) comprising a pharmaceutical agent (e.g., a therapeutically effective amount thereof), wherein the pharmaceutical agent comprises bacteria and/or microbial extracellular vesicles (mEVs) and a spirulina component, and wherein the solid dosage form further comprises the disclosed disintegration agents are described herein.

The methods and administered pharmaceutical composition and/or solid dosage forms described herein allow, e.g., for oral administration of a pharmaceutical agent contained therein. The pharmaceutical composition and/or solid dosage form can be administered to a subject is a fed or fasting state. The pharmaceutical composition and/or solid dosage form can be administered, e.g., on an empty stomach (e.g., one hour before eating or two hours after eating). The pharmaceutical composition and/or solid dosage form can be administered one hour before eating. The pharmaceutical composition and/or solid dosage form can be administered two hours after eating.

A pharmaceutical composition and/or solid dosage form for use in the treatment and/or prevention of a cancer, inflammation, autoimmunity, a metabolic condition, or a dysbiosis is provided herein.

Use of a pharmaceutical composition and/or solid dosage form for the preparation of a medicament for the treatment and/or prevention of a cancer, inflammation, autoimmunity, a metabolic condition, or a dysbiosis is provided herein.

Use of a solid dosage form for the preparation of a medicament for the treatment and/or prevention of bacterial septic shock, cytokine storm and/or viral infection (such as a coronavirus infection, an influenza infection, and/or a respiratory syncytial virus infection) is provided herein.

Use of a solid dosage form for the preparation of a medicament for decreasing inflammatory cytokine expression (e.g., decreased IL-8, IL-6, IL-1β, and/or TNFα expression levels) is provided herein.

### Method of Making Solid Dosage Forms

In certain aspects, provided herein are methods of preparing a solid dosage form of a pharmaceutical composition, the method comprising (a) combining into a pharmaceutical composition (i) a pharmaceutical agent (e.g., comprising bacteria and/or mEVs (such as smEVs and/or pmEVs) and a spirulina component disclosed herein or a powder comprising bacteria and/or mEVs (such as smEVs and/or pmEVs and comprising at least one component of spirulina), and (ii) at least one diluent, at least one lubricant, at least one glidant, and/or at least one (e.g., one, two, or three) disintegration agent. In some embodiments, the at least one diluent comprises mannitol. In some embodiments, the at least one lubricant comprises magnesium stearate. In some embodiments, the at least one glidant comprises colloidal silicon dioxide. In some embodiments, the at least one disintegration agent comprises low-substituted hydroxypropyl cellulose (L-HPC), croscarmellose sodium (Ac-Di-Sol), and/or crospovidone (PVPP).

In certain embodiments, the total pharmaceutical agent mass is at least 5%, 10%, 15%, 20% or 25% of the total mass of the pharmaceutical composition. In some embodiments the total pharmaceutical agent mass is no more than 45%, 40%, 35%, 30%, or 25% of the total mass of the pharmaceutical composition. In some embodiments, the total mass of the one or more disintegrating agents is at least 30%, at least 35%, at least 40%, at least 45%, or at least 50% of the total mass of the pharmaceutical composition. In some embodiments, the total mass of the one or more disintegrating agents is no more than 70%, 65%, 60%, or 55% of the total mass of the pharmaceutical composition.

In some embodiments, the one or more disintegration agents comprise low-substituted hydroxypropyl cellulose (L-HPC), croscarmellose sodium (Ac-Di-Sol), and/or crospovidone (PVPP). In certain embodiments, the solid dosage forms provided herein comprise L-HPC. In some embodiments, the L-HPC is of grade LH-B1. In certain embodiments, the total L-HPC mass is at least 22%, 23%, 24%, 25%, 26%, 27%, 28%, 29%, 30%, 31%, 32%, 33%, 34%, 35%, 36%, 37%, 38%, 39%, 40%, 41%, or 42% of the total mass of the pharmaceutical composition. In certain embodiments, the total L-HPC mass is no more than 22%, 23%, 24%, 25%, 26%, 27%, 28%, 29%, 30%, 31%, 32%, 33%, 34%, 35%, 36%, 37%, 38%, 39%, 40%, 41%, or 42% of the total mass of the pharmaceutical composition. In certain embodiments, the total L-HPC mass is about 22%, 23%, 24%, 25%, 26%, 27%, 28%, 29%, 30%, 31%, 32%, 33%, 34%, 35%, 36%, 37%, 38%, 39%, 40%, 41%, or 42% of the total mass of the pharmaceutical composition. In certain embodiments, the solid dosage forms provided herein comprise Ac-Do-Sol. In some embodiments, the Ac-Di-Sol is of grade SD-711. In certain embodiments, the total Ac-Di-Sol mass is at least 0.01%, 0.1%, 1%, 2%, 3%, 4%, 5%, 6%, 7%, 8%, 9%, 10%, 11%, 12%, 13%, 14%, 15%, or 16% of the total mass of the pharmaceutical composition. In certain embodiments, the total Ac-Di-Sol mass is no more than 1%, 2%, 3%, 4%, 5%, 6%, 7%, 8%, 9%, 10%, 11%, 12%, 13%, 14%, 15%, or 16% of the total mass of the pharmaceutical composition. In certain embodiments, the total Ac-Di-Sol mass is about 1%, 2%, 3%, 4%, 5%, 6%, 7%, 8%, 9%, 10%, 11%, 12%, 13%, 14%, 15%, or 16% of the total mass of the pharmaceutical composition. In certain embodiments, the solid dosage forms provided herein comprise PVPP. In certain embodiments, the total PVPP mass is at least 5%, 6%, 7%, 8%, 9%, 10%, 11%, 12%, 13%, 14%, 15%, 16%, 17%, 18%, 19%, 20%, 21%, 22%, 23%, 24%, or 25% of the total mass of the pharmaceutical composition. In certain embodiments, the total PVPP mass is no more than 5%, 6%, 7%, 8%, 9%, 10%, 11%, 12%, 13%, 14%, 15%, 16%, 17%, 18%, 19%, 20%, 21%, 22%, 23%, 24%, or 25% of the total mass of the pharmaceutical composition. In certain embodiments, the total PVPP mass is about 5%, 6%, 7%, 8%, 9%, 10%, 11%, 12%, 13%, 14%, 15%, 16%, 17%, 18%, 19%, 20%, 21%, 22%, 23%, 24%, or 25% of the total mass of the pharmaceutical composition.

In certain embodiments, the method further comprises compressing the pharmaceutical composition, thereby forming a tablet or a minitablet. In some embodiments, the method further comprises enterically coating the tablet or minitablet, thereby preparing the enterically coated tablet. In certain embodiments, the method further comprises loading the minitablets into a capsule.

The methods of preparing a solid dosage form of a pharmaceutical composition can comprise blending, encapsulation, banding, and coating of capsules.

In certain aspects, provided herein are methods of preparing a solid dosage form of a pharmaceutical composition, the method comprising combining (e.g., blending) into a pharmaceutical composition a pharmaceutical agent (e.g., comprising bacteria and/or mEVs (such as smEVs and/or pmEVs) and a spirulina component disclosed herein or a powder comprising bacteria and/or mEVs (such as smEVs and/or pmEVs) and a spirulina component disclosed herein) and one or more additional components described herein. In certain aspects, provided herein are methods of preparing a solid dosage form of a pharmaceutical composition, the method comprising combining into a pharmaceutical composition a pharmaceutical agent (e.g., comprising bacteria and/or mEVs (such as smEVs and/or pmEVs) and a spirulina component disclosed herein or a powder comprising bacteria and/or mEVs (such as smEVs and/or pmEVs) and a spirulina component disclosed herein) and a diluent. In certain embodiments, the total pharmaceutical agent mass is at least 20%, 25%, 30%, 35%, 40%, 45%, 50% or 55% of the total mass of the pharmaceutical composition. In some embodiments the total pharmaceutical agent mass is no more than 55%, 50%, 45%, 40%, 35%, 30%, 25%, or 20% of the total mass of the pharmaceutical composition.

In some embodiments, the total mass of the diluent is at least 45%, 50%, 55%, 60%, 65%, 70%, 75%, or 80% of the total mass of the pharmaceutical composition. In some embodiments, the total mass of the diluent is no more than 80%, 75%, 70%, 65%, 60%, 55%, 50%, or 45% of the total mass of the pharmaceutical composition. In some embodiments, the diluent comprises mannitol.

In certain embodiments, the method further comprises combining a lubricant. In certain embodiments, the total lubricant mass is at least 0.1%, 0.5%, 1%, 2%, 3%, 4%, or 5% of the total mass of the pharmaceutical composition. In certain embodiments, the total lubricant mass is no more than 0.1%, 0.5%, 1%, 2%, 3%, 4%, or 5% of the total mass of the pharmaceutical composition. In certain embodiments, the total lubricant mass is about 0.1%, 0.5%, 1%, 2%, 3%, 4%, or 5% of the total mass of the pharmaceutical composition. In certain embodiments, the total lubricant mass is about 0.5% to about 1.5% of the total mass of the pharmaceutical composition. In certain embodiments, the total lubricant mass is about 1% of the total mass of the pharmaceutical composition. In some embodiments, the lubricant comprises magnesium stearate.

In certain embodiments, the method further comprises combining a glidant. In some embodiments, the glidant is colloidal silicon dioxide. In certain embodiments, the total glidant mass is at least 0.01%, 0.1%, 0.2%, 0.3%, 0.4%, 0.5%, 0.6%, 0.7%, 0.8%, 1%, 1.5%, or 2% of the total mass of the pharmaceutical composition. In certain embodiments, the total glidant mass is no more than 0.01%, 0.1%, 0.2%, 0.3%, 0.4%, 0.5%, 0.7%, 0.8%, 0.9%, 1%, 1.5%, or 2% of the total mass of the pharmaceutical composition. In certain embodiments, the total glidant mass is about 0.01%, 0.1%, 0.2%, 0.4%, 0.5%, 0.6%, 0.7%, 0.8%, 0.9%, 1%, 1.5%, or 2% of the total mass of the pharmaceutical composition. In certain embodiments, the total glidant mass is about 0.25% to about 0.75% of the total mass of the pharmaceutical composition. In certain embodiments, the total glidant mass is about 0.5% of the total mass of the pharmaceutical composition.

In certain embodiments, the method comprises combining: (i) a pharmaceutical agent having a total pharmaceutical agent mass that is at least 20% and no more than 55% of the total mass of the pharmaceutical composition, (ii) a diluent (e.g., mannitol) having a total mass that is at least 45% and no more than 80% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method comprises combining: (i) a pharmaceutical agent having a total pharmaceutical agent mass that is about 20% to about 50% of the total mass of the pharmaceutical composition, (ii) a diluent (e.g., mannitol) having a total mass that is about 50% to 80% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In some embodiments, the method further comprises loading the pharmaceutical composition into a capsule (e.g., encapsulation).

In some embodiments, the method further comprises banding the capsule after loading.

In some embodiments, the method further comprises enterically coating the capsule.

### Additional Aspects of the Solid Dosage Forms

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs) and comprises at least one component of spirulina (e.g., a spirulina component), and wherein the solid dosage form further comprises the described disintegration agents, can provide a therapeutically effective amount of the pharmaceutical agent to a subject, e.g., a human.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs) and comprises at least one component of spirulina, and wherein the solid dosage form further comprises the described disintegration agents, can provide a non-natural amount of the therapeutically effective components (e.g., present in the pharmaceutical agent) to a subject, e.g., a human.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs) and comprises at least one component of spirulina, and wherein the solid dosage form further comprises the described disintegration agents, can provide an unnatural quantity of the therapeutically effective components (e.g., present in the pharmaceutical agent) to a subject, e.g., a human.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs) and comprises at least one component of spirulina, and wherein the solid dosage form further comprises the described disintegration agents, can bring about one or more changes to a subject, e.g., human, e.g., to treat or prevent a disease or a health disorder.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs) and comprises at least one component of spirulina, and wherein the solid dosage form further comprises the described disintegration agents, has potential for significant utility, e.g., to affect a subject, e.g., a human, e.g., to treat or prevent a disease or a health disorder.

### Administration

In certain aspects, provided herein is a method of delivering a pharmaceutical composition and/or a solid dosage form described herein to a subject.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, for example, tumor size, and other compounds such as drugs being administered concurrently or near-concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of a pharmaceutical agent (e.g., in a solid dosage form and/or a pharmaceutical composition) described herein may be appropriately set or adjusted in accordance with the dosage form, the route of administration, the degree or stage of a target disease, and the like.

In some embodiments, the dose administered to a subject is sufficient to prevent disease (e.g., autoimmune disease, inflammatory disease, metabolic disease, or cancer), delay its onset, or slow or stop its progression, or relieve one or more symptoms of the disease. One skilled in the art will recognize that dosage will depend upon a variety of factors including the strength of the particular agent (e.g., pharmaceutical agent) employed, as well as the age, species, condition, and body weight of the subject. The size of the dose will also be determined by the route, timing, and frequency of administration as well as the existence, nature, and extent of any adverse side-effects that might accompany the administration of a particular pharmaceutical agent and the desired physiological effect.

In accordance with the above, in therapeutic applications, the dosages of the pharmaceutical agents used in accordance with the invention vary depending on the active agent, the age, weight, and clinical condition of the recipient patient, and the experience and judgment of the clinician or practitioner administering the therapy, among other factors affecting the selected dosage. For example, for cancer treatment, the dose should be sufficient to result in slowing, and preferably regressing, the growth of a tumor and most preferably causing complete regression of the cancer, or reduction in the size or number of metastases As another example, the dose should be sufficient to result in slowing of progression of the disease for which the subject is being treated, and preferably amelioration of one or more symptoms of the disease for which the subject is being treated.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a solid dosage form, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be no more than the time period for a subject to mount an immune response, such as no more than about one week, no more than about ten days, no more than about two weeks, or no more than about a month.

### Immune Disorders

In some embodiments, the methods and pharmaceutical compositions and/or solid dosage forms described herein relate to the treatment or prevention of a disease or disorder associated a pathological immune response, such as an autoimmune disease, an allergic reaction and/or an inflammatory disease. In some embodiments, the disease or disorder is an inflammatory bowel disease (e.g., Crohn's disease or ulcerative colitis). In some embodiments, the disease or disorder is psoriasis. In some embodiments, the disease or disorder is psoriatic arthritis. In some embodiments, the disease or disorder is atopic dermatitis. In some embodiments, the disease or disorder is asthma.

The methods and pharmaceutical compositions and/or solid dosage forms described herein can be used to treat any subject in need thereof. As used herein, a “subject in need thereof” includes any subject that has a disease or disorder associated with a pathological immune response (e.g., an inflammatory bowel disease), as well as any subject with an increased likelihood of acquiring a such a disease or disorder.

The pharmaceutical compositions and/or solid dosage forms described herein can be used, for example, as a pharmaceutical composition for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma; an infectious disease, such as an infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a supplement, food, or beverage for improving immune functions; or a reagent for suppressing the proliferation or function of immune cells.

In some embodiments, the methods and pharmaceutical compositions and/or solid dosage forms provided herein are useful for the treatment of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to a immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the methods and solid dosage forms described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and solid dosage forms described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and solid dosage forms described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and pharmaceutical compositions and/or solid dosage forms described herein may be used to treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, goodpasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and pharmaceutical compositions and/or solid dosage forms described herein may be used to treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dustmite allergy) and gluten-sensitive enteropathy (Celiac disease).

Other immune disorders which may be treated with the methods and solid dosage forms include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Metabolic Disorders

In some embodiments, the methods and pharmaceutical compositions and/or solid dosage forms described herein relate to the treatment or prevention of a metabolic disease or disorder a, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH) or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema. In some embodiments, the methods and pharmaceutical compositions described herein relate to the treatment of Nonalcoholic Fatty Liver Disease (NAFLD) and Nonalcoholic Steatohepatitis (NASH).

The methods and pharmaceutical compositions and/or solid dosage forms described herein can be used to treat any subject in need thereof. As used herein, a “subject in need thereof” includes any subject that has a metabolic disease or disorder, as well as any subject with an increased likelihood of acquiring a such a disease or disorder.

The pharmaceutical compositions and/or solid dosage forms described herein can be used, for example, for preventing or treating (reducing, partially or completely, the adverse effects of) a metabolic disease, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH), or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema.

### Cancer

In some embodiments, the methods and pharmaceutical compositions and/or solid dosage forms described herein relate to the treatment of cancer. In some embodiments, any cancer can be treated using the methods described herein. Examples of cancers that may treated by methods and solid dosage forms described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer comprises a solid tumor.

In some embodiments, the cancer comprises breast cancer (e.g., triple negative breast cancer).

In some embodiments, the cancer comprises colorectal cancer (e.g., microsatellite stable (MSS) colorectal cancer).

In some embodiments, the cancer comprises renal cell carcinoma.

In some embodiments, the cancer comprises lung cancer (e.g., non small cell lung cancer).

In some embodiments, the cancer comprises bladder cancer.

In some embodiments, the cancer comprises gastroesophageal cancer.

In some embodiments, the methods and pharmaceutical compositions and/or solid dosage forms provided herein relate to the treatment of a leukemia. The term “leukemia” includes broadly progressive, malignant diseases of the hematopoietic organs/systems and is generally characterized by a distorted proliferation and development of leukocytes and their precursors in the blood and bone marrow. Non-limiting examples of leukemia diseases include, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

In some embodiments, the methods and pharmaceutical compositions and/or solid dosage forms provided herein relate to the treatment of a carcinoma. The term “carcinoma” refers to a malignant growth made up of epithelial cells tending to infiltrate the surrounding tissues, and/or resist physiological and non-physiological cell death signals and gives rise to metastases. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

In some embodiments, the methods and pharmaceutical compositions and/or solid dosage forms provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and pharmaceutical compositions and/or solid dosage forms described herein include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, plasmacytoma, colorectal cancer, rectal cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

Particular categories of tumors that can be treated using methods and pharmaceutical compositions and/or solid dosage forms described herein include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, plasmacytoma, colorectal cancer, and rectal cancer.

Cancers treated in certain embodiments also include precancerous lesions, e.g., actinic keratosis (solar keratosis), moles (dysplastic nevi), acitinic chelitis (farmer's lip), cutaneous horns, Barrett's esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

### Other Diseases and Disorders

In some embodiments, the methods and pharmaceutical compositions and/or solid dosage forms described herein relate to the treatment of liver diseases. Such diseases include, but are not limited to, Alagille Syndrome, Alcohol-Related Liver Disease, Alpha-1 Antitrypsin Deficiency, Autoimmune Hepatitis, Benign Liver Tumors, Biliary Atresia, Cirrhosis, Galactosemia, Gilbert Syndrome, Hemochromatosis, Hepatitis A, Hepatitis B, Hepatitis C, Hepatic Encephalopathy, Intrahepatic Cholestasis of Pregnancy (ICP), Lysosomal Acid Lipase Deficiency (LAL-D), Liver Cysts, Liver Cancer, Newborn Jaundice, Primary Biliary Cholangitis (PBC), Primary Sclerosing Cholangitis (PSC), Reye Syndrome, Type I Glycogen Storage Disease, and Wilson Disease.

The methods and pharmaceutical compositions and/or solid dosage forms described herein may be used to treat neurodegenerative and neurological diseases. In certain embodiments, the neurodegenerative and/or neurological disease is Parkinson's disease, Alzheimer's disease, prion disease, Huntington's disease, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy or post-operative cognitive dysfunction.

### Dysbiosis

In recent years, it has become increasingly clear that the gut microbiome (also called the “gut microbiota”) can have a significant impact on an individual's health through microbial activity and influence (local and/or distal) on immune and other cells of the host (Walker, W. A., Dysbiosis. The Microbiota in Gastrointestinal Pathophysiology. Chapter 25. 2017; Weiss and Thierry, Mechanisms and consequences of intestinal dysbiosis. Cellular and Molecular Life Sciences. (2017) 74(16):2959-2977. Zurich Open Repository and Archive, doi: https://doi.org/10.1007/s00018-017-2509-x)).

A healthy host-gut microbiome homeostasis is sometimes referred to as a “eubiosis” or “normobiosis,” whereas a detrimental change in the host microbiome composition and/or its diversity can lead to an unhealthy imbalance in the microbiome, or a “dysbiosis” (Hooks and O'Malley. Dysbiosis and its discontents. American Society for Microbiology. Oct 2017. Vol. 8. Issue 5. mBio 8:e01492-17. https://doi.org/10.1128/mBio.01492-17). Dysbiosis, and associated local or distal host inflammatory or immune effects, may occur where microbiome homeostasis is lost or diminished, resulting in: increased susceptibility to pathogens; altered host bacterial metabolic activity; induction of host proinflammatory activity and/or reduction of host anti-inflammatory activity. Such effects are mediated in part by interactions between host immune cells (e.g., T cells, dendritic cells, mast cells, NK cells, intestinal epithelial lymphocytes (IEC), macrophages and phagocytes) and cytokines, and other substances released by such cells and other host cells.

A dysbiosis may occur within the gastrointestinal tract (a “gastrointestinal dysbiosis” or “gut dysbiosis”) or may occur outside the lumen of the gastrointestinal tract (a “distal dysbiosis”). Gastrointestinal dysbiosis is often associated with a reduction in integrity of the intestinal epithelial barrier, reduced tight junction integrity and increased intestinal permeability. Citi, S. Intestinal Barriers protect against disease, Science 359:1098-99 (2018); Srinivasan et al., TEER measurement techniques for in vitro barrier model systems. J. Lab. Autom. 20:107-126 (2015). A gastrointestinal dysbiosis can have physiological and immune effects within and outside the gastrointestinal tract.

The presence of a dysbiosis has been associated with a wide variety of diseases and conditions including: infection, cancer, autoimmune disorders (e.g., systemic lupus erythematosus (SLE)) or inflammatory disorders (e.g., functional gastrointestinal disorders such as inflammatory bowel disease (IBD), ulcerative colitis, and Crohn's disease), neuroinflammatory diseases (e.g., multiple sclerosis), transplant disorders (e.g., graft-versus-host disease), fatty liver disease, type I diabetes, rheumatoid arthritis, Sjogren's syndrome, celiac disease, cystic fibrosis, chronic obstructive pulmonary disorder (COPD), and other diseases and conditions associated with immune dysfunction. Lynch et al., The Human Microbiome in Health and Disease, N. Engl. J. Med 375:2369-79 (2016), Carding et al., Dysbiosis of the gut microbiota in disease. Microb. Ecol. Health Dis. (2015); 26: 10: 3402/mehd.v26.2619; Levy et al, Dysbiosis and the Immune System, Nature Reviews Immunology 17:219 (April 2017)

Exemplary pharmaceutical compositions and/or solid dosage forms disclosed herein can treat a dysbiosis and its effects by modifying the immune activity present at the site of dysbiosis. As described herein, such compositions can modify a dysbiosis via effects on host immune cells, resulting in, e.g., an increase in secretion of anti-inflammatory cytokines and/or a decrease in secretion of pro-inflammatory cytokines, reducing inflammation in the subject recipient or via changes in metabolite production.

Exemplary pharmaceutical compositions and/or solid dosage forms disclosed herein that are useful for treatment of disorders associated with a dysbiosis contain one or more types of immunomodulatory bacteria (e.g., anti-inflammatory bacteria) and/or mEVs (microbial extracellular vesicles) derived from such bacteria. Such compositions are capable of affecting the recipient host's immune function, in the gastrointestinal tract, and/or a systemic effect at distal sites outside the subject's gastrointestinal tract.

Exemplary pharmaceutical compositions and/or solid dosage forms disclosed herein that are useful for treatment of disorders associated with a dysbiosis contain a population of immunomodulatory bacteria of a single bacterial species (e.g., a single strain) (e.g., anti-inflammatory bacteria) and/or mEVs derived from such bacteria. Such compositions are capable of affecting the recipient host's immune function, in the gastrointestinal tract, and/or a systemic effect at distal sites outside the subject's gastrointestinal tract.

In one embodiment, pharmaceutical compositions and/or solid dosage forms containing an isolated population of immunomodulatory bacteria (e.g., anti-inflammatory bacterial cells) or mEVs derived from such bacteria are administered (e.g., orally) to a mammalian recipient in an amount effective to treat a dysbiosis and one or more of its effects in the recipient. The dysbiosis may be a gastrointestinal tract dysbiosis or a distal dysbiosis.

In another embodiment, pharmaceutical compositions and/or solid dosage forms of the instant invention can treat a gastrointestinal dysbiosis and one or more of its effects on host immune cells, resulting in an increase in secretion of anti-inflammatory cytokines and/or a decrease in secretion of pro-inflammatory cytokines, reducing inflammation in the subject recipient.

In another embodiment, the pharmaceutical compositions and/or solid dosage forms can treat a gastrointestinal dysbiosis and one or more of its effects by modulating the recipient immune response via cellular and cytokine modulation to reduce gut permeability by increasing the integrity of the intestinal epithelial barrier.

In another embodiment, the pharmaceutical compositions and/or solid dosage forms can treat a distal dysbiosis and one or more of its effects by modulating the recipient immune response at the site of dysbiosis via modulation of host immune cells.

Other exemplary pharmaceutical compositions and/or solid dosage forms are useful for treatment of disorders associated with a dysbiosis, which compositions contain one or more types of bacteria or mEVs capable of altering the relative proportions of host immune cell subpopulations, e.g., subpopulations of T cells, immune lymphoid cells, dendritic cells, NK cells and other immune cells, or the function thereof, in the recipient.

Other exemplary pharmaceutical compositions and/or solid dosage forms are useful for treatment of disorders associated with a dysbiosis, which compositions contain a population of immunomodulatory bacteria or mEVs of a single bacterial species e.g., a single strain) capable of altering the relative proportions of immune cell subpopulations, e.g., T cell subpopulations, immune lymphoid cells, NK cells and other immune cells, or the function thereof, in the recipient subject.

In one embodiment, the invention provides methods of treating a gastrointestinal dysbiosis and one or more of its effects by orally administering to a subject in need thereof a pharmaceutical composition and/or solid dosage forms which alters the microbiome population existing at the site of the dysbiosis. The pharmaceutical composition and/or solid dosage forms can contain one or more types of immunomodulatory bacteria or mEVs or a population of immunomodulatory bacteria or mEVs of a single bacterial species (e.g., a single strain).

In one embodiment, the invention provides methods of treating a distal dysbiosis and one or more of its effects by orally administering to a subject in need thereof a pharmaceutical composition and/or solid dosage forms which alters the subject's immune response outside the gastrointestinal tract. The pharmaceutical composition and/or solid dosage forms can contain one or more types of immunomodulatory bacteria or mEVs or a population of immunomodulatory bacteria or mEVs of a single bacterial species (e.g., a single strain).

In exemplary embodiments, pharmaceutical compositions and/or solid dosage forms useful for treatment of disorders associated with a dysbiosis stimulate secretion of one or more anti-inflammatory cytokines by host immune cells. Anti-inflammatory cytokines include, but are not limited to, IL-10, IL-13, IL-9, IL-4, IL-5, TGFβ, and combinations thereof. In other exemplary embodiments, pharmaceutical compositions and/or solid dosage forms useful for treatment of disorders associated with a dysbiosis that decrease (e.g., inhibit) secretion of one or more pro-inflammatory cytokines by host immune cells. Pro-inflammatory cytokines include, but are not limited to, IFNγ, IL-12p70, IL-1α, IL-6, IL-8, MCP1, MIP1α, MIP1β, TNFα, and combinations thereof. Other exemplary cytokines are known in the art and are described herein.

In another aspect, the invention provides a method of treating or preventing a disorder associated with a dysbiosis in a subject in need thereof, comprising administering (e.g., orally administering) to the subject a therapeutic composition in the form of a probiotic or medical food comprising bacteria or mEVs in an amount sufficient to alter the microbiome at a site of the dysbiosis, such that the disorder associated with the dysbiosis is treated.

In another embodiment, a pharmaceutical composition and/or solid dosage form of the instant invention in the form of a probiotic or medical food may be used to prevent or delay the onset of a dysbiosis in a subject at risk for developing a dysbiosis.

### EXAMPLES

### Example 1: Exemplary Manufacturing Process of Hemoglobin-Dependent Bacteria

An exemplary manufacturing process for preparing hemoglobin-dependent bacteria, e.g., Prevotella histicola, is presented herein. In this exemplary method, the hemoglobin-dependent bacteria are grown in growth media comprising spirulina, for example, comprising the components listed in Table 4 or 5. The media is filter sterilized prior to use.

Spirulina was prepared by powdering spirulina tablets and dissolving the powder in water or 0.01 M NaOH. The solution was sterilized by autoclaving, and was added to the growth media at various working concentrations (e.g., 0.02 g/L, 0.2 g/L, or 2 g/L).

TABLE 4
Exemplary Growth Media

| Component | g/L |
|---|---|
| Yeast Extract 19512 | 10 |
| Soy Peptone A2SC 19649 | 12.5 |
| Soy Peptone E110 19885 | 12.5 |
| Dipotassium Phosphate K2HPO4 | 1.59 |
| Monopotassium phosphate | 0.91 |
| L-Cysteine-HCl | 0.5 |
| Ammonium chloride | 0.5 |
| Glucidex 21 D (Maltodextrin) | 25 |
| Glucose | 10 |
| Spirulina | 1 |

TABLE 5
Another Exemplary Growth Media (SPYG1 media)

| Component | g/L |
|---|---|
| Yeast Extract 19512 Organotechnie S.A.S. | 10 |
| Soy Peptone A2SC 19649 Organotechnie S.A.S. | 10 |
| Soy Peptone E110 19885 Organotechnie S.A.S. | 10 |
| Dipotassium Phosphate K2HPO4 | 2.5 |
| L-Cysteine-HCl | 0.5 |
| Glucose | 5 |
| Spirulina | 1 |

Briefly, a 1 L bottle is inoculated with a 1 mL of a cell bank sample that had been stored at −80° C. This inoculated culture is incubated in an anaerobic chamber at 37° C., pH=6.5 due to sensitivity of this strain to aerobic conditions. When the bottle reaches log growth phase (after approximately 14 to 16 hours of growth), the culture is used to inoculate a 20 L bioreactor at 5% v/v. During log growth phase (after approximately 10 to 12 hours of growth), the culture is used to inoculate a 3500 L bioreactor at 0.5% v/v.

Fermentation culture is continuously mixed with addition of a mixed gas at 0.02 VVM with a composition of 25% CO₂ and 75% N₂. pH is maintained at 6.5 with ammonium hydroxide and temperature controlled at 37° C. Harvest time is based on when stationary phase is reached (after approximately 12 to 14 hours of growth).

Once fermentation complete, the culture is cooled to 10° C., centrifuged and the resulting cell paste is collected. 10% Stabilizer is added to the cell paste and mixed thoroughly (Stabilizer Concentration (in slurry): 1.5% Sucrose, 1.5% Dextran, 0.03% Cysteine). The cell slurry is lyophilized (e.g., to prepare a powder, e.g., a pharmaceutical agent). See Table 6.

For other growth conditions that can be used, see, e.g., WO 2019/051381, the disclosures of which is hereby incorporated by reference.

Hemoglobin-dependent bacteria that have been grown in media containing spirulina as a substitute for an animal hemoglobin include: Prevotella histicola, such as Prevotella Strain B 50329 (NRRL accession number B 50329) and Prevotella Strain C (PTA-126140); two strains of Fournierella, including Fournierella Strain B (PTA-126696); two strains of Parabacteroides; a strain of Faecalibacterium; a strain of Bacteroides; and a strain of Alistipes. See, e.g., WO 20211025968, the disclosure of which is hereby incorporated by reference.

TABLE 6
Stabilizer Formulation

| Component | g/kg |
|---|---|
| Sucrose | 200 |
| Dextran 40k | 200 |
| Cysteine HCl | 4 |
| Water | 596 |

### Example 2: Preparation of a Solid Dosage Form Comprising Prevotella histicola

Tableting is performed and manufactured batches are first sub-coated with Opadry QX blue before top-coating for enteric release with Kollicoat MAE100P. See Tables 7-9.

TABLE 7
Prevotella histicola Tablet Composition

| Material | Active Dose (% w/w) |
|---|---|
| Prevotella histicola Strain B (NRRL accession | 25.0 |
| number B 50329) powder | |
| Mannitol 200 SD | 19.5 |
| L-HPC (LH-B1) | 32.0 |
| Crospovidone (Kollidon CL-F) | 15.0 |
| Croscarmellose Sodium (Ac-Di-Sol SD-711) | 6.0 |
| Colloidal Silica (Aerosil 200) | 1.0 |
| MG Stearate | 1.5 |
| Total | 100.0 |

The Prevotella histicola strain referred to above has been deposited as Prevotella histicola Strain B (NRRL accession number B 50329).

The dose composition of Table 7 is provided in a 17.4 mm×7.1 mm tablet.

The Prevotella histicola Strain B (NRRL accession number B 50329) powder in Table 7 further includes at least one spirulina component.

TABLE 8
Sub-coating Composition

| Materials | (% w/w) |
|---|---|
| Opadry QX Blue | 15.00 |
| WFI | 85.00 |
| Total | 100.00 |

TABLE 9
Top-coating Composition

| Materials | (% w/w) |
|---|---|
| Kollicoat MAE 100P | 15.00 |
| TEC | 2.25 |
| Talc | 3.00 |
| Water | 79.75 |
| Total | 100 |

The target weight per tablet is 650 mg (dose strength 162.5 mg).

### Example 3: Preparation of a Capsule Comprising Prevotella histicola

The following recipe in Table 10 is prepared.

TABLE 10
Prevotella histicola Capsule Composition

| | | Reference to | |
|---|---|---|---|
| Name of ingredient(s) | Function | standards | % w/w |
| Prevotella histicola | Active | NA | 30-50%^{#} |
| (lyophilized) powder | ingredient | | |
| Mannitol | Diluent | USP/Ph. | 50-70%^{#} |
| | | Eur. | |
| Magnesium stearate | Lubricant | USP/Ph. | 1.0 |
| | | Eur. | |
| Colloidal silicon | Glidant | USP/Ph. | 0.5 |
| dioxide | | Eur. | |
| Total Fill Weight | | | 100 |
| Capsules, Size 0 | Capsule Shell | 1 unit | 1 unit |

^{#}Adjusted based on the potency of drug substance to ensure targeted strength.

The capsule is enteric coated for release at pH 5.5.

The Prevotella histicola (lyophilized) powder in Table 10 further includes at least one spirulina component.

The Prevotella histicola strain referred to above has been deposited as Prevotella histicola Strain B (NRRL accession number B 50329).

### Example 4: Preparation of a Capsule Comprising Prevotella histicola

The following recipe in Table 11 is prepared.

TABLE 11
Prevotella histicola Capsule Composition

| | | Reference to | |
|---|---|---|---|
| Name of ingredient(s) | Function | standards | % w/w |
| Prevotella histicola | Active | NA | 30-50%^{#} |
| (lyophilized) powder | ingredient | | |
| Mannitol | Diluent | USP/Ph. | 45-70%^{#} |
| | | Eur. | |
| Magnesium stearate | Lubricant | USP/Ph. | 1.0 |
| | | Eur. | |
| Colloidal silicon | Glidant | USP/Ph. | 0.5 |
| dioxide | | Eur. | |
| Total Fill Weight | | | 100 |
| Capsules, Size 0 | Capsule Shell | 1 unit | 1 unit |

^{#}Adjusted based on the potency of drug substance to ensure targeted strength.

The capsule is enteric coated for release at pH 5.5.

The Prevotella histicola (lyophilized) powder in Table 11 further includes at least one spirulina component.

The Prevotella histicola strain referred to above has been deposited as Prevotella histicola Strain B (NRRL accession number B 50329).

Batches of enteric coated capsules according to this recipe have been prepared.

### Example 5: Preparation of a Capsule Comprising Prevotella histicola

Capsules according to the following recipe in Table 12 were prepared:

TABLE 12
Prevotella histicola Capsule Composition

| Name of ingredient(s) | Function | % w/W |
|---|---|---|
| Prevotella histicola | Active | 50 |
| (lyophilized) powder | ingredient | |
| Mannitol | Diluent | 48.5 |
| Magnesium Stearate | Lubricant | 1.0 |
| Colloidal Silicon | Glidant | 0.5 |
| Dioxide | | |
| Total Fill Weight | | 100 |
| Capsules^{a}, Size 0 | Capsule | 1 unit |
| | Shell | |

^{a}Composed of hydroxypropyl methylcellulose and titanium dioxide.

This capsule contained 1.6×10¹¹ cells.

The Prevotella histicola (lyophilized) powder in Table 12 further included at least one spirulina component.

The Prevotella histicola strain referred to above has been deposited as Prevotella histicola Strain B (NRRL accession number B 50329).

The capsule was banded with an HPMC-based banding solution.

The banded capsule was enteric coated with a poly(methacrylic acid-co-ethyl acrylate copolymer.

### Example 6: Preparation of a Capsule Comprising Prevotella histicola

Capsules according to the recipe in Table 13 are prepared.

TABLE 13
Prevotella histicola Capsule Composition

| | | Reference to | |
|---|---|---|---|
| Name of ingredient(s) | Function | standards | % w/w |
| Prevotella histicola | Active | NA | 10-90%^{#} |
| (lyophilized) powder | ingredient | | |
| Mannitol | Diluent | USP/Ph. | 8.5-88.5%^{#} |
| | | Eur. | |
| Magnesium stearate | Lubricant | USP/Ph. | 1.0 |
| | | Eur. | |
| Colloidal silicon | Glidant | USP/Ph. | 0.5 |
| dioxide | | Eur. | |
| Total Fill Weight | | | 100 |
| Capsules, Size 0 | Capsule Shell | 1 unit | 1 unit |

^{#}Adjusted based on the potency of drug substance to ensure targeted strength.

The capsule is enteric coated for release at pH 5.5. The Prevotella histicola (lyophilized) powder in Table 13 further includes at least one spirulina component.

The Prevotella histicola strain referred to above has been deposited as Prevotella histicola Strain B (NRRL accession number B 50329).

Batches of enteric coated capsules according to this recipe have been prepared.

### Example 7: Preparation of a Capsule Comprising Prevotella histicola

Capsules according to the following recipe in Table 14 are prepared:

TABLE 14
Prevotella histicola Capsule Composition

| | | 1.6 × 10¹⁰ | 8.0 × 10¹⁰ | 1.6 × 10¹¹ |
|---|---|---|---|---|
| Name of | | Cells | Cells | Cells |
| ingredient(s) | Function | % w/W | % w/W | % w/w |
| Prevotella histicola | Active | 13.51 ^{b} | 90.22 ^{b} | 50 |
| (lyophilized) powder | ingredient | | | |
| Mannitol | Diluent | 84.99 ^{b} | 8.28 ^{b} | 48.5 |
| Magnesium Stearate | Lubricant | 1.0 | 1.0 | 1.0 |
| Colloidal Silicon | Glidant | 0.5 | 0.5 | 0.5 |
| Dioxide | | | | |
| Total Fill Weight | | 100 | 100 | 100 |
| Capsules^{a}, Size 0 | Capsule | 1 unit | 1 unit | 1 unit |
| | Shell | | | |

^{a}Composed of hydroxypropyl methylcellulose and titanium dioxide.
^{b} Adjusted based on the potency of drug substance to ensure targeted strength.

The Prevotella histicola (lyophilized) powder in Table 14 further includes at least one spirulina component.

The capsule is banded with an HPMC-based banding solution.

The banded capsule is enteric coated with a poly(methacrylic acid-co-ethyl acrylate copolymer.

### Example 8: Detecting Components of Spirulina

Two exemplary components of spirulina were detected in spirulina preparations. The following two components of spirulina were detected by qPCR in the preparations:
Chlorophyll a synthase (ChlG)
C-phycocyanin alpha subunit (cpcA)

The genomic sequence of spirulina was derived from NCBI Reference Sequence: NZ_AFXD00000000.1 Arthrospira platensis C1, whole genome shotgun sequencing project (world wide web at ncbi.nlm.nih.gov/nuccoreNZ AFXD00000000.1). The genome structure of A. platensis is estimated to be a single, circular chromosome of 6.8 Mb, based on optical mapping. Annotation of this 6.7 Mb sequence yielded 6630 protein-coding genes (see Fujisawa et al. (2010) DNA Res. 17:85-103).

Spirulina-specific qPCR assays were designed to target highly conserved genomic sequences (e.g., ChlG=final enzyme in the chlorophyllin biosynthesis; cpcA=c-phycocyanin subunit A) that should be minimally divergent between spirulina strains.

The following TaqMan primer/probe sets were used to detect the presence of spirulina-specific ChlG and cpcA genes:

Ch1G Set
FWD:
GCCCTATTCGGAGAACTCAAC
REV:
GATCGCCTTCGACACTCTTAAA
Probe:
CACCCTATTCTACAGCTTGGCGGG
cpcA Set
FWD:
GCTCGAAAGTCCGGTTGATT
REV:
ACCTGCGGATGGTAACTTATTG
Probe:
ACCGGCAATCAGGTACTCATCCAT

TABLE 15
qPCR Cycling Conditions

| Stage | Repetitions | Temperature | Time |
|---|---|---|---|
| 1 | 1 | 95.0° C. | 10:00 |
| 2 | 40 | 95.0° C. | 0:10 |
| | | 60.0° C. | 0:30 |

The qPCR was performed with 1.0E+06, 1.0E+04, and 1.0E+02 copies of spirulina DNA (spirulina was commercially sourced). The results indicate nearly identical amplification efficiency and sensitivity of both qPCR assays (Ch1G and cpcA). Data not shown.

### Example 9: Detecting and Quantifying the Components of Spirulina in Pharmaceutical Agents Manufactured in Different Processes

Components of spirulina were detected and quantified using qPCR in (i) Prevotella Strain B pharmaceutical agent batches manufactured using one of four processes, (ii) a Prevotella Strain B reference batch of pharmaceutical agent, and (iii) a non-hemoglobin dependent strain (a non-Prevotella strain that is not grown in growth media containing spirulina) as a negative control. Growth media for processes 1 and 2 did not contain spirulina; growth media for processes 3 and 4 did contain spirulina; growth media for the process for making Prevotella Strain B reference batch did contain spirulina.

The qPCR results are shown in Table 15. ΔCt values were calculated by subtracting ChlG and cpcA Ct values from Prevotella Strain B reference batch Ct values. Percentage values were calculated using the equation 2\hat{ }-ΔCt. As shown in Table 15, Prevotella Strain B samples manufactured using process 1 and process 2 showed undetectable levels of spirulina DNA, similar to the non-hemoglobin dependent strain sample. However, Prevotella Strain B samples manufactured using process 3 and process 4, as well as Prevotella Strain B reference batch showed varying levels of residual spirulina DNA relative to Prevotella Strain B genomic DNA.

### Example 10: Powder Preparation Sample Protocol

After desired level of bacterial culture growth is achieved, centrifuge cultures, discard the supernatant, leaving the pellet as dry as possible. Resuspend pellet in desired cryoprotectant solution to create a formulated cell paste. The cryoprotectant may contain, e.g., maltodextrin, sodium ascorbate, sodium glutamate, and/or calcium chloride. Load the formulated cell paste onto stainless steel trays and load into a freeze drier, e.g., operating in automated mode with defined cycle parameters. The freeze dried product is fed into a milling machine and the resulting powder (e.g., pharmaceutical agent) is collected.

Powders are stored (e.g., in vacuum sealed bags) at 2-8 degrees C. (e.g., at 4 degrees C.), e.g., in a desiccator.

### Example 11: Gamma-Irradiation: Sample Protocol

Powders are gamma-irradiated at 17.5 kGy radiation unit at ambient temperature. Frozen biomasses are gamma-irradiated at 25 kGy radiation unit in the presence of dry ice.

### Example 12: Tablet Comprising Prevotella histicola

A tablet with the following recipe in Table 16 was prepared.

TABLE 16
Prevotella histicola Tablet Composition

| Material | Active Dose (% w/w) |
|---|---|
| Prevotella histicola Strain B (NRRL accession | 23.0 |
| number B 50329) powder | |
| Mannitol 200 SD | 21.5 |
| L-HPC (LH-B1) | 32.0 |
| Crospovidone (Kollidon CL-F) | 15.0 |
| Croscarmellose Sodium (Ac-Di-Sol SD-711) | 6.0 |
| Colloidal Silica (Aerosil 200) | 1.0 |
| MG Stearate | 1.5 |
| Total | 100.0 |

The tablet was prepared as a 17.4 mm×7.1 mm tablet.

The tablet was enteric coated.

The tablet contained 3.2×10¹¹ TCC of Prevotella histicola Strain B (NRRL accession number B 50329).

The Prevotella histicola Strain B (NRRL accession number B 50329) powder in Table 16 further included at least one spirulina component.

The Prevotella histicola strain referred to above has been deposited as Prevotella histicola Strain B (NRRL accession number B 50329).

### Example 13: Capsule Comprising Prevotella histicola

Capsules according to the following recipe in Table 17 were prepared:

TABLE 17
Prevotella histicola Capsule Composition

| Name of | | 3.35 × 10¹¹ Cells |
|---|---|---|
| ingredient(s) | Function | % w/w |
| Prevotella histicola | Active | 50 |
| (lyophilized) powder | ingredient | |
| Mannitol (Pearlitol | Diluent | 48.5 |
| SD200) | | |
| Magnesium Stearate | Lubricant | 1.0 |
| Ligamed MF-2-V) | | |
| Colloidal Silicon | Glidant | 0.5 |
| Dioxide (Aerosil | | |
| 200P) | | |
| Total Fill Weight | | 100 |
| Capsules^{a}, Size 0 | Capsule | 1 unit |
| | Shell | |

^{a}Swedish orange Vcap capsules

The Prevotella histicola strain referred to above has been deposited as Prevotella histicola Strain B (NRRL accession number B 50329).

The Prevotella histicola (lyophilized) powder in Table 17 further included at least one spirulina component.

The capsule was banded with an HPMC-based banding solution.

The banded capsule was enteric coated with Eudragit L30-D55, a poly(methacrylic acid-co-ethyl acrylate) copolymer.

TABLE 18
Quantification of the Amount of the Components of Spirulina Detected in PharmaceuticalAgents Manufactured in Different Processes

| | Pro- | Spiru- | | Extrac- | | | Prevotella | | | | | | |
|---|---|---|---|---|---|---|---|---|---|---|---|---|---|
| Sample | cess | lina | TCC | tion | ng/ul | ng | Strain B | ChIG | ΔCt | Percentage | cpcA | ΔCt | Percentage |
| Prevotella | 1 | No | 5.3E+11 | #1 | 50 | 25.0 | 17.03 | Unde- | | | Unde- | | |
| Strain B | | | | | | | | tected | | | tected | | |
| preparations | | | | #2 | 49 | 24.5 | 17.41 | Unde- | | | Unde- | | |
| | | | | | | | | tected | | | tected | | |
| | 2 | No | 5.4E+11 | #1 | 47 | 23.5 | 17.33 | Unde- | | | Unde- | | |
| | | | | | | | | tected | | | tected | | |
| | | | | #2 | 42 | 21.0 | 17.07 | Unde- | | | Unde- | | |
| | | | | | | | | tected | | | tected | | |
| | 3 | Yes | 1.4E+12 | #1 | 79 | 39.5 | 16.15 | 29.81 | 13.67 | 0.01% | 29.02 | 12.87 | 0.01% |
| | | | | #2 | 65 | 32.5 | 16.37 | 29.56 | 13.19 | 0.01% | 29.19 | 12.83 | 0.01% |
| | 4 | Yes | 2.0E+12 | #1 | 104 | 52.0 | 15.52 | 39.59 | 24.07 | 0.000006% | 39.43 | 23.91 | 0.000006% |
| | | | | #2 | 145 | 72.5 | 14.69 | 38.65 | 23.96 | 0.000006% | 39.33 | 24.64 | 0.000004% |
| Prevotella | | Yes | | | 23 | 11.5 | 18.27 | 37.40 | 19.13 | 0.0002% | 36.76 | 18.49 | 0.0003% |
| Strain B | | | | | | | | | | | | | |
| reference | | | | | | | | | | | | | |
| batch | | | | | | | | | | | | | |
| Non- | | No | 1.2E+12 | | 192 | 96.0 | Unde- | Unde- | | | Unde- | | |
| hemoglobin | | | | | | | tected | tected | | | tected | | |
| dependent | | | | | | | | | | | | | |
| strain | | | | | | | | | | | | | |

Prevotella Strain B colum shows the results of Prevotella Strain B strain-specific qPCR
ChIG columns show the results of chlorophyll synthase spirulina-specific qPCR
cpcA columns show the results of C-phycocyanin alpha subunit spirulina-specific qPCR

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A pharmaceutical composition comprising:
a pharmaceutical agent, wherein the pharmaceutical agent comprises
(a) bacteria and/or microbial extracellular vesicles (mEVs); and
(b) at least one component of spirulina.

2. The pharmaceutical composition of claim 1, wherein the at least one component of spirulina comprises a spirulina nucleic acid.

3. The pharmaceutical composition of claim 2, wherein the spirulina nucleic acid is spirulina DNA.

4. The pharmaceutical composition of claim 3, wherein the spirulina DNA comprises a sequence encoding C-phycocyanin alpha subunit (cpcA) or chlorophyll a synthase (ChIG).

5. The pharmaceutical composition of claim 1, wherein the at least one component of spirulina comprises a spirulina protein.

6. The pharmaceutical composition of claim 5, wherein the spirulina protein is phycocyanin.

7. The pharmaceutical composition of claim 1, wherein the at least one component of spirulina comprises a spirulina small molecule.

8. The pharmaceutical composition of claim 7, wherein the spirulina small molecule is a spirulina pigment.

9. The pharmaceutical composition of claim 8, wherein the spirulina pigment is spirulina is chlorophyllin or beta carotene.

10. The pharmaceutical composition of any one of claims 1 to 9, wherein the pharmaceutical agent comprises bacteria.

11. The pharmaceutical composition of claim 10, wherein the bacteria are hemoglobin-dependent bacteria.

12. The pharmaceutical composition of claim 10 or 11, wherein the bacteria are of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptomphilus, Peptostreptococcus, Porphyromonas, Prevotella, Propionibacterium, Rarimicrobium, Shuttleworthia, Turicibacter, or Veillonella.

13. The pharmaceutical composition of claim 12, wherein the bacteria are of the genus Fournierella.

14. The pharmaceutical composition of claim 13, wherein the Fournierella are Fournierella Strain B (ATCC Deposit Number PTA-126696).

15. The pharmaceutical composition of claim 12, wherein the bacteria are of the genus Prevotella.

16. The pharmaceutical composition of claim 15, wherein the bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oxalis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

17. The pharmaceutical composition of claim 15, wherein the bacteria are of the species Prevotella histicola.

18. The pharmaceutical composition of claim 15, wherein the Prevotella comprise at least 99% genomic, 16S and/or CRISPR sequence identity to the nucleotide sequence of the Prevotella Strain B 50329 (NRRL accession number B 50329).

19. The pharmaceutical composition of claim 15, wherein the Prevotella comprise at least 99% genomic, 16S and/or CRISPR sequence identity to the nucleotide sequence of the Prevotella Strain C (ATTC Deposit Number PTA-126140).

20. The pharmaceutical composition of claim 15, wherein the Prevotella are Prevotella Strain B 50329 (NRRL accession number B 50329).

21. The pharmaceutical composition of claim 15, wherein the Prevotella are Prevotella Strain C (ATTC Deposit Number PTA-126140).

22. The pharmaceutical composition of claim 15, wherein the Prevotella bacteria (i) comprise one or more proteins listed in Table 1, and/or (ii) are substantially free of a protein listed in Table 2.

23. The pharmaceutical composition of any one of claims 10 to 22, wherein the bacterial are live, attenuated, or dead.

24. The pharmaceutical composition of any one of claims 10 to 23, wherein the bacteria are lyophilized bacteria.

25. The pharmaceutical composition of any one of claims 1-24, wherein the pharmaceutical agent comprises mEVs.

26. The pharmaceutical composition of claim 25, wherein the mEVs are secreted mEVs (smEVs).

27. The pharmaceutical composition of claim 25, wherein the mEVs are processed mEVs (pmEVs).

28. The pharmaceutical of any one of claims 25 to 27, wherein the mEVs are from hemoglobin-dependent bacteria.

29. The pharmaceutical composition of any one of claims 25 to 28, wherein the mEVs are from bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptomphilus, Peptostreptococcus, Porphyromonas, Prevotella, Propionibacterium, Rarimicrobium, Shuttleworthia, Turicibacter, or Veillonella.

30. The pharmaceutical composition of claim 29, wherein the mEVs are from bacteria of the genus Fournierella.

31. The pharmaceutical composition of claim 30, wherein the Fournierella are Fournierella Strain B (ATCC Deposit Number PTA-126696).

32. The pharmaceutical composition of claim 29, wherein the mEVs are from bacteria of the genus Prevotella.

33. The pharmaceutical composition of claim 32, wherein the bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oxalis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

34. The pharmaceutical composition of claim 32, wherein the mEVs are from bacteria of the species Prevotella histicola.

35. The pharmaceutical composition of claim 32, wherein the Prevotella comprise at least 99% genomic, 16S and/or CRISPR sequence identity to the nucleotide sequence of the Prevotella Strain B 50329 (NRRL accession number B 50329).

36. The pharmaceutical composition of claim 32, wherein the Prevotella comprise at least 99% genomic, 16S and/or CRISPR sequence identity to the nucleotide sequence of the Prevotella Strain C (ATTC Deposit Number PTA-126140).

37. The pharmaceutical composition of claim 32, wherein the Prevotella are Prevotella Strain B 50329 (NRRL accession number B 50329).

38. The pharmaceutical composition of claim 32, wherein the Prevotella are Prevotella Strain C (ATTC Deposit Number PTA-126140).

39. The pharmaceutical composition of claim 32, wherein the Prevotella bacteria (i) comprise one or more proteins listed in Table 1, and/or (ii) are substantially free of a protein listed in Table 2.

40. The pharmaceutical composition of any one of claims 25 to 39, wherein the mEVs are lyophilized mEVs.

41. The pharmaceutical composition of any one of claims 1 to 40, further comprising a cryoprotectant.

42. A solid dosage form comprising:
(i) a pharmaceutical agent, wherein the pharmaceutical agent comprises
(a) bacteria and/or microbial extracellular vesicles (mEVs); and
(b) at least one component of spirulina; and
(ii) at least one diluent, at least one lubricant, at least one glidant, and/or at least one disintegration agent.

43. The solid dosage form of claim 42, wherein the at least one diluent has a total mass that is at least 1% and no more than 95% of the total mass of the solid dosage form.

44. The solid dosage form of claim 42, wherein the at least one diluent comprises mannitol.

45. The solid dosage form of any one of claims 42 to 44, wherein the at least one lubricant has a total mass that is at least 0.1% and no more than 5% of the total mass of the solid dosage form.

46. The solid dosage form of any one of claims 42 to 45, wherein the at least one lubricant comprises magnesium stearate.

47. The solid dosage form of any one of claims 42 to 46, wherein the at least one glidant has a total mass that is at least 0.01% and no more than 2% of the total mass of the solid dosage form.

48. The solid dosage form of any one of claims 42 to 47, wherein the at least one glidant comprises colloidal silicon dioxide.

49. The solid dosage form of any one of claims 42 to 48, wherein the at least one disintegration agent has a total mass that is at least 40% of the total mass of the solid dosage form.

50. The solid dosage form of any one of claims 42 to 49, wherein the at least one disintegration agent comprises low-substituted hydroxypropyl cellulose (L-HPC), croscarmellose sodium (Ac-Di-Sol), and/or crospovidone (PVPP).

51. The solid dosage form of any one of claims 42 to 50, wherein the the at least one disintegration agent comprises low-substituted hydroxypropyl cellulose (L-HPC), croscarmellose sodium (Ac-Di-Sol), and crospovidone (PVPP).

52. The solid dosage form of claim 50 or 51, wherein the L-HPC has a total L-HPC mass that is at least 22% and no more than 42% of the total mass of the solid dosage form.

53. The solid dosage form of any one of claims 50 to 52, wherein the L-HPC is L-HPC of grade LH-B1.

54. The solid dosage form of any one of claims 50 to 53, wherein the Ac-Di-Sol has a total Ac-Di-Sol mass that is at least 0.01% and no more than 16% of the total mass of the solid dosage form.

55. The solid dosage form of any one of claims 50 to 54, wherein the Ac-Di-Sol is Ac-Di-Sol of grade SD-711.

56. The solid dosage form of any one of claims 50 to 55, wherein the PVPP has a total PVPP mass that is at least 5% and no more than 25% of the total mass of the solid dosage form.

57. The solid dosage form of any one of claims 50 to 56, wherein the total L-HPC mass plus the total Ac-Di-Sol mass plus the total PVPP mass is at least 40% of the total mass of the solid dosage form.

58. The solid dosage form of any one of claims 50 to 57, wherein
the total L-HPC mass is at least 22% and no more than 42% of the total mass of the solid dosage form;
the total Ac-Di-Sol mass is at least 0.01% and no more than 16% of the total mass of the solid dosage form; and
the total PVPP mass is at least 5% and no more than 25% of the total mass of the solid dosage form.

59. The solid dosage form of any one of claims 50 to 58, wherein
the total L-HPC mass is about 32% of the total mass of the solid dosage form;
the total Ac-Di-Sol mass is about 6% of the total mass of the solid dosage form; and
the total PVPP mass is about 15% of the total mass of the solid dosage form.

60. The solid dosage form of any one of claims 42 to 59, wherein the pharmaceutical agent has a total pharmaceutical agent mass that is at least 5% and no more than 65% of the total mass of the solid dosage form.

61. The solid dosage form of claim 60, wherein the pharmaceutical agent has a total pharmaceutical agent mass that is at least 5% and no more than 35% of the total mass of the solid dosage form.

62. The solid dosage form of claim 60, wherein the total pharmaceutical agent mass is about 25% of the total mass of the solid dosage form.

63. The solid dosage form of any one of claims 42 to 62, wherein the solid dosage form is a tablet.

64. The solid dosage form of claim 63, wherein tablet is a 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, or 18 mm tablet.

65. The solid dosage form of any one of claims 42 to 62, wherein the solid dosage form is a minitablet.

66. The solid dosage form of claim 65, wherein the minitablet is a 1 mm minitablet, 1.5 mm minitablet, 2 mm minitablet, 3 mm minitablet, or 4 mm minitablet.

67. The solid dosage form of claim 65 or 66, wherein a plurality of minitablets are contained in a capsule.

68. The solid dosage form of any one of claims 42 to 67, further comprising an enteric coating.

69. The solid dosage form of claim 68, wherein the enteric coating is a single enteric coating or more than one enteric coating.

70. The solid dosage form of claim 68 or 69, wherein the enteric coating comprises an inner enteric coating and an outer enteric coating, and wherein the inner and outer enteric coatings are not identical.

71. The solid dosage form of claim any one of claims 68 to 70, wherein the enteric coating comprises a methacrylic acid ethyl acrylate (MAE) copolymer (1:1).

72. The solid dosage form of any one of claims 68 to 71, wherein the enteric coating comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate.

73. The solid dosage form of any one of claims 68 to 72, wherein the enteric coating comprises an anionic polymeric material.

74. A solid dosage form comprising:
(i) a pharmaceutical agent, wherein the pharmaceutical agent comprises
(a) bacteria and/or microbial extracellular vesicles (mEVs); and
(b) at least one component of spirulina; and
(ii) at least one diluent, at least one lubricant, and/or at least one glidant.

75. The solid dosage form of claim 74, wherein the at least one diluent has a total mass that is at least 1% and no more than 95% of the total mass of the solid dosage form.

76. The solid dosage form of claim 74 or 75, wherein the at least one diluent comprises mannitol.

77. The solid dosage form of any one of claims 74 to 76, wherein the at least one lubricant has a total mass that is at least 0.1% and no more than 5% of the total mass of the solid dosage form.

78. The solid dosage form of any one of claims 74 to 77, wherein the at least one lubricant comprises magnesium stearate.

79. The solid dosage form of any one of claims 74 to 78, wherein the at least one glidant has a total mass that is at least 0.01% and no more than 2% of the total mass of the solid dosage form.

80. The solid dosage form of any one of claims 74 to 79, wherein the at least one glidant comprises colloidal silicon dioxide.

81. The solid dosage form of any one of claims 74 to 80, wherein the pharmaceutical agent has a total pharmaceutical agent mass that is at least 5% and no more than 95% of the total mass of the solid dosage form.

82. The solid dosage form of claim 81, wherein the pharmaceutical agent has a total pharmaceutical agent mass that is at least 20% and no more than 50% of the total mass of the solid dosage form.

83. The solid dosage form of claim 81, wherein the total pharmaceutical agent mass is about 30% to about 50% of the total mass of the solid dosage form.

84. The solid dosage form of any one of claims 74 to 83, wherein
(i) the total pharmaceutical agent mass is at least 5% and no more than 95% of the total mass of the solid dosage form;
(ii) the total diluent mass is at least 1% and no more than 95% of the total mass of the solid dosage form;
(iii) the total lubricant mass is at least 0.1% and no more than 5% of the total mass of the solid dosage form; and
(iv) the total glidant mass is at least 0.01% and no more than 2% of the total mass of the solid dosage form.

85. The solid dosage form of any one of claims 74 to 83, wherein
(i) the total pharmaceutical agent mass is about 20% to about 50% of the total mass of the solid dosage form;
(ii) the total diluent mass is about 50% to 80% of the total mass of the solid dosage form; (iii) the total lubricant mass is about 1% of the total mass of the solid dosage form; and (iv) the total glidant mass is about 0.5% of the total mass of the solid dosage form.

86. The solid dosage form of any one of claims 74 to 83, wherein
(i) the total pharmaceutical agent mass is about 30% to about 50% of the total mass of the solid dosage form;
(ii) the total diluent mass is about 45% to 70% of the total mass of the solid dosage form; (iii) the total lubricant mass is about 1% of the total mass of the solid dosage form; and (iv) the total glidant mass is about 0.5% of the total mass of the solid dosage form.

87. The solid dosage form of any one of claims 74 to 83, wherein
(i) the total pharmaceutical agent mass is about 50% of the total mass of the solid dosage form;
(ii) the total diluent mass is about 48.5% of the total mass of the solid dosage form;
(iii) the total lubricant mass is about 1% of the total mass of the solid dosage form; and (iv) the total glidant mass is about 0.5% of the total mass of the solid dosage form.

88. The solid dosage form of any one of claims 74 to 83, wherein
(i) the total pharmaceutical agent mass is about 13.51% of the total mass of the solid dosage form;
(ii) the total diluent mass is about 84.99% of the total mass of the solid dosage form;
(iii) the total lubricant mass is about 1% of the total mass of the solid dosage form; and (iv) the total glidant mass is about 0.5% of the total mass of the solid dosage form.

89. The solid dosage form of any one of claims 74 to 83, wherein
(i) the total pharmaceutical agent mass is about 90.22% of the total mass of the solid dosage form;
(ii) the total diluent mass is about 8.28% of the total mass of the solid dosage form;
(iii) the total lubricant mass is about 1% of the total mass of the solid dosage form;
and (iv) the total glidant mass is about 0.5% of the total mass of the solid dosage form.

90. The solid dosage form of any one of claims 74 to 89, wherein the solid dosage form is a capsule.

91. The solid dosage form of claim 90, wherein the capsule is a size 00, size 0, size 1, size 2, size 3, size 4, or size 5 capsule.

92. The solid dosage form of claim 90 or 91, wherein the capsule is a size 0 capsule.

93. The solid dosage form of any one of claims 74 to 92, further comprising an enteric coating.

94. The solid dosage form of claim 93, wherein the solid dosage form is enteric coated to dissolve at pH 5.5.

95. The solid dosage form of claim 93 or 94, wherein the enteric coating comprises a polymethacrylate-based copolymer.

96. The solid dosage form of any one of claims 93 to 95, wherein the the enteric coating comprises poly(methacrylic acid-co-ethyl acrylate).

97. The solid dosage form of any one of claims 93 to 96, wherein the enteric coating comprises a methacrylic acid ethyl acrylate (MAE) copolymer (1:1) (e.g., Kollicoat MAE 100P).

98. The solid dosage form of any one of claims 93 to 97, wherein the enteric coating comprises a Eudragit copolymer, e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

99. The solid dosage form of any one of claims 93 to 98, wherein the enteric coating comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate.

100. The solid dosage form of any one of claims 93 to 99, wherein the enteric coating comprises an anionic polymeric material.

101. A method of preventing or treating a disease of a subject comprising administering to the subject a pharmaceutical composition of any one of claims 1 to 41.

102. A method of preventing or treating a disease in a subject comprising administering to the subject a solid dosage form of any one of claims 42 to 100.

103. Use of a pharmaceutical composition of any one of claims 1 to 41 for the treatment or prevention of a disease of a subject.

104. Use of a solid dosage form of any one of claims 42 to 100 for the treatment or prevention of a disease in a subject.

105. The method or use of any one of claims 101 to 104, wherein the disease is a cancer, inflammation, autoimmunity, a metabolic condition, or a dysbiosis.

106. The method or use of any one of claims 101 to 104, wherein the disease is bacterial septic shock, cytokine storm and/or viral infection (such as a coronavirus infection, an influenza infection, and/or a respiratory syncytial virus infection).

107. The method or use of any one of claims 101 to 104, wherein the solid dosage form decreases inflammatory cytokine expression (e.g., decreased IL-8, IL-6, IL-1β, and/or TNFα expression levels).

108. A method of preparing a solid dosage form, the method comprising:
(A) combining into a pharmaceutical composition:
(i) a pharmaceutical agent, wherein the pharmaceutical agent comprises
(a) bacteria and/or microbial extracellular vesicles (mEVs); and
(b) at least one component of spirulina; and
(ii) at least one diluent, at least one lubricant, at least one glidant, and/or at least one disintegration agent.
(B) compressing the pharmaceutical composition into a solid dosage form.

109. The method of claim 108, further comprising the step of enterically coating the solid dosage form to obtain an enterically coated solid dosage form.

110. The method of claim 108 or 109 wherein the solid dosage form is a tablet.

111. The method of claim 108 or 109, wherein the solid dosage form is a minitablet.

112. A method of preparing a solid dosage form, the method comprising combining into a pharmaceutical composition:
(i) a pharmaceutical agent, wherein the pharmaceutical agent comprises
(a) bacteria and/or microbial extracellular vesicles (mEVs); and
(b) at least one component of spirulina; and
(ii) at least one diluent, at least one lubricant, and/or at least one glidant.

113. The method of claim 112, further comprising blending and/or loading the pharmaceutical composition into a capsule.

114. The method of claim 113, further comprising banding the capsule.

115. The method of claim 114, wherein the capsule is banded with an HPMC-based banding solution.

116. The method of any one of claims 112 to 115, further comprising the step of enterically coating the solid dosage form to obtain an enterically coated solid dosage form.

117. The method of any one of claims 112 to 116, wherein the solid dosage form is a capsule.

118. A method of testing a pharmaceutical composition comprising bacteria and/or microbial extracellular vesicles (mEVs), the method comprising performing an assay to detect the presence of a component of spirulina in the pharmaceutical composition.

119. The method of claim 118, wherein the component of spirulina comprises a spirulina nucleic acid.

120. The method of claim 119, wherein the spirulina nucleic acid is spirulina DNA

121. The method of claim 120, wherein the spirulina DNA comprises a sequence encoding C-phycocyanin alpha subunit (cpcA) or chlorophyll a synthase (ChIG).

122. The method of claim any one of claims 119 to 121, wherein the assay to detect the presence of a component of spirulina is a nucleic acid amplification assay, a sequencing assay, and/or a microarray assay.

123. The method of any one of claims 119 to 121, the assay to detect the presence of a component of spirulina is a polymerase chain reaction (PCR) assay.

124. The method of claim 118, wherein the component of spirulina is a spirulina protein.

125. The method of claim 124, wherein the spirulina protein is phycocyanin.

126. The method of claim 124 or 125, wherein the spirulina protein is detected using an antibody specific for the spirulina protein, HPLC or UPLC.

127. The method of claim 118, wherein the component of spirulina comprises a spirulina small molecule.

128. The method of claim 127, wherein the spirulina small molecule is a spirulina pigment.

129. The method of claim 128, wherein the spirulina pigment is spirulina is chlorophyllin or beta carotene.

130. The method of claim 128 or 129, wherein the spirulina pigment is detected by HPLC or UPLC.

131. The method of any one of claims 118 to 130, wherein the pharmaceutical composition comprises bacteria.

132. The method of claim 131, wherein the bacteria are hemoglobin-dependent bacteria.

133. The method of claim 131 or 132, wherein the bacteria are of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, Turicibacter, or Veillonella.

134. The method of claim 133, wherein the bacteria are of the genus Fournierella.

135. The method of claim 134, wherein the Fournierella are Fournierella Strain B (ATCC Deposit Number PTA-126696).

136. The method of claim 133, wherein the bacteria are of the genus Prevotella.

137. The method of claim 136, wherein the bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oxalis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

138. The method of claim 136, wherein the bacteria are of the species Prevotella histicola.

139. The method of claim 136, wherein the Prevotella comprise at least 99% genomic, 16S and/or CRISPR sequence identity to the nucleotide sequence of the Prevotella Strain B 50329 (NRRL accession number B 50329).

140. The method of claim 136, wherein the Prevotella comprise at least 99% genomic, 16S and/or CRISPR sequence identity to the nucleotide sequence of the Prevotella Strain C (ATTC Deposit Number PTA-126140).

141. The method of claim 136, wherein the Prevotella are Prevotella Strain B 50329 (NRRL accession number B 50329).

142. The method of claim 136, wherein the Prevotella are Prevotella Strain C (ATTC Deposit Number PTA-126140).

143. The method of claim 136, wherein the Prevotella bacteria (i) comprise one or more proteins listed in Table 1, and/or (ii) are substantially free of a protein listed in Table 2.

144. The method of any one of claims 131 to 143, wherein the bacterial are live, attenuated, or dead.

145. The method of any one of claims 131 to 144, wherein the bacteria are lyophilized bacteria.

146. The method of any one of claims 118 to 145, wherein the pharmaceutical composition comprises mEVs.

147. The method of claim 146, wherein the mEVs are secreted mEVs (smEVs).

148. The method of claim 146, wherein the mEVs are processed mEVs (pmEVs).

149. The method of any one of claims 146 to 148, wherein the mEVs are from hemoglobin-dependent bacteria.

150. The method of any one of claims 146 to 149, wherein the mEVs are from bacteria of the genus Actinomyces, Alistipes, Anaerobutyricum, Bacillus, Bacteroides, Cloacibacillus, Clostridium, Collinsella, Cutibacterium, Eisenbergiella, Erysipelotrichaceae, Eubacterium/Mogibacterium, Faecalibacterium, Fournierella, Fusobacterium, Megasphaera, Parabacteroides, Peptoniphilus, Peptostreptococcus, Porphyromonas, Prevotella, Propionibacterium, Rarimicrobium, Shuttleworthia, Turicibacter, or Veillonella.

151. The method of claim 150, wherein the mEVs are from bacteria of the genus Fournierella.

152. The method of claim 151, wherein the Fournierella are Fournierella Strain B (ATCC Deposit Number PTA-126696).

153. The method of claim 150, wherein the mEVs are from bacteria of the genus Prevotella.

154. The method of claim 153, wherein the bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oxalis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

155. The method of claim 153, wherein the mEVs are from bacteria of the species Prevotella histicola.

156. The method of claim 153, wherein the Prevotella comprise at least 99% genomic, 16S and/or CRISPR sequence identity to the nucleotide sequence of the Prevotella Strain B 50329 (NRRL accession number B 50329).

157. The method of claim 153, wherein the Prevotella comprise at least 99% genomic, 16S and/or CRISPR sequence identity to the nucleotide sequence of the Prevotella Strain C (ATTC Deposit Number PTA-126140).

158. The method of claim 153, wherein the Prevotella are Prevotella Strain B 50329 (NRRL accession number B 50329).

159. The method of claim 153, wherein the Prevotella are Prevotella Strain C (ATTC Deposit Number PTA-126140).

160. The method of claim 153, wherein the Prevotella bacteria (i) comprise one or more proteins listed in Table 1, and/or (ii) are substantially free of a protein listed in Table 2.
